SEQUENCE LISTING (225 sequences)

SEQ ID NO: 1 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 2 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 3 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 4 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 5 - DNA, 969 bp, Streptococcus thermophilus
  source 1..969: mol_type = genomic DNA
atgagtaagaaactcttaggtattgaccttggtggaacaactgttaagtttggtattttgaatgcagatggtgaagttcaagaaaaatgggctattggaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgtcatcgttgatggtaaattaattcatggtgttgccggtgctggtggggaaattggtcacattattgttgaacctgacacaggatttgagtgtacttgcggaaacaaggggtgtctggaaactgtagcttcagcaacaggtattgtacgtatagcacatcatttggcagaaaaatacgaaggaaactcttctattaaagctgctgtagacaatggtgagtttgtgacaagtaaagatattctcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagccgctagtcttgcttatagtattgacaaataa

SEQ ID NO: 6 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 7 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagcttttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtagctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 8 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 9 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgcagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 10 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 11 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtagctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaatgggtgtctggaaactgtagcttcagcaacaggtattgtacgtgtaacacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 12 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 13 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagactctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 14 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 15 - DNA, 969 bp, Streptococcus thermophilus
  source 1..969: mol_type = genomic DNA
atgagtaagaaactcttaggtattgaccttggtggaacaactgttaagtttggtattttgaatgcagatggtgaagttcaagaaaaatgggctattggaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttgtaacattgggtacaggtgttggtggcggtgtcatcgttgatggtaaattaattcatggtgttgccggtgctggtggggaaattggtcacattattgttgaacctgacacaggatttgagtgtacttgcggaaacaaggggtgtctggaaactgtagcttcagcaacaggtattgtacgtatagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 16 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 17 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgataatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggttctgttattgaaaaagaacttggtattccattcgctattgataatgatgctaatgtggctgcactgggtgaacgttgggttggtgctggtgctaacagtcgggatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgttgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 18 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 19 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgataatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctattattgaaaaagaacttggtattccattcgctattgataatgatgctaatgtggctgcactgggtgaacgttgggttggtgctggtgctaacaatcgggatgttgtctttgtaacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagcttgtgacaagtaaagatattctcgtagctgctactgaaggtgataagtttgctgacagcattgttgataaagtctctaaatacctcggacttgcaatagcaaacatctcaaacattcttaacccagattctgtcattatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 20 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 21 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttggtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggaaaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 22 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 23 - protein, 322 aa, Streptococcus thermophilus
MSKKLLGIDLGGTTVKFGILTADGEVQEKWAIETNTFENGSHIVPDIVESLKHRLELYGLTAEDFIGIGMGPPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 24 - DNA, 147 bp, Streptococcus thermophilus
  source 1..147: mol_type = genomic DNA
catagtatcctcctcatatttcagtataacataacttttattttttacctatattttactaaaaaaatagtaaaaatattgattttccatgtgaaaggggttatgatttcagtataaacaaaaagaataagtgagatacatcctatg

SEQ ID NO: 25 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 26 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 27 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 28 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 29 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 30 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 31 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGXFLRSRVEGYFTRYAFPQVRRTTKVKLAELGNDAGIIGAASLAYSIDK

SEQ ID NO: 32 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGXFLRSRVEGYFTRYAFPQVRRTTKVKLAELGNDAGIIGAASLAYSIDK

SEQ ID NO: 33 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGXFLRSRVEGYFTRYAFPQVRRTTKVKLAELGNDAGIIGAASLAYSIDK

SEQ ID NO: 34 - protein, 322 aa, Streptococcus thermophilus
  VAR_SEQ 275: note = MISC_FEATURE - Xaa can be any naturally occurring amino acid, but not Glu
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGXFLRSRVEGYFTRYAFPQVRRTTKVKLAELGNDAGIIGAASLAYSIDK

SEQ ID NO: 35 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 36 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 37 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 38 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 39 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVGGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 40 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGKFLRSRVEGYFTRYAFPQVRRTTKVKLAELGNDAGIIGAASLAYSIDK

SEQ ID NO: 41 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVGGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGKFLRSRVEGYFTRYAFPQVRRTTKVKLAELGNDAGIIGAASLAYSIDK

SEQ ID NO: 42 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGKFLRSRVEGYFTRYAFPQVRRTTKVKLAELGNDAGIIGAASLAYSIDK

SEQ ID NO: 43 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVGGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGKFLRSRVEGYFTRYAFPQVRRTTKVKLAELGNDAGIIGAASLAYSIDK

SEQ ID NO: 44 - DNA, 969 bp, Streptococcus thermophilus
  source 1..969: mol_type = genomic DNA
atgagtaagaaactcttaggtattgaccttggtggaacaactgttaagtttggtattttgactgcagatggtgaagttcaagaaaaatgggctattgaaacaaatacgtttgaaaatggtagccacattgttcctgacattgtagaatctttgaaacaccgtttggaattgtatggacttactgctgaagattttattggaattggtatgggatctccaggtgcagttgaccgagaaaataaaacagtaacgggtgcctttaacttgaactgggcagaaactcaagaagttggctctgttattgaaaaagaacttggtattccattcgctattgataatgatgctaatgtggctgcactgggtgaacgttgggttggtgctggtgctaacaatcggaatgttgtctttataacattgggtacaggtgttagtggcggtgttatcgctgatggtaacttaattcatggtgttgccggtgctggtggggaaattggtcacattattgttgaacctgacacaggatttgagtgtacttgcggaaacaaggggtgtctggaaactgtagcttcagcaacaggtattgtacgtgtagcacatcatttggcagaaaaatacgaaggaaactcttctattaaagctgctgtagacaatggtgagtttgtgacaagtaaagatattatcgtagctgctactgaaggtgataagtttgctgacagcattgttgataaagtctctaaatacctcggacttgcaacagcaaacatctcaaacattcttaacccagattctgtcgttatcggtggtggtgtttctgccgcaggagaattcttgcgtagtcgtgttgaaggatactttacacgttatgcattcccacaagttcgccgtacaacaaaagtgaaattagcggagcttggaaatgatgcaggaatcattggagctgctagtcttgcttatagtattgacaaataa

SEQ ID NO: 45 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 46 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 47 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 48 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 49 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 50 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 51 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVXGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 52 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 53 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVXGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 54 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 55 - protein, 322 aa, Streptococcus thermophilus
  VAR_SEQ 144: note = MISC_FEATURE - Xaa can be any naturally occurring amino acid, but not Gly
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVXGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 56 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 57 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIVDGKLIHGVAGAGGEIGHIIVEPDTGFECTCGNKGCLETVASATGIVRIAHHLAEKYEGNSSIKAAVDNGEFVTSKDILVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 58 - protein, 322 aa, Streptococcus thermophilus
MSKKLLGIDLGGTTVKFGILTADGEVQEKWAIETNTFENGSHIVPDIVESLKHRLELYGLTAEAFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETVASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 59 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNKGCLETAASATGIVRVAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 60 - protein, 322 aa, Streptococcus thermophilus
MSKKLLGIDLGGTTVKFGILTADGEVQEKWAIETNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFITLGTGVSGGVIADGNLIHGVAGAGGEIGHIIVEPDTGFECTCGNNGCLETVASATGIVRVTHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 61 - protein, 322 aa, Streptococcus thermophilus
MSKKLLGIDLGGTTVKFGILTADGEVQEKWAIETNTFENGSHIVPDIVDSLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 62 - protein, 322 aa, Streptococcus thermophilus
MSKKLLGIDLGGTTVKFGILNADGEVQEKWAIGTNTFENGSHIVPDIVESLKHRLELYGLTAEDFIGIGMGSPGAVDRENKTVTGAFNLNWAETQEVGSVIEKELGIPFAIDNDANVAALGERWVGAGANNRNVVFVTLGTGVSGGVIVDGKLIHGVAGAGGEIGHIIVEPDTGFECTCGNKGCLETVASATGIVRIAHHLAEKYEGNSSIKAAVDNGEFVTSKDIIVAATEGDKFADSIVDKVSKYLGLATANISNILNPDSVVIGGGVSAAGEFLRSRVEGYFTRYAFPQVRRTTKVKLAELGNDAGIIGAASLAYSIDK

SEQ ID NO: 63 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSVIEKELGIPFAIDNDANVAALGERWVGAGANSRDVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVVIGGGVSAAGEFLRSRVEGYFTRYAFPQVRRTTKVKLAELGNDAGIIGAASLAYSIDK

SEQ ID NO: 64 - protein, 322 aa, Streptococcus thermophilus
MSKKLLGIDLGGTTVKFGILTADGEVQEKWAIETNTFDNGSHIVPDIVESLKHRLELYGLTAEDFIGIGMGSPGAVDRENKTVTGAFNLNWAETQEVGSIIEKELGIPFAIDNDANVAALGERWVGAGANNRDVVFVTLGTGVSGGVIADGNLIHGVAGAGGEIGHIIVEPDTGFECTCGNKGCLETVASATGIVRVAHHLAEKYEGNSSIKAAVDNGELVTSKDILVAATEGDKFADSIVDKVSKYLGLAIANISNILNPDSVIIGGGVSAAGEFLRSRVEGYFTRYAFPQVRRTTKVKLAELGNDAGIIGAASLAYSIDK

SEQ ID NO: 65 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggccgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 66 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgatttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 67 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccaaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 68 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 69 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 70 - DNA, 1002 bp, Streptococcus thermophilus
  source 1..1002: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaatgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 71 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggccgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 72 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgatttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 73 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccaaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 74 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 75 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaaataccaagtgttaacatcgactataaagctgccgttgaggattgtgtaacgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagataaagacgttattcttaatcatggtctaactttacgccagtcaacaaaataa

SEQ ID NO: 76 - DNA, 1001 bp, Streptococcus thermophilus
  source 1..1001: mol_type = genomic DNA
atgaatactgatgaaacaatcacaatttatgatgtagcgcgtgaagctggagtatcgatggcaactgtttctcgtgttgtaaatggtaacaaaaacgtaaaagaaaacacccgaaaaaagtgctcgaagtcattgatcgtttggattaccgtccaaatgcggttgcgcgtggcttggcaagtaaaaaaacaactactgtaggagttgtcattccaaatattgtaaatagctattttgctactctagctaaaggtattgatgacattgcaaccatgtataagtataatattgttcttgcttccagtgatgataatgaggatcatgaagttacagtcattcattctctaatttctaaacaagttgatggtattatttttatgggacaccatctgacagaaaaaatccgtgcagaattctctcgtacccgtacaccgattgttctagcaggaacagttgatctcgaacaccaattaccaagtgttaacatcgactataaagctgccgttgaggattgtgtaatgcaacttgctaaaaataatgaaaaggttgcctttgtatcaggaccactaattgatgatattaatggcaaactacgtttggctgggtataagtctggacttgaaaagaataatttgagctacaacgaaggacttgtctttgaagctaaatatagctataaagacggctttgagttagcacaacgtgtcttgaactctggtgccactgctgcctatgttggggaagatgaattggctgcaggtctcttgaatggcctctttgctgcaggcaaatcagttccagaagatttcgaaatcatcacaagcaatgattcaccggttacaagctacacacgtccaaacctttctagtataaaccatcctctctatgatttaggggcagttagcatgcgtatgttgactaaaattatgcataaggaagaacttgaagagaaagacgttattcttaatcatggtctaactttacgccagtcaacaaaataa

SEQ ID NO: 77 - DNA, 993 bp, Streptococcus thermophilus
  source 1..993: mol_type = genomic DNA
atgggtatcggtattattattgccagccatggtaggttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagatttgtttgagcttatcaagaaagctaacgttcaataa

SEQ ID NO: 78 - protein, 330 aa, Streptococcus thermophilus
MGIGIIIASHGRFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 79 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 80 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 81 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagatttgtttgagcttatcaagaaagctaacgttcaataa

SEQ ID NO: 82 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 83 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 84 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 85 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactatagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtgttattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 86 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRVIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 87 - DNA, 993 bp, Streptococcus thermophilus
  source 1..993: mol_type = genomic DNA
atgggtatcggtattattatcgccagccatggtaagttcgctgaaggaatccaccaatcaggctctatgatttttggggaccaagagaaagttcgagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 88 - protein, 330 aa, Streptococcus thermophilus
MGIGIIIASHGKFAEGIHQSGSMIFGDQEKVRVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 89 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 90 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 91 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 92 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 93 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactatagagcaagttgctgctaatatcatcaaggaagctaagggtggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 94 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 95 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgttgctaatatcatcaaggaagctaagggtggtatcaaggcacttccagaggcgctaaatccagctgaggaaacaactgcagctcctgtagaagctgcagcacttcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 96 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVVANIIKEAKGGIKALPEALNPAEETTAAPVEAAALQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 97 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattagttgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 98 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLVDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 99 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgtttttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 100 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVFPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 101 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaataacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaaattgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggactcacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgtcgaatttgacgtccgtaaggttccaaacgattctaagaaagacttgtttgagcttatcaagaaagctaacgttcaataa

SEQ ID NO: 102 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQIAANIIKEAKGGIKALPEELNPAEETTAAPVEAAAPQGAIPEGTVIGDGKLKINLARLDSRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 103 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttactgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 104 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVTANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 105 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaaagccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaggagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcgcttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 106 - protein, 330 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 107 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttgccaatgctaatccaagcatacactgaacgtatgatggatgctaacgctactgtagagcaagttgctgctaatatcatcaaggaagctaaggctggtatcaaggcacttccagaggcgctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcacttgtcttgttcgaaactgttcaagacgtacttcgtgctgtcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 108 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGVEFDVRKVPNDSKKDLFELIKKANVQ

SEQ ID NO: 109 - DNA, 993 bp, Streptococcus thermophilus
  source 1..993: mol_type = genomic DNA
atgggtatcggtattattattgccagccatggtaagttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaacccagatcgcaagattgctatcatcacaggacttaacttaccaatgctaatccaagcatacactgaacgtatgatggatgctaacgctactgtagagcaagttgctgctaatatcatcaaggaagctaaggcaggtatcaaggcacttccagaggcgctaaatccagctgaggaaacaactgcagctcctgtagaagctacagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgttgaatttgacgtccgtaaggttccaaacgattctaagaaagacttgtttgagcttatcaagaaagctaacgttcaataa

SEQ ID NO: 110 - protein, 330 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEATAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGVEFDVRKVPNDSKKDLFELIKKANVQ

SEQ ID NO: 111 - DNA, 993 bp, Streptococcus thermophilus
  source 1..993: mol_type = genomic DNA
atgggtatcggtattattattgccagccatggtaggttcgctgaaggaatccaccaatcaggctctatgatttttggggaccaagagaaagttcaagttgtgactttcatgccaagtgaaggtcctgatgatttgtacgctcacttcaacaacgccattgcacaattcgatgttgatgatgaaattcttgttttggctgacctttggagtggttcaccatttaaccaagctagtcgaatcgctagggaaaatccagatcgcaagattgctatcatcacaggacttaacttgccaatgctaatccaagcatacactgaacgtatgatggatgctaacgctactgtagagcaagttgctgctaatatcatcaaggaagctaagggtggtatcaaggcacttccagaagagctaaatccagctgaggaaacaactgcagctcctgtagaagctgcagcacctcaaggagctatccctgaaggaacagtcatcggagatggtaaactcaagattaacttggcacgtttggacacacgtctcttgcatggtcaagtagtaactaactgggtaccttattctaaagcagaccgtattattgttgcttcggatgacgttgccaaagatgagcttcgtaaggaattgatcaaacaggctgcaccaaacggtattaaagtaaacgttgttccgattcaaaaattaattgatgcttctaaagacccacgttttggaaatacacatgcgcttgtcttgttcgaaactgttcaagacgcacttcgtgctatcgaaggtggcgtgccaataaaagaacttaacgttggttctatggctcactcaactggtaaaacaatggttaacaacgttttgtctatggataaagatgatgttgcttgttttgaaaaattacgtgaccttggcgtttaatttgacgtccgtaaggttccaaacgattctaagaaagatttgtttgagcttatcaagaaagctaacgttcaataa

SEQ ID NO: 112 - protein, 305 aa, Streptococcus thermophilus
MGIGIIIASHGRFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 113 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 114 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 115 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGV

SEQ ID NO: 116 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRVIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 117 - protein, 305 aa, Streptococcus thermophilus
MGIGIIIASHGKFAEGIHQSGSMIFGDQEKVRVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 118 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 119 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 120 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATIEQVAANIIKEAKGGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 121 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVVANIIKEAKGGIKALPEALNPAEETTAAPVEAAALQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 122 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLVDASKDPRFGNTHALVLFETVQDVLRAVEGGVPIKELNVGSMAHSTGKTMVNNVLSMDKDDVACFEKLRDLGV

SEQ ID NO: 123 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVFPIQKLIDASKDPRFGNTHALVLFETVQDALRAIEGGVPIKELNVGSMAHSTGKTMVNNVLSMDKDDVACFEKLRDLGV

SEQ ID NO: 124 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQIAANIIKEAKGGIKALPEELNPAEETTAAPVEAAAPQGAIPEGTVIGDGKLKINLARLDSRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 125 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVTANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 126 - protein, 305 aa, Streptococcus thermophilus
MGIGIIIASHGKFAEGIHQSGSMIFGDQEKVQVVTFMPSEGPDDLYAHFNKAIAQFDVDDEILVLADLWSGSPFNQASRIARENPDRKIAIITGLNLPMLIQAYTERMMDANATVEQVAANIIKEAKGGIKALPEELNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 127 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEAAAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDVLRAVEGGVPIKELNVGSMAHSTGKTMVNNVLSMDKDDVACFEKLRDLGV

SEQ ID NO: 128 - protein, 305 aa, Streptococcus thermophilus
MGIGIIIASHGKFAEGIHQSGSMIFGDQEKVQVVTFMPSEGPDDLYAHFNNAIAQFDVDDEILVLADLWSGSPFNQASRIARENPDRKIAIITGLNLPMLIQAYTERMMDANATVEQVAANIIKEAKAGIKALPEALNPAEETTAAPVEATAPQGAIPEGTVIGDGKLKINLARLDTRLLHGQVVTNWVPYSKADRIIVASDDVAKDELRKELIKQAAPNGIKVNVVPIQKLIDASKDPRFGNTHALVLFETVQDALRAIEGGVPIKELNVGSMAHSTGKTMVNNVLSMDKDDVACFEKLRDLGV

SEQ ID NO: 129 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 130 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 131 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 132 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 133 - DNA, 828 bp, Streptococcus thermophilus
  source 1..828: mol_type = genomic DNA
atgtcagatatgtcaattatttctgcgattttggtcgtagctgttgccttccttgctggtcttgaaggtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 134 - protein, 275 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 135 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaatctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggcggcggaaatggtggcggaacttcatctggttcaggcgacccaatcggcgatatcttggaagactactag

SEQ ID NO: 136 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALCAIGVAIAFIYLNLSKQGGGNGGGNGGGTSSGSGDPIGDILEDY

SEQ ID NO: 137 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 138 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGSGMVVAVGYAMIINMMATREVWPFFAIGFALAAISQLTLIALCAIGVAIAFIYLNLSKQGGGNGGGNGGGTSSGSGDPIGDILEDY

SEQ ID NO: 139 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 140 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALGTIGVAIAFIYLNLSKQGGGNGGGNGGGTSSGSGDPIGDILEDY

SEQ ID NO: 141 - DNA, 828 bp, Streptococcus thermophilus
  source 1..828: mol_type = genomic DNA
atgtcagatatgtcaattatttctgcgattttggtcgtagctgttgccttccttgctggtcttgaaggtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 142 - protein, 275 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALGTIGVAIAFIYLNLSKQGGGNGGGNGGGTSSGSGDPIGDILEDY

SEQ ID NO: 143 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtactctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 144 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALGAIGVAIAFIYLNLSKQGGGNGGGNGGGTSSGSGDPIGDILEDY

SEQ ID NO: 145 - DNA, 828 bp, Streptococcus thermophilus
  source 1..828: mol_type = genomic DNA
atgtcagatatgtcaattatttctgcgattttggtcgtagctgttgccttccttgctggtcttgaaggtatccttgaccaattccaattccaccaaccacttgttgcatataccctcatcggtgctgccacaggtaacctcactgcaggtatcatgcttggtggttctcttcaaatgattacccttgcttgggcaaacatcggtgctgccgtagctcctgacgttgcccttgcatctgttgctgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccatcggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 146 - protein, 275 aa, Streptococcus thermophilus
MSDMSIISAILVVAVAFLAGLEGILDQFQFHQPLVAYTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATREVWPFFAIGFALAAISQLTLIALSTIGVAIAFIYLNLSKQGGGNGGGNGGGTSSGSGDPIGDILEDY

SEQ ID NO: 147 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatagtggcggaaatggtggcggaacttcatctggttcaggcgacccaatcggcgatatcttggaagactactag

SEQ ID NO: 148 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNSGGNGGGTSSGSGDPIGDILEDY

SEQ ID NO: 149 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattacctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctctttgtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 150 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASITFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALCAIGVAIAFIYLNLSKQGGGNGGGNGGGTSSGSGDPIGDILEDY

SEQ ID NO: 151 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctctctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctcttggtgccattggtgttgccatcgccttcatctacctcaacctttctaaacatggtggcggaaatggtggcggaaatggtggcggaacttcatctggttcaggcgacccaatcggcgatatcttggaagactactag

SEQ ID NO: 152 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATREVWPFFAIGFALAAISQLTLIALGAIGVAIAFIYLNLSKHGGGNGGGNGGGTSSGSGDPIGDILEDY

SEQ ID NO: 153 - DNA, 828 bp, Streptococcus thermophilus
  source 1..828: mol_type = genomic DNA
atgtcagatatgtcaattatttctgcgattttggtcgtagctgttaccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggtggaaatggtggcggaaatggtggcggaacttcatctggttcaggcgacccaatcggcgatatcttggaagactactag

SEQ ID NO: 154 - protein, 275 aa, Streptococcus thermophilus
MSDMSIISAILVVAVTFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNGGGNGGGTSSGSGDPIGDILEDY

SEQ ID NO: 155 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtagtggtatggtcgtagccgttggttacgccatgattatcaatatgatggctactcgtgaagtttggccattcttcgccatcggttttgctttggcagcaattagccaattgacacttatcgctcttggtgccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 156 - protein, 275 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATREVWPFFAIGFALAAISQLTLIALGAIGVAIAFIYLNLSKQGGGNGGGNGGGTSSGSGDPIGDILEDY

SEQ ID NO: 157 - DNA, 828 bp, Streptococcus thermophilus
  source 1..828: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactacctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgttaagtttggccattcttcgccattggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 158 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 159 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGGGMVVAVGYAMIINMMATR

SEQ ID NO: 160 - protein, 208 aa, Streptococcus thermophilus
MSDMSIISAILVVAVAFLAGLEGILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 161 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLSLLLQGLRIAVPAALLLAIPAQSVQHALGLMPDWLTHGLVVGSGMVVAVGYAMIINMMATR

SEQ ID NO: 162 - protein, 208 aa, Streptococcus thermophilus
MSDMSIISAILVVAVAFLAGLEGILDQFQFHQPLVAYTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGGGMVVAVGYAMIINMMATR

SEQ ID NO: 163 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATR

SEQ ID NO: 164 - protein, 208 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASITFVHAADKAAEHGNIAGVERAYYLSLLLQGLRIAVPAALLLAIPAQSVQHALGLMPDWLTHGLVVGGGMVVAVGYAMIINMMATR

SEQ ID NO: 165 - protein, 208 aa, Streptococcus thermophilus
MSDMSIISAILVVAVTFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYLALLLQGLRIAVPAALLLAIPAQSVQHALGLMPDWLTHGLVVGSGMVVAVGYAMIINMMATR

SEQ ID NO: 166 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 167 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 168 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgttttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactattcttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 169 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 170 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattattgcaagcttcttcggaatcatgtaa

SEQ ID NO: 171 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIIASFFGIM

SEQ ID NO: 172 - DNA, 912 bp, Streptococcus thermophilus
  source 1..912: mol_type = genomic DNA
atgtctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 173 - protein, 303 aa, Streptococcus thermophilus
MSEKIQLSQADRKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 174 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggttcatggaactatgaacgtatgcaaaacttgggttgggcttactcactcattcccgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcagctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaagtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaattacaatcatcatcggactctttgtagttggtattgttgcaagcttcttcggaatcatgtaa

SEQ ID NO: 175 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKSAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 176 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtattggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 177 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 178 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggtcttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataatattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 179 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMVFLWYTQELGYKAGSEITKDMSGGILKDITKGASILGMFILAVLVERWVSVVFTVKLPGKVLPKGAYIEWPKGYVTGDQLKTILGQVNDKLSFDNIQVDTLQKQLDSLIPGLTGLLLTFACMWLLKKKVSPITIIIGLFVVGIVASFFGIM

SEQ ID NO: 180 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagtttcttcggaatcatgtaa

SEQ ID NO: 181 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 182 - DNA, 912 bp, Streptococcus thermophilus
  source 1..912: mol_type = genomic DNA
atgtctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 183 - protein, 303 aa, Streptococcus thermophilus
MSEKIQLSQADRKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 184 - DNA, 912 bp, Streptococcus thermophilus
  source 1..912: mol_type = genomic DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggagttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattactggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 185 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNITGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 186 - DNA, 912 bp, Streptococcus thermophilus
  source 1..912: mol_type = genomic DNA
atggctgaaaaaattcaattatctcaagcggatcgtagaaaggtttggtggcgcttacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactctagaacttggttacaaagaaggttcagaaatcactaaagacatgtctggtggtatcttgaaagatattactaaaggggcatcaatacttggtatgttcttcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 187 - protein, 303 aa, Streptococcus thermophilus
MAEKIQLSQADRRKVWWRLQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNIAGPLIFFIGWNLIRMAFLWYTLELGYKEGSEITKDMSGGILKDITKGASILGMFFLAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 188 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccttcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 189 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAFLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 190 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttgaaattcttcaacactcacccttacgtagctgctcctatcatggggattaccttagctcttgaagaagaaaaagctaatggtactgaaatcgaagatgcggctatccaaggggttaaaatcggtatgatgggtccacttgccggtatcggtgaccctgtcttctggttcacaattcgtccaattctaggtgccattggtgcttcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcactaatcacaatcatcatcggactctttgtagttggtattgttgcaagcttcttcggaatcatgtaa

SEQ ID NO: 191 - protein, 303 aa, Streptococcus thermophilus
MAEKIQLSQADRKKVWWRSQFLQGSWNYERMQNLGWAYSLIPAIKKLYTNKEDQAAALKRHLKFFNTHPYVAAPIMGITLALEEEKANGTEIEDAAIQGVKIGMMGPLAGIGDPVFWFTIRPILGAIGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSLITIIIGLFVVGIVASFFGIM

SEQ ID NO: 192 - DNA, 912 bp, Streptococcus thermophilus
  source 1..912: mol_type = genomic DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggatcaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccccttgcaggtatcggtgaccctgtcttctggttcacaattcgtccaattcttggtgcccttggtgcatcattggcacaagctggtaacattactggtccacttatcttcttcattggttggaaccttatccgcatggccttcttgtggtacactcaagaacttggttacaaatcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 193 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQAGNITGPLIFFIGWNLIRMAFLWYTQELGYKSGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 194 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 195 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 196 - DNA, 912 bp, Streptococcus thermophilus
  source 1..912: mol_type = genomic DNA
atgtctgaaaaaattcaattatctcaagcggatcataaaaaggtttggtggcgctcacaattcttgcaaggttcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaatctggtaacattgctggtccacttatcttcttcattggttggaaccttatccgcatgaccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 197 - protein, 303 aa, Streptococcus thermophilus
MSEKIQLSQADHKKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGALGASLAQSGNIAGPLIFFIGWNLIRMTFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 198 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcatagaggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 199 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIEVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 200 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagatgctcctatcatgggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagtttcttcggaatcatgtaa

SEQ ID NO: 201 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVDAPIMGVTLALEEEKANGTEIEDAAIQGVKIGMMGPLAGIGDPVFWFTIRPILGALGASLAQAGNIAGPLIFFIGWNLIRMAFLWYTQELGYKAGSEITKDISGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 202 - DNA, 912 bp, Streptococcus thermophilus
  source 1..912: mol_type = genomic DNA
atggctgaaaaaattcaattatctcaagcggatcgtaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 203 - protein, 303 aa, Streptococcus thermophilus
MAEKIQLSQADRKKVWWRSQFLQGAWNYERMQNLGWAYSLIPAIKKLYTNKEDQAAALKRHLEFFNTHPYVAAPIIGVTLALEEEKANGTEIEDAAIQGVKIGMMGPLAGIGDPVFWFTIRPILGALGASLAQAGNIAGPLIFFIGWNLIRMAFLWYTQELGYKAGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 204 - DNA, 912 bp, Streptococcus thermophilus
  source 1..912: mol_type = genomic DNA
atggctgaaaaaattcaattatctcaagcggatcgtagaaaggtttggtggcgctcacaattcttgcaaggttcatggaactatgaacgtatgcaaaacttgggttgggcttactcactcattcctgctatcaaaaaactttatactaacaaagaggatcaagccgcagctcttaaacgtcacttggaattcttcaacactcacccttacgtagctgctcctatcatgggggttaccttagctcttgaagaagaaaaagctaatggtactgaaatcgaagatgcggctatccaaggggttaaaatcggtatgatgggtccacttgcaggtatcggtgaccctgtcttctggttcacaattcgtccaattcttggtgccattggtgcttcattggcacaagctggtaacattgctggtccacttatcttcttcattggttggaaccttatccgcatggccttcttgtggtacactcaagaacttggttacaaatcaggttcagaaatcactaaagacatgtctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgatcaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 205 - protein, 303 aa, Streptococcus thermophilus
MAEKIQLSQADRRKVWWRSQFLQGSWNYERMQNLGWAYSLIPAIKKLYTNKEDQAAALKRHLEFFNTHPYVAAPIMGVTLALEEEKANGTEIEDAAIQGVKIGMMGPLAGIGDPVFWFTIRPILGAIGASLAQAGNIAGPLIFFIGWNLIRMAFLWYTQELGYKSGSEITKDMSGGILKDITKGASILGMFILAVLVERWVSVVFTVKLPGKVLPKGAYIEWPKGYVTGDQLKTILGQVNDKLSFDKIQVDTLQKQLDSLIPGLTGLLLTFACMWLLKKKVSPITIIIGLFVVGIVASFFGIM

SEQ ID NO: 206 - DNA, 913 bp, Streptococcus thermophilus
  source 1..913: mol_type = genomic DNA
atggctgaaaaaattcaattatctcaagcggatcgtaaaaaaggtttggtggcgctcacaattcttgcaaggtgcatggaactatgaacgtatgcaaaacttgggttgggcttactcactcattcctgctatcaaaaaactttatactaacaaagaggaccaagccgcagctcttaaacgtcacttggaattcttcaacactcacccttacgtagctgctcctatcataggggttaccttagctcttgaagaagaaaaagctaatggtactgaaatcgaagatgcggctatccaaggggttaaaatcggtatgatgggtccacttgccggtatcggtgaccctgtcttctggttcacaattcgtccaattcttggtgcccttggtgcatcattggcacaagctggtaacattgctggtccacttatcttcttcattggttggaaccttatccgcatggccttcttgtggtacactcaagaacttggttacaaagcaggttcagaaatcactaaagacatatctggtggtatcttgaaagatattactaaaggggcatcaatacttggtatgttcatcttggccgtcctcgttgaacgttgggtatctgtcgtcttcactgtaaagcttccaggtaaagttttgcctaaaggtgcttatattgaatggccaaaaggatatgttactggtgaccaactaaaaactatccttggtcaagtcaacgataagcttagctttgataagattcaagtcgataccctacaaaaacaattggattcattaattccaggtttgacgggacttctccttacttttgcatgtatgtggttgcttaagaagaaagtttcaccaatcacaatcatcatcggactctttgtagttggtattgttgcaagcttcttcggaatcatgtaa

SEQ ID NO: 207 - protein, 28 aa, Streptococcus thermophilus
MAEKIQLSQADRKKGLVALTILARCMEL

SEQ ID NO: 208 - protein, 28 aa, Streptococcus thermophilus
MAEKIQLSQADRKKGLVALTILARFMEL

SEQ ID NO: 209 - protein, 28 aa, Streptococcus thermophilus
MSEKIQLSQADRKKGLVALTILARFMEL

SEQ ID NO: 210 - protein, 28 aa, Streptococcus thermophilus
MAEKIQLSQADRRKGLVALTILARFMEL

SEQ ID NO: 211 - protein, 28 aa, Streptococcus thermophilus
MSEKIQLSQADHKKGLVALTILARFMEL

SEQ ID NO: 212 - DNA, 159 bp, Streptococcus thermophilus
  source 1..159: mol_type = genomic DNA
atggattacaaacaaattgcaaaagaagtcatcgaagccctcggtggacgtgaaaatgttaacagtgttgctcactgtgcgacacgtctacgtgttatggttaaagatgaaaacaaaatcaataaagaaaaaagctgagaatattgaaaaagttcaagg

SEQ ID NO: 213 - DNA, 248 bp, Streptococcus thermophilus
  source 1..248: mol_type = genomic DNA
atttaataatctcctaatttatttattagtaatataggaaacgttttacctagaaaaatcaacgatttataataagaaaacaaaaaaatgctaaacgtttgacatatgacagaaagatgttaaaattaatatcgtaaagaaaagtgaaacgttttcaaaaacaaattttgttaaagatgttaaaattgatatcgtaaagaaaagcgaaacgttttcaaaaacaaattttattaaggagaattttgcaa

SEQ ID NO: 214 - DNA, 158 bp, Streptococcus thermophilus
  source 1..158: mol_type = genomic DNA
atggattacaaacaaattgcaaaagaagtcatcgaagccctcggtggacgtgaaaatgttaacagtgttgctcactgtgcgacacgtctacgtgttatggttaaagatgaaaacaaaatcaataaagaaaaagctgagaatattgaaaaagttcaagg

SEQ ID NO: 215 - DNA, 248 bp, Streptococcus thermophilus
  source 1..248: mol_type = genomic DNA
atttaataatctcctaatttatttattagtaatatagtaaacgttttacctagaaaaatcaacgatttataataagaaaacaaaaaaatgctaaacgtttgacatatgacagaaagatgttaaaattaatatcgtaaagaaaagtgaaacgttttcaaaaacaaattttgttaaagatgttaaaattgatatcgtaaagaaaagcgaaacgttttcaaaaacaaattttattaaggagaattttgcaa

SEQ ID NO: 216 - DNA, 175 bp, Streptococcus thermophilus
  source 1..175: mol_type = genomic DNA
ccaaatggagaaccgcctcaaatgataaatttattattcaacaccagttgacgtagaaccacagttatggttcttgtgtatttttttatctcttgttttttcccgaaataagagtaatatagagctatgcttaattttttaggtaaaaaattattttaaagaggtaaatataaac

SEQ ID NO: 217 - DNA, 176 bp, Streptococcus thermophilus
  source 1..176: mol_type = genomic DNA
ccaaatggagaaccgcctcaaatgataaatttattattcaacaccagttgacgtagaaccacagttatggttcttgtgtatttttttatctcttgtttttttcccgaaataagagtaatatagagctatgcttaattttttaggtaaaaaattattttaaagaggtaaatataaac

SEQ ID NO: 218 - DNA, 176 bp, Streptococcus thermophilus
  source 1..176: mol_type = genomic DNA
ccaaatggagaaccgcctcaaatgataaatttattattcaacaccagttgacgtagaaccacagttatggttcttgtgtatttttttatctcttgttttttccccgaaataagagtaatatagagctatgcttaattttttaggtaaaaaattattttaaagaggtaaatataaac

SEQ ID NO: 219 - DNA, 175 bp, Streptococcus thermophilus
  source 1..175: mol_type = genomic DNA
ccaaatggagaaccgcctcaaatgataaatttattattcaacaccagttgacgtagaaccacagttatggttcttgtgtatttttttatctcttgctttttcccgaaataagagtaatatagagctatgcttaattttttaggtaaaaaattattttaaagaggtaaatataaac

SEQ ID NO: 220 - DNA, 1496 bp, Streptococcus thermophilus
  source 1..1496: mol_type = genomic DNA
ccaaatggagaaccgcctcaaatgataaatttattattcaacaccagttgacgtagaaccacagttatggttcttgtgtatttttttatctcttgttttttcccgaaataggtagtgtaaaataagttgtgtaaacacaaaaaggaataaatccgttatagtagagttgcaaaacattactagaaagagatttattcctatgactccgtttaccacagaactacttaacttccttgcccaaaagcaagatattgatgaatttttccgtacttctcttgaaacagctatgaatgatctgcttcaagcagagttatcagcctttttagggtatgaaccttacgataaattaggctataattctgggaatagtcgtaacggaagctatgcacggaaattcgaaaccaaatatgggactgttcagttgagtattcctagagatcgtaatgggaactttagtccagctttgcttcccgcttatggacgtcgagatgaccacttggaagagatggttatcaaactctatcaaaccggtgtaacgactcgagaaattagtgatatcatcgagcgaatgtatggtcatcactatagtcctgccacaatttctaatatctcaaaagcaactcaggagaatgtcgctacttttcatgagcgaagcttagaagccaattactctgttttatttcttgacggaacctatcttcccttaagacgtggaaccgttagtaaagaatgtattcatatcgcacttggcattacaccagaaggacagaaggctgttcttggatatgaaatcgctccaaatgaaaacaatgcttcttggtccaccctgttagacaagcttcaaaaccaaggaatccaacaggtttctcttgtagtgaccgatggcttcaaggggcttgaagagattatcaatcaggcttacccattagctaaacaacaacgttgcttaattcatattagtcgaaatctagctagtaaagtgaaacgagcagatagagcggttattctggagcaatttaaaacgatttatcgtgctgaaaatttagaaatggcagtgcaagctttagagaactttatcgccgaatggaaaccaaagtataggaaagtcatggaaagtctggagaatacggataatcttttaactttttatcagtttccctaccagatttggcatagcatttattcgacaaacctcattgagtctcttaacaaagaaatcaaacgtcaaacgaaaaagaaggtcctttttcctaacgaggaggctctggaacgttatttagttaccctgtttgaagattataatttcaagcaaaatcaacgcatccataaagggtttggacaatgtactgacacacttgaaagcttatttgattaacattcttcaactctacttgagtgtttacacataattattgacagtatcccgaaataagagtaatatagagctatgcttaattttttaggtaaaaaattattttaaagaggtaaatataaac

SEQ ID NO: 221 - DNA, 1321 bp, Streptococcus thermophilus
  source 1..1321: mol_type = genomic DNA
ggtagtgtaaaataagttgtgtaaacacaaaaaggaataaatccgttatagtagagttgcaaaacattactagaaagagatttattcctatgactccgtttaccacagaactacttaacttccttgcccaaaagcaagatattgatgaatttttccgtacttctcttgaaacagctatgaatgatctgcttcaagcagagttatcagcctttttagggtatgaaccttacgataaattaggctataattctgggaatagtcgtaacggaagctatgcacggaaattcgaaaccaaatatgggactgttcagttgagtattcctagagatcgtaatgggaactttagtccagctttgcttcccgcttatggacgtcgagatgaccacttggaagagatggttatcaaactctatcaaaccggtgtaacgactcgagaaattagtgatatcatcgagcgaatgtatggtcatcactatagtcctgccacaatttctaatatctcaaaagcaactcaggagaatgtcgctacttttcatgagcgaagcttagaagccaattactctgttttatttcttgacggaacctatcttcccttaagacgtggaaccgttagtaaagaatgtattcatatcgcacttggcattacaccagaaggacagaaggctgttcttggatatgaaatcgctccaaatgaaaacaatgcttcttggtccaccctgttagacaagcttcaaaaccaaggaatccaacaggtttctcttgtagtgaccgatggcttcaaggggcttgaagagattatcaatcaggcttacccattagctaaacaacaacgttgcttaattcatattagtcgaaatctagctagtaaagtgaaacgagcagatagagcggttattctggagcaatttaaaacgatttatcgtgctgaaaatttagaaatggcagtgcaagctttagagaactttatcgccgaatggaaaccaaagtataggaaagtcatggaaagtctggagaatacggataatcttttaactttttatcagtttccctaccagatttggcatagcatttattcgacaaacctcattgagtctcttaacaaagaaatcaaacgtcaaacgaaaaagaaggtcctttttcctaacgaggaggctctggaacgttatttagttaccctgtttgaagattataatttcaagcaaaatcaacgcatccataaagggtttggacaatgtactgacacacttgaaagcttatttgattaacattcttcaactctacttgagtgtttacacataattattgacagtatcccgaaata

SEQ ID NO: 222 - DNA, 829 bp, Streptococcus thermophilus
  source 1..829: mol_type = genomic DNA
atgtcagatatgtcaattatttctgcgattttggtcgtagctgttgccttccttgctggtcttgaaagtatccttgaccaattccaattccaccaaccacttgttgcatgtaccctcatcggtgctgccacaggtaacctcactgcaggtatcatgcttggtggttctcttcaaatgattacccttgcttgggcaaacatcggtgctgccgtagctcctgacgttgcccttgcatctgttgccgctgccatcattttggttaaaggtggtaaatttacagctgaaggtatcggtgttgcgattgcaatagctatcctgcttgcagttgcaggtctcttcctaactatgcctgttcgtacagcatctattgcctttgttcatgctgcagataaagctgcagaacacggaaacatcgctggtgttgaacgtgcatactaccctcgctctccttcttcaaggtttgcgtattgctgtgccagcagcccttcttcttgccatcccggcccaatctgttcaacatgcccttggcttgatgcctgactggctcacccatggtttggttgtcggtggtggtatggtcgtagccgttggttacgccatgattatcaatatgatggctactcgtgaagtttggccattcttcgccattggttttgctttggcagcaattagccaattgacacttatcgctcttagtaccattggtgttgccatcgccttcatctacctcaacctttctaaacaaggtggcggaaatggtggcggaaatggtggcggaacttcatctggttcaggcgacccaatcggcgatatcttggaagactactag

SEQ ID NO: 223 - protein, 179 aa, Streptococcus thermophilus
MSDMSIISAILVVAVAFLAGLESILDQFQFHQPLVACTLIGAATGNLTAGIMLGGSLQMITLAWANIGAAVAPDVALASVAAAIILVKGGKFTAEGIGVAIAIAILLAVAGLFLTMPVRTASIAFVHAADKAAEHGNIAGVERAYYPRSPSSRFAYCCASSPSSCHPGPICSTCPWLDA

SEQ ID NO: 224 - DNA, 409 bp, Streptococcus thermophilus
  source 1..409: mol_type = genomic DNA
catatttaataatctcctaatttatttattagtaatatagtaaacgttttacctagaaaaatcaacgatttataataagaaaacaaaaaaatgctaaacgtttgacatatgacagaaagatgttaaaattaatatcgtaaagaaaagtgaaacgttttcaaaaacaaattttgttaaagatgttaaaattgatatcgtaaagaaaagcgaaacgttttcaaaaacaaattttattaaggagaattttgcaaatggattacaaacaaattgcaaaagaagtcatcgaagccctcggtggacgtgaaaatgttaacagtgttgctcactgtgcgacacgtctacgtgttatggttaaagatgaaaacaaaatcaataaagaaaaagctgagaatattgaaaaagttcaagg

SEQ ID NO: 225 - DNA, 199 bp, Streptococcus thermophilus
  source 1..199: mol_type = genomic DNA
ccaaatggagaaccgcctcaaatgataaatttattattcaacaccagttgacgtagaaccacagttatggttcttgtgtatttttttatctcttgttttttcccgaaataagagtaatatagagctatgcttaattttttaggtaaaaaattattttaaagaggtaaatataaacatgcaaggagttcttttcgcgctt

(19) United States
(12) Patent Application Publication
(10) Pub. No.: US 2025/0171730 A1
(43) Pub. Date: May 29, 2025

(54) NEW LACTIC ACID BACTERIA

(71) Applicant: INTERNATIONAL N&H DENMARK APS, Kongens Lyngby (DK)

(72) Inventors: Armelle Cochu-Blachère, Naintrè (FR); Elodie Guillou, Dangè-Saint-Romain (FR); Christophe Fremaux, Dangè-Saint-Romain (FR); Anne-Claire Coute-Monvoisin, Dangè-Saint-Romain (FR)

(21) Appl. No.: 18/843,592

(22) PCT Filed: Mar. 2, 2023
(86) PCT No.: PCT/EP2023/055363

§ 371 Date: Sep. 3, 2024

(30) Foreign Application Priority Data
Mar. 3, 2022 (EP) 22159955.8

(51) Int. Cl. C12N 1/20; A23C 9/123; C07K 14/315; C12N 9/12; C12Q 1/14; C12R 1/46

(52) U.S. Cl. CPC C12N 1/20; A23C 9/1238; C07K 14/315; C12N 9/1205; C12Q 1/14; C12R 2001/46; C12Y 207/01002

(57) ABSTRACT

The present invention relates to lactose-positive, sucrose-negative, Streptococcus thermophilus strains. The strains are carrying one or more mutation in one or more gene of the sucrose regulon and optionally one or more further mutation affecting the glucose porter, such as affecting glcU and/or its expression. The strains of the present invention may further carry mutations in genes encoding a protein of the mannose-glucose-specific PTS and a glucokinase, a protein of the mannose-glucose-specific PTS and the catabolite control protein A (CcpA), and/or a protein of the mannose-glucose-specific PTS, a glucokinase and CcpA. The inventive strains may, when fermenting milk do this without the need or with a need for adding a reduced amount of sucrose, optionally providing a low-lactose fermented milk and/or a fermented milk not undergoing post-acidification when stored at fermentation temperature. The invention also concerns a composition comprising at least one, lactose-positive, sucrose-negative, Streptococcus thermophilus strain of the invention, and the use of this strain or composition to manufacture a fermented dairy product.

### FIELD OF THE INVENTION

The present invention relates to lactose-positive, sucrose-negative, Streptococcus thermophilus strains. The strains are carrying one or more mutation in one or more gene of the sucrose regulon and optionally one or more further mutation affecting the glucose porter (non-PTS glucose permease), such as affecting glcU and/or its expression. The strains of the present invention may further carry mutations in genes encoding a protein of the mannose-glucose-specific PTS and a glucokinase, a protein of the mannose-glucose-specific PTS and the catabolite control protein A (CcpA), and/or a protein of the mannose-glucose-specific PTS, a glucokinase and CcpA.

The inventive strains may, when fermenting milk containing any sucrose, do this without consuming this sucrose, and thus provide for a reduced need for added sucrose, or with a need for adding a reduced amount of sucrose, while still obtaining the expected level of sweetness from sucrose. Optionally the inventive strains provide for a low-lactose fermented milk and/or a fermented milk not undergoing post-acidification when stored at fermentation temperature. The invention also concerns a composition comprising at least one, lactose-positive, sucrose-negative, Streptococcus thermophilus strain of the invention, and the use of this strain or composition to manufacture a fermented dairy product.

### BACKGROUND TO THE INVENTION

The food industry uses bacteria in order to improve the taste and the texture of food or feed products. In the case of the dairy industry, lactic acid bacteria are commonly used in order to, for example, bring about the acidification of milk (by fermentation of lactose) and to texturize the product into which they are incorporated. For example, the lactic acid bacteria of the species Streptococcus thermophilus (S. thermophilus) are used extensively, alone or in combination with other bacteria, in the manufacture of fresh fermented dairy products, such as cheese or yoghurt.

One of the challenges in the use of lactic acid bacteria in dairy technology is post-acidification, i.e. the production of lactic acid by the lactic acid bacteria after the target pH (the one required by the technology) has been obtained [termination of fermentation]. Thus, the post-acidification phenomenon is not only an issue for the dairy product manufacturers (who would like to have flexible manufacturing process, without necessarily having a rapid cooling step right after the target pH is obtained) but also for the consumers (production of lactic acid leading to an elevated acidity and reduced quality of the fermented product).

Another challenge in the dairy industry is that cultures may be consuming any added or already present sucrose during fermentation. To achieve an expected sweetness of fermented products, it is necessary to add sucrose to the milk, prior, during or at the end of fermentation. Another challenge in the dairy industry is that cultures may be consuming any added sucrose during fermentation. Thus, to achieve the expected sweetness, it may be necessary to add an excess of sucrose into the milk. In addition, it could be envisioned to reduce the amount of added sucrose even more by releasing part or all of the glucose moiety resulting from lactose hydrolysis by the culture.

In addition, there is a trend from dairy consumers to have fermented products with reduced or low content of lactose (lactose intolerance).

Therefore, there is a need for improving methods for producing fermented dairy products, which are both satisfactory for the manufacturers and the consumers, not undergoing post-acidification with a reduced need for addition of sucrose and with a reduced lactose content.

### DESCRIPTION OF THE DRAWINGS

FIG. 1: DNA sequence alignment of the chromosomal locus of strain DGCC8368 bearing the scrA₈₂₉^{FS} and pscrB₈₂₉ mutations with that of ST1. Nucleotide identical in both sequences are indicted by dots in the sequence of DGCC8368. Differences are indicated with nucleotide in bold in the sequence of DGCC8368 and named. Difference pscrB₈₂₉ correspond to the replacement of a T nucleotide by a G nucleotide in position 36 of the sequence; difference scrA₈₂₉^{FS} corresponds to the insertion of a A nucleotide in position 384 of the sequence. Start codons are in italics underlined, open reading frames are in italics, and putative minus 10-box is in bold-underlined.

FIG. 2: DNA sequence alignment of the promoter region of the glcU gene of strain ST1 with that of various sucrose-negative, glucose-positive mutants described in example 5. Nucleotide identical in all sequences are indicated by dots in the sequence of mutants. Sequence differences are named and indicated with nucleotide in bold in the sequence of the mutants or by a black triangle for the insertion of IS1191. Mutation pglcU^{SNP_C} corresponds to the replacement of a T nucleotide by a C nucleotide in position 96 of the sequence; differences pglcU^{IN_T} and pglcU^{IN_C} correspond to the insertion of a T or a C nucleotide in position 102 of the sequence. Mutation pglcU^{IS} corresponds to the insertion of IS1191 between position 111 and 112 of the sequence (or between position 110 and 111 of the sequence of e.g. ST1 and ST1-ABU4). Start codons are in italics underlined and open reading frames are in italics.

FIG. 3: Comparison of the μ_{glucose}/μmax_{lactose} ratio calculated for: wildtype (parental) strains (black) their respective scrA₈₂₉^{FS}-pscrB₈₂₉ mutants (white) and their glucose-positive derivatives bearing mutations within pglcU (grey).

FIGS. 4 to 8: Graphs representing the evolution of the pH over time (A) and representing the velocity as a function of the pH (B) of a plain milk (1) and milk supplemented with 7% (w/v) sucrose (2) fermented with ST1 (FIG. 4), ST1-GM (FIG. 5), ST1-GM-AB (FIG. 6), ST1-GM-ABU1 (FIG. 7), ST1-CM-ABU1 (FIG. 8).

### DETAILED DESCRIPTION

Starter cultures of Streptococcus thermophilus used in the art will normally consume an amount of any sucrose added in milk for sweetening purpose during yoghurt fermentation; with the following consequences:

a) it is required to add more sucrose than (theoretically) necessary to achieve a certain level of sweetness because the strain is using it (instead of using lactose).

b) the functionalities of a controlled-pH phenotype resulting from fermentation with S. thermophilus mutated in manM (and/or manL and/or manN) and in glcK and/or in ccpA (as described in the examples of WO2019197051) would be reduced or lost in sweet milk.

c) the release of glucose resulting from fermentation of S. thermophilus mutated in only glcK and/or in ccpA or in glcK and/or in ccpA and in manM (and/or manL and/or manN) (as described in the examples of WO2019122365 and WO2019197051) may be reduced or absent in sweet milk.

d) the ability to overconsume lactose by fermentation of S. thermophilus mutated in glcK, ccpA, glcK/ccpA, glcK/manM (manL/manN), ccpA/manM (manL/manN) and glcK/ccpA/manM (manL/manN) (as described in the examples of WO2019122365 and WO2019197051) is reduced or absent in sweet milk.

The present invention has addressed each of these problems in the art providing solutions in terms of specific mutations modifying the sugar metabolism, which mutations may be used in the design of improved Streptococcus thermophilus strains, which again can be used to obtain low lactose fermented milk products and/or which can be used to produce fermented milk containing sucrose not undergoing post-acidification even when stored at fermentation temperature and without challenges described above.

Accordingly, in a first aspect the present invention relates to a lactose-positive, sucrose-negative, Streptococcus thermophilus strain carrying one or more mutations selected from the group consisting of:
1) one or more mutation in one or more gene of the sucrose regulon; which mutation provides for a significant reduction in the ability to grow on and/or transport sucrose by said strain; and which mutation is in
   i) one or more gene of the sucrose regulon, such as the scrA gene (encoding for the phosphoenolpyruvate-sugar phosphotransferase system (PTS) sucrose-specific EIIABC^{Sucrose} component), and/or the scrB gene (encoding for the sucrose-6-phosphate hydrolase) and/or the scrR gene (encoding for a putative sucrose operon regulator), and
   ii) optionally a further mutation in the promoter region of the sucrose regulon regulating the expression of the scrA, scrB and/or scrR genes, such as a mutation that protects the strain from reversing from the sucrose negative phenotype; and/or
2) optionally one or more further mutation affecting the glucose porter, such as affecting glcU and/or its expression, which mutation restores or improves glucose consumption of said strain.

It is to be understood that in some embodiments, the lactose-positive, sucrose-negative, Streptococcus thermophilus strain according to the present invention has a mandatory mutation in one or more of the scrA gene, and/or the scrB gene and/or the scrR gene. In some embodiments the lactose-positive, sucrose-negative, Streptococcus thermophilus strain according to the present invention has a further mutation in either a) the promoter region regulating the expression of the scrA, scrB and/or scrR genes, such as a mutation that protect the strain from reversing from the sucrose negative phenotype; or
b) one or more further mutation affecting the glucose porter, such as affecting glcU and/or its expression, which mutation restores or improves glucose consumption of said strain when grown on glucose as the sole source of carbohydrates; or both a) and b).

In a second aspect the present invention relates to a composition comprising at least one, in particular one, lactose-positive, sucrose-negative, Streptococcus thermophilus strain according to the invention, in particular in combination with another lactic acid bacteria, in particular with one or more strain(s) selected from the group consisting of a strain of the Lactobacillus genus, such as a Lactobacillus delbrueckii subsp bulgaricus strain, a strain of the Lactococcus genus, such as a Lactococcus lactis strain or a strain of the Bifidobacterium genus.

In a third aspect the present invention relates to a method for manufacturing a fermented dairy product, in particular a fermented milk, comprising inoculating a milk substrate with the lactose-positive, sucrose-negative, Streptococcus thermophilus strain according to the invention, or a composition according to the invention, and fermenting said inoculated milk, to obtain a fermented dairy product.

In a further aspect the present invention relates to the use of the lactose-positive, sucrose-negative, Streptococcus thermophilus strain according to the invention, or a composition according to the invention described above, to manufacture a fermented dairy product.

In a further aspect the present invention relates to a fermented dairy product comprising at least one, in particular one, lactose-positive, sucrose-negative, Streptococcus thermophilus strain according to the invention, or as obtained by a method according to the invention described above.

In a further aspect the present invention relates to a Streptococcus thermophilus strain, such as a lactose-positive, sucrose-negative Streptococcus thermophilus strain selected from the group consisting of:
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34132;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34134;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34138;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34139;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34140;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34146;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34147;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 16 Feb. 2022 under number DSM34172.

In a further aspect the present invention relates to a lactose-positive, sucrose-negative, Streptococcus thermophilus strain selected from the group consisting of:
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇); and optionally further comprising a ccpA sequence of SEQ ID NO: 71 or a ccpA variant sequence having at least 95% identity with SEQ ID NO: 71.
a strain of a Streptococcus thermophilus, such as the strain deposited under the Budapest Treaty on Jan. 14, 2014 in the name of Danisco Deutschland GmbH at Leibniz-Institut DSMZ under number DSM28255 [herein referred to as ST4 or DSM28255 strain] comprising one or more of the following features:
   1) into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇);
   6) having a ccpA sequence of SEQ ID NO:71 or a ccpA variant sequence having at least about 90%, such as at least 95% identity with SEQ ID NO: 71.

In a further aspect the present invention relates to a lactose-positive, sucrose-negative, Streptococcus thermophilus strain selected from the group consisting of:
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of the scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO:25, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇); and optionally further comprising a ccpA sequence of SEQ ID NO:71 or a ccpA variant sequence having at least about 90%, such as at least 95% identity with SEQ ID NO:71.
a strain of a Streptococcus thermophilus, such as the strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO: 213 (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇);
   6) having a ccpA sequence of SEQ ID NO:71 or a ccpA variant sequence having at least about 90%, such as at least 95% identity with SEQ ID NO: 71.

In a further aspect the present invention relates to a lactose-positive, sucrose-negative, Streptococcus thermophilus strain selected from the group consisting of:
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of the scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glCK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇); and optionally further comprising a ccpA sequence of SEQ ID NO:71 or a ccpA variant sequence having at least about 90%, such as at least 95% identity with SEQ ID NO:71.
a strain of a Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glCK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇);
   6) having a ccpA sequence of SEQ ID NO:71 or a ccpA variant sequence having at least about 90%, such as at least 95% identity with SEQ ID NO:71.

In a further aspect the present invention relates to a lactose-positive, sucrose-negative, Streptococcus thermophilus strain selected from the group consisting of:
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 (pglcU^{SNP_C}, pglcU^{IN_T}, pglcU^{IN_C}, pglcU^{IS});
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of the scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glCK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇); and optionally further comprising a ccpA sequence of SEQ ID NO: 71 or a ccpA variant sequence having at least about 90%, such as at least 95% identity with SEQ ID NO:71.
a strain of a Streptococcus thermophilus strain deposited under accession number DSM34172 (herein referred to as ST1 or DSM34172) on Sep. 29, 2020, at the DSMZ [Deutsche Sammlung von Mikroorganismen und Zellkulturen GmbH, Inhoffenstrasse 7B, D-38124 Braunschweig-Germany] comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO:213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇);
   6) having a ccpA sequence of SEQ ID NO:71 or a ccpA variant sequence having at least about 90%, such as at least 95% identity with SEQ ID NO: 71.

In a further aspect the present invention relates to a lactose-positive, sucrose-negative, Streptococcus thermophilus strain selected from the group consisting of:
a strain of Streptococcus thermophilus strain into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS});
a strain of Streptococcus thermophilus into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain of Streptococcus thermophilus into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 (pglcU^{SNP_C}, pglcU^{IN_T}, pglcU^{IN_C}, pglcU^{IS});
a strain of Streptococcus thermophilus into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain of Streptococcus thermophilus into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain of Streptococcus thermophilus into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇); and optionally further comprising a ccpA sequence of SEQ ID NO: 71 or a ccpA variant sequence having at least about 90%, such as at least 95% identity with SEQ ID NO: 71.
a strain of Streptococcus thermophilus comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO:213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇);
   6) having a ccpA sequence of SEQ ID NO:71 or a ccpA variant sequence having at least about 90%, such as at least 95% identity with SEQ ID NO:71.

In a further aspect the present invention relates to a method for the selection of a lactose-positive, sucrose-negative, Streptococcus thermophilus strain, which method includes the steps of:
1) providing a strain of Streptococcus thermophilus including
   a) one or more mutation in one or more gene of the sucrose regulon; which mutation provides for a significant reduction in the ability to grow on and/or transport sucrose by said strain; and which mutation at least comprises
      i) one or more of the scrA gene, and/or the scrB gene and/or the scrR gene, and
      ii) optionally a further mutation in the promoter region regulating the expression of the scrA, scrB and/or scrR genes, such as a mutation that protect the strain from reversing from the sucrose negative phenotype;
2) screen for a strain of Streptococcus thermophilus from step 1), which strain has a significant reduction in the ability to grow on and transport sucrose by said strain; and
3) optionally screen prior to, simultaneously with, or after any one of steps 1 and 2 for a Streptococcus thermophilus strain which has a restored or improved glucose consumption, optionally from one or more further mutation affecting the glucose porter, such as affecting glcU and/or its expression.

In a particular embodiment, a “variant sequence” of the strain as defined herein has a sequence identity of at least 90%, or at least 95% with reference to a specific SEQ ID NO referred to, or with the genome sequence of the parent strain from which the variant sequence is obtained including or without including the specific mutation or insertion of the strain, in particular an identity of at least 90%, at least 91%, at least 95%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.94%, at least 99.96%, at least 99.98%, or at least 99.99% with the specific SEQ ID NO referred to, or with the genome sequence of the parent strain from which the variant is obtained. The identity is described in comparing the two sequences over their full-length (global alignment) and may be calculated using any program based on the Needleman-Wunsch algorithm.

In dairy application, it is expected for the Streptococcus thermophilus strains of the invention to not consume sucrose, i.e. to have a sucrose-negative phenotype. Accordingly, as used herein “sucrose-negative” when referring to the phenotype of a strain within the scope of the present invention is meant a strain with very limited or no ability to grow on a medium containing sucrose as a sole source of carbohydrate. This is measured by Test A described herein. In the present context a Streptococcus thermophilus strain with very limited or no ability to grow on a medium containing sucrose as a sole source of carbohydrate refers to a strain wherein ratio of colonies forming (cfu) on sucrose (sucrose-positive colonies) compared to colonies forming (cfu) on lactose is below 10⁻², such as below 10⁻³, 10⁻⁴, 10⁻⁵, 10⁻⁶, or 10⁻⁷ when tested with Test A described herein. As used herein a mutation that “provides for a significant reduction in the ability to grow on and transport sucrose” is a mutation in a strain that induces or helps to induce this strain to get a “sucrose-negative phenotype”.

A Streptococcus thermophilus strain is considered “sucrose-positive phenotype” when its ratio of colonies forming (cfu) on sucrose (sucrose-positive colonies) compared to colonies forming (cfu) on lactose are close to 1, or higher than 10⁻² when tested with Test A described herein.

Alternatively, enzymatic means may be considered for ability of strains to grow on sucrose. For example, by measuring the ability of resting cells of a scrA mutant to transport sucrose; or the ability of an intracellular extract of a scrB mutant to hydrolyze phosphorylated sucrose (sucrose-6-Phosphate).

The present inventors have shown that by mutating selected genes of the sucrose regulon in Streptococcus thermophilus optionally in combination with one or more mutation affecting the glucose porter, such as affecting glcU and/or its expression, then the Streptococcus thermophilus strains will display a “sucrose-negative phenotype” defined above and an optional glucose-positive phenotype.

In some embodiments, the present invention is directed to a lactose-positive, sucrose-negative, Streptococcus thermophilus strain further carrying a mutation in genes selected from the group consisting of 1) a least one gene encoding a protein of the mannose-glucose-specific PTS and a glcK gene, 2) at least one gene encoding a protein of the mannose-glucose-specific PTS and a ccpA gene, and 3) a gene encoding a protein of the mannose-glucose-specific PTS, a glcK gene and a ccpA gene, as described in the examples of WO2019122365 and WO2019197051 patents.

In some embodiments, the lactose-positive, sucrose-negative, Streptococcus thermophilus strain is carrying a mutation in one gene encoding a protein of the mannose-glucose-specific PTS and a glcK gene. In an embodiment, the gene encoding a protein of the mannose-glucose-specific PTS is selected from the group consisting of the manL gene, the manM gene and the manN gene and the manO gene. In an embodiment, the gene encoding a protein of the mannose-glucose-specific PTS is selected from the group consisting of the manL gene, the manM gene and the manN gene. In an embodiment, the lactose-positive, sucrose-negative, Streptococcus thermophilus strain is carrying a mutation in one gene selected from the group consisting of manL gene, the manM gene and the manN gene. In an embodiment, the lactose-positive, sucrose-negative, Streptococcus thermophilus strain is carrying a mutation in 2 genes selected from the group consisting of manL gene, the manM gene and the manN gene. In an embodiment, the lactose-positive, sucrose-negative, Streptococcus thermophilus strain is carrying a mutation in the manL gene, the manM gene and the manN gene. In some embodiments, the lactose-positive, sucrose-negative, Streptococcus thermophilus strain is carrying a mutation in only one gene encoding a protein of the mannose-glucose-specific PTS being the manM gene, as described in the examples of WO2019122365 and WO2019197051.

Another aspect of the lactose-positive, sucrose-negative, Streptococcus thermophilus strains according to the present invention is a release of glucose during milk fermentation. As described herein, the lactose-positive, sucrose-negative, Streptococcus thermophilus strains of the invention can be further characterized by their ability to release glucose when used to ferment milk. This ability is defined herein by the concentration of glucose, which is released into the milk, when the strain of the invention is used to ferment milk. In an embodiment, the fermentation conditions are according to, and the concentration of glucose released is determined as described in example 6. Accordingly, in some embodiments, the lactose-positive, sucrose-negative, Streptococcus thermophilus strain according to the present invention is releasing a concentration of glucose assayed as described in example 6, which is at least 50 mM, such as at least 60, 70, 80 or 90 mM or releasing a concentration of glucose which is increased of at least 150% or at least 200%, such as 300%, 400%, 500% as compared to the glucose concentration released by the corresponding parent strain not having the described mutations of the invention, when both assayed as described in example 6. In some embodiments, a mutation affecting the glucose porter is affecting glcU, such as within the promoter region of glcU provides for this increased releasing a concentration of glucose assayed as described in example 6, which is at least 50 mM, such as at least 60, 70, 80 or 90 mM. In some embodiment the mutation within the promoter region of glcU is as described herein.

In some embodiments, the present invention is directed to a lactose-positive, sucrose-negative, Streptococcus thermophilus strain carrying a mutation in 2 or 3 genes selected from the group consisting of 1) a gene encoding a protein of the mannose-glucose-specific PTS and a glcK gene, 2) a gene encoding a protein of the mannose-glucose-specific PTS and a ccpA gene, and 3) a gene encoding a protein of the mannose-glucose-specific PTS, a glcK gene and a ccpA gene (as described in the examples of WO2019122365 and WO2019197051).

In some embodiments, the lactose-positive, sucrose-negative Streptococcus thermophilus strain of the invention do not carry any mutation in a gene selected from a gene encoding a protein of the mannose-glucose-specific PTS, a glcK gene and a ccpA gene.

For the avoidance of doubt, the Streptococcus thermophilus species is to be understood as a Streptococcus salivarius subsp. thermophilus strain.

By the expression “lactose-positive”, it is meant a Streptococcus thermophilus strain which is able to grow on lactose as a sole source of carbohydrate source, in particular on a M17 medium supplemented with 3% lactose as described in Test A. In one embodiment, the “lactose-positive” phenotype is assayed by inoculating-into a M17 broth containing 3% lactose—an overnight culture of the S. thermophilus strain to be tested at a rate of 1%, and incubating for 20 hours or alternatively 24 hours at 37° C., and wherein a pH of 5.5 or lower at the end of incubation is indicative of a lactose-positive phenotype.

By the expression “galactose-negative”, it is meant a Streptococcus thermophilus strain which is not able to grow on galactose as a sole source of carbohydrate source, in particular on a M17 medium supplemented with 2% galactose. In a particular embodiment, the “galactose-negative” phenotype is assayed by inoculating-into a M17 broth containing 2% galactose—an overnight culture of the S. thermophilus strain to be tested at 1% and incubating for 20 hours at 37° C., and wherein a pH of 6 or above at the end of incubation is indicative of a galactose-negative phenotype.

By the expression “derivative” in reference to a parental strain (e.g., DSM28255-derivative), it is meant a strain obtained from an original strain (e.g., from the DSM28255-strain) by replacement of one of its genes (such as scrA, glcK, manM, . . . ) by another allele (in particular an allele carrying some specified mutations) of the same gene, or by replacing one gene by a mutated gene. In an embodiment, the derivative is obtained by the replacement of the gene and its promoter of the original strain by another allele of that gene and promoter. In an embodiment, the derivative is obtained by the replacement of all or part of the coding sequence of a gene of the original strain by another allele of that gene or part of it.

For embodiments requiring any of glcK sequencing, transfer of the glcK allele of a suitable strain into the genome of other S. thermophilus strains, screening and selection of Streptococcus thermophilus strains for glucose-excreting properties, identification of relevant mutations in the glcK gene, the identification of and use of further glcK mutations, such as the non-conservative amino acid difference G144S, the identification of and use of specific mutations in the glcK gene, such as the non-conservative amino acid difference, E275K, in the Glck sequence, reference is made to co-pending international patent application with publication number WO2019197051A1.

A derivative of the DSM28255 strain was designed, into which the glcK gene encodes a glucokinase with the glutamic acid (E) at position 275 was replaced by the amino acid lysine (K). This derivative (DGCC12534) was deposited at the DSMZ on Aug. 15, 2017 under accession number DSM32587. The sequence of its GlcK protein is as defined in SEQ ID NO: 22.

### Milk Acidifying Performance

The acidifying properties of S. thermophilus strains may be evaluated by recording the pH over time, during milk fermentation as described in Example 5. The pH is monitored for 24 hours using the CINAC system (Alliance Instruments, France; pH electrode Mettler 405 DPAS SC, Toledo, Spain). The pH is measured and recorded every 5 or 25 minutes. Using the CINAC v2.07 software, the slope between pH 6.0 and pH 5.5 (UpH/minute) [Slope pH6-5.5] is calculated.

Thus, the present invention is directed to sucrose-negative strains of Streptococcus thermophilus. This phenotype of a Streptococcus thermophilus strain is accomplished by either one or more mutations in:
1) one or more mutation in one or more gene of the sucrose regulon; which mutation provides for a significant reduction in the ability to grow on and/or transport sucrose by said strain; and which mutation is in one or more gene of the sucrose regulon, such as the scrA, and/or the scrB gene and/or the scrR gene, optionally with a further mutation in the promoter region regulating the expression of the scrA, scrB and/or scrR genes; and
2) optionally one or more further mutation affecting the glucose porter, such as affecting glcU and/or its expression, which mutation restores or improves glucose consumption of said strain.

### The Sucrose Phosphoenolpyruvate-Sugar Phosphotransferase System (PTS) and the Sucrose Regulon

The term “sucrose phosphoenolpyruvate-sugar phosphotransferase system (PTS)” refers to the sucrose phosphoenolpyruvate-sugar phosphotransferase system (PTS) in its usual meaning of the system used by bacteria for sugar uptake where the source of energy is from phosphoenolpyruvate (PEP). It consists of two non-specific energy-coupling components involved in all the PTSs, i.e. enzyme I (EI) and a heat-stable protein (HPr), and one sucrose-specific multi-domains permease EIIABC^{sucrose} encoded by scrA, that internalizes sucrose as sucrose-6-phosphate. Three other genes are genetically associated to scrA (same genetic locus) on a sucrose regulon; namely scrB (codes for a sucrose-6-phosphate hydrolase), scrR (encodes a GalR-LacI-type transcription regulator) and scrK (encodes a Fructokinase). They are located on 2 operons that are divergently transcribed (scrAK on one side and scrBR on the other side) with the operon operator in between. The term “sucrose regulon” as used herein thus refers to the scrA gene and the genes genetically associated to scrA, namely scrB, scrR and scrK.

In some embodiments the sucrose-negative phenotype is conferred by a mutation in the scrA gene. The present inventors have demonstrated this with one single mutation (scrA₈₂₉) in 3 different genetic backgrounds, which mutation is an insertion of one single base-pair (bp) in position 133 causing a frameshift of the open-reading-frame (ORF) with the consequence of a truncated EIIABC^{sucrose} protein of 45 aa out of 539 aa for the native protein, which effectively is a gene disruption making the truncated protein product inactive. Thus, it may be expected that most frameshifts (insertions or deletions (indels), except insertion or deletion of 3 bp, and multiples of 3 bp) in the gene will have identical effect of gene disruption making the protein product inactive. SNPs (single nucleotide polymorphism), in particular SNPs generating stop codons or that affecting amino acids involved in the catalytic site of the protein, are expected to have similar effects of affecting the enzyme active sites and suppressing the activity.

In some embodiments the sucrose-negative phenotype is conferred by a mutation in the scrB gene. In some embodiments the sucrose-negative phenotype is conferred by a mutation in the scrR gene. In some embodiments the one or more gene of the sucrose regulon involves one or more mutation in one or more of the genes scrA, scrB, and the scrR gene, disrupting the function of one or more of these genes.

Mutations in the Promoter Region Regulating the Expression of the scrA, scrB and/or scrR Genes.

The pscrB region is the operator region driving the expression of the divergently transcribed genes of the sucrose-regulon. This promoter region drives the transcription of scrA and scrK on one side and scrB and scrR on the other side. We have characterized one single mutation referred to as “pscrB₈₂₉”. This specific mutation was being transferred into multiple strains with the same phenotypical consequences, facilitating the strain sucrose-negative. The mutation is a SNP located in a region likely to be the promoter region of scrB (in the area likely to contain the −10 and −35 sequence of the promoter), or in particular a SNP in promoter region of scrAB leading to a transition of T to G, at position-38 of scrB promoter.

In some embodiments the sucrose-negative phenotype is conferred by a mutation in the pscrB region.

Mutations Affecting the Glucose Porter, Such as Affecting glcU and/or its Expression.

Gene glcU encodes a non-PTS transporter (porter) of glucose, also referred to as the “non-PTS glucose permease” or simply the “glucose porter”. It has its own promoter region (pglcU). Some mutations in pglcU allow strains bearing the scrA₈₂₉ and/or pscrB₈₂₉ mutation to recover a growth rate on glucose similar to that of the parental strain. The present inventors have characterized 4 different mutations. One is a SNP, 2 are insertions at the same position but different nucleotide, and the 3rd is an insertion of an IS. All allowed the full recovery of the growth on glucose. The present inventors have characterized mutations in 3 independent genetic backgrounds. All three strains fully recover the ability to grow on glucose. Accordingly, such a mutation of the glucose porter may restore or improve glucose consumption by the strain. As used herein “restores or improves glucose consumption” in relation to a mutation affecting the glucose porter by a strain refers to a strain, which after this mutation either recover from e.g., a mutation in the sucrose regulon to a growth rate similar to or higher than that of the parental strain without such mutation in the sucrose regulon, or alternative which mutation affecting the glucose porter of a strain just improve the strains' ability to grow on glucose. In some embodiments, this term “restores or improves glucose consumption” means that such mutation will give the strain a glucose-positive phenotype and be considered glucose-positive as defined herein.

The present inventors have performed reverse genetics on one pglcU mutant. The pglcU mutation in one of the mutants was reverted to the original wild-type (WT, parental) sequence. This reversion resulted in a glucose slow-growing strain, demonstrating that the pglcU mutation by itself was responsible for the glucose-positive phenotype of the triple mutant (scrA, pscrB, pglcU). In addition, introducing through genetic engineering in a sucrose-negative and glucose slow-growing strain, the pglcU mutation renders the strain glucose-positive.

In one specific embodiment the mutation affecting the glucose porter is the mutation identified as pglcU^{IN_T}, wherein the insertion of a T nucleotide in the poly-T at position corresponding to −75 of the glcU promoter restores/improves glucose uptake and consumption.

In another specific embodiment the mutation affecting the glucose porter is the mutation identified as pglcU^{IN_C}, wherein the insertion of a C nucleotide in the poly-T region at position corresponding to −75 of the glcU promoter restores/improves glucose uptake and consumption.

In another specific embodiment the mutation affecting the glucose porter is the mutation identified as pglcU^{SNP_C}, wherein a mutation of a T to a C nucleotide in the poly-T region at position corresponding to −81 of the glcU promoter restores/improves glucose uptake and consumption.

In another specific embodiment the mutation affecting the glucose porter is the mutation identified as pglcU^{IS}, wherein an insertion of a sequence at a position corresponding to between nucleotide position 110 and 111 relative to the sequence of strain ST1-ABU4 identified herein or of ST1 identified by SEQ ID NO: 216 of the glcU promoter, which insertion restores and/or improves glucose uptake and consumption.

It is expected that a scrA₈₂₉ mutation may have pleiotropic effects on the expression of other genes involved in carbohydrate utilization. This effect is likely to take place at the transcription level (as suggested by the location of the mutation in pglcU, and by the common knowledge on the regulation of the catabolism of carbohydrate). Thus, a scrA₈₂₉ mutation is likely to down regulate glcU expression, and mutation in pglcU, possibly reverts this down-regulation, possibly making the expression constitutive.

Accordingly, in some embodiments the lactose-positive, sucrose-negative, Streptococcus thermophilus strain according to the present invention is glucose-positive, or glucose slow-growing. A glucose-positive strain may thus be propagated and produced using glucose rather than lactose.

As used herein a “glucose-positive” strain refers to a Streptococcus thermophilus strain able to grow on glucose as a sole source of carbohydrate, such as wherein the ratio of the speed of growth on glucose as a sole source of carbohydrate (μ_{glucose}) (as defined and measured accordingly to Test B described herein at the timepoint corresponding to the maximal growth rate of the same strain has on lactose (μmax_{lactose})) over μmax_{lactose} (μ_{glucose}/μmax_{lactose} ratio) is equal to or higher than 0.25, such as higher than 0.3, such as 0.35, 0.4, 0.5, 0.6, 0.7, 0.8, or 0.9 indicating that strains grow well on glucose. Alternatively, a Streptococcus thermophilus strain is considered “glucose-positive” when the μ_{glucose} value, defined and measured accordingly to Test B described herein, is equal to or higher than 0.20, such as higher than 0.25, 0.3, 0.4, 0.5, 0.6, 0.7, or 0.8.

As used herein a “glucose slow-growing” strain refers to a Streptococcus thermophilus strain wherein the ability to grow on glucose as a sole source of carbohydrate is not completely abolished but significantly slower than for a glucose-positive strain, such as wherein the ratio of the speed of growth on glucose as a sole source of carbohydrate (μ_{glucose}) (as defined and measured accordingly to Test B described herein at the timepoint corresponding to the maximal growth rate of the same strain has on lactose (μmax_{lactose})) over μmax_{lactose} (μ_{glucose}/μmax_{lactose} ratio) is lower than 0.25, such as lower than 0.24, 0.23, 0.22, 0.21, 0.20, 0.19, 0.18, or 0.17, but still higher than 0.05 indicating that strains has an ability but slow growth on glucose. Alternatively, a Streptococcus thermophilus strain is considered “glucose-slow growing” when the μ_{glucose} value, defined and measured accordingly to Test B described herein, is higher than 0.01, but lower than 0.2, such as lower than 0.18, 0.17, 0.16, 0.15, 0.14, 0.13, or 0.12.

In dairy application, it is expected for the strain of the invention to not consume sucrose, or at least to consume a significant lower amount of sucrose than a parent strain. In order to evaluate the sucrose-negative phenotype the strains of the present invention are tested for the ability to grow on a medium containing sucrose as a sole source of carbohydrate. For example, through the kinetic measurement of OD of a culture of the strain in a synthetic medium (M17) containing sucrose (30 g/L) as a sole source of carbohydrate at appropriate temperature (37° C.). Alternatively, enzymatic means may be considered. For example, by measuring the ability of resting cells of a scrA mutant to transport sucrose; or the ability of an intracellular extract of a scrB mutant to hydrolyze phosphorylated sucrose (sucrose-6-phosphate). In a preferred assay the sucrose-negative phenotype of the strains of the present invention are tested as described in Test A as defined herein.

In some embodiments, the strains according to the present invention comprises a mutation in the gene encoding a protein of the mannose-glucose-specific PTS that either alone or in combination with mutations of other genes may reduce or abolish the import of glucose from the medium into the bacteria. In some embodiments, a mutated glcK gene encodes a glucokinase, the glucokinase activity of which in said strain is significantly reduced but not null. In some embodiments, said lactose-positive, sucrose-negative, Streptococcus thermophilus strain carries a mutation in a gene encoding a protein of the mannose-glucose-specific PTS reducing or abolishing the import of glucose from the medium into the bacteria and carrying a mutation in the glcK gene encodes a glucokinase, such that the glucokinase activity of which in said strain is significantly reduced but not null in said strain. In any of these embodiments, the gene encoding a protein of the mannose-glucose-specific PTS may be selected from the group consisting of the manL gene, the manM gene and the manN gene.

In some embodiments, a mutation in the gene encoding a protein of the mannose-glucose-specific PTS either alone or in combination with mutations of other genes reduces or abolishes the import of glucose from the medium into the bacteria. In some embodiments, the mutation in the ccpA gene leads to a lactose-positive, sucrose-negative, Streptococcus thermophilus strain exhibiting a ratio of the beta-galactosidase activity of said strain as assayed by the Beta-galactosidase Activity Assay described herein over the glucokinase activity of said strain as assayed by The Glucokinase Activity Assay which is at least 4·10⁻⁶, at least 5·10⁻⁶, at least 6·10⁻⁶, at least 7·10⁻⁶ or at least 8·10⁻⁶. In some embodiments, said lactose-positive, sucrose-negative Streptococcus thermophilus strain carries a mutation in a gene encoding a protein of the mannose-glucose-specific PTS reducing or abolishing the import of glucose from the medium into the bacteria and carrying a mutation in the ccpA gene such that the ratio of the beta-galactosidase activity of said strain as assayed by the Beta-galactosidase Activity Assay described herein over the glucokinase activity of said strain as assayed by The Glucokinase Activity Assay which is at least 4·10⁻⁶.

In some embodiments, the mutation in the gene encoding a protein of the mannose-glucose-specific PTS either alone or in combination with mutations of other genes reduces or abolishes the import of glucose from the medium into the bacteria. In some embodiments, a mutated glcK gene encodes a glucokinase, the glucokinase activity of which in said strain is significantly reduced but not null in said strain. In some embodiments, said lactose-positive, sucrose-negative, Streptococcus thermophilus strain carrying a mutation in the glcK gene also carries a mutation in the ccpA gene.

The following parts describe respectively mutations of the glcK gene, mutations of the gene encoding a protein of the mannose-glucose-specific PTS (such as mutations of the manL, manM and manN genes) and mutations of the ccpA gene.

Though these mutations are disclosed herein separately (for sake of clarity), any embodiment of one part can be combined with any embodiment of another part or with any embodiment of the two other parts, to design a lactose-positive, sucrose-negative, Streptococcus thermophilus strain as defined herein.

For the avoidance of doubt, in some embodiments the present invention is directed to a lactose-positive, sucrose-negative, Streptococcus thermophilus strain as defined herein, wherein said strain carries:
1) a mutation in at least one, in particular one, gene encoding a protein of the mannose-glucose-specific PTS and a mutation in its glcK gene; or
2) a mutation in at least one, in particular one, gene encoding a protein of the mannose-glucose-specific PTS and a mutation in its ccpA gene; or
3) a mutation in at least one, in particular one, gene encoding a protein of the mannose-glucose-specific PTS, a mutation in its glcK gene and a mutation in its ccpA gene.
   Mutations of the glcK Gene

This part describes mutations of the glcK gene which can be used in combination with mutations in one or more gene of the sucrose regulon, mutations affecting the glucose porter, mutations of a gene encoding a protein of the mannose-glucose-specific PTS, and/or in combination with a mutation of a gene encoding a protein of the mannose-glucose-specific PTS as defined herein and a mutation of the ccpA gene as defined herein, in the context of a lactose-positive, sucrose-negative, Streptococcus thermophilus strain of the invention.

In some embodiments, a mutated glcK gene of a strain of the invention encodes a glucokinase, the glucokinase activity of which in said strain is significantly reduced but not null. Indeed, the inventors have put in evidence that some mutated alleles of the glcK gene codes for a glucokinase (Glck), the glucokinase activity of which is significantly reduced but not null, when said mutated glcK gene is present in a lactose-positive Streptococcus thermophilus strain.

The expression “glcK gene encoding a glucokinase” means any DNA sequence of a Streptococcus thermophilus strain encoding the glucokinase enzyme which catalyses the conversion of glucose and ATP to glucose-6-phosphate (G6P) and ADP. Non-limitative examples of Streptococcus thermophilus glucokinase sequences are disclosed as SEQ ID Nos: 2, 4, 6, 8, 10, 12, 14, 16, 18 and 20.

In some embodiments within the present invention, the glucokinase activity in a Streptococcus thermophilus strain is significantly reduced but not null as a consequence of a mutation in its glcK gene. In other words, the allele of the glcK gene carried by said strain is such that the glucokinase activity in said strain is significantly reduced but not null.

The expression “glucokinase activity in said strain is significantly reduced but not null” refers to a strain the glucokinase activity of which is both:
significantly reduced in said strain, in particular as compared to the glucokinase activity in a strain carrying a non-mutated glcK gene; and
not null, i.e., that an activity is detectable by the Glucokinase Activity Assay as defined herein.

According to embodiments of the invention, the feature “glucokinase activity in said strain is significantly reduced but not null” can be determined by methods well known in the art. Thus, methods for measuring the glucokinase activity in a Streptococcus thermophilus strain are known and include enzyme assays with commercially available reactants. Reference is made herein to the paragraph 2.4 of Pool et al. (2006. Metabolic Engineering 8 (5); 456-464) (incorporated herein by reference). In a particular embodiment, the glucokinase activity in a Streptococcus thermophilus strain of the invention is assayed by the Glucokinase Activity Assay [i.e. the Glucokinase Activity Assay is carried out using the Streptococcus thermophilus strain of the invention].

### Glucokinase Activity Assay:

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) at a 600 nm optical density (OD600) of 0.8+/−0.2, washed in 5 ml cold GLCK buffer (5 mM MgCl2, 10 mM K₂HPO₄/KH₂PO₄ [pH 7.2]), and resuspended in 500 μl cold GLCK buffer. EDTA-free protease inhibitors “cOmplete™” (Roche, supplier reference 04693132001) is added in GLCK buffer as described by the provider. Cells are disrupted by the addition of 100 mg glass beads (150-212 μm, Sigma G1145) to 200 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The glucokinase activity in the cell extracts is determined spectrophotometrically by a glucose-6-phosphate dehydrogenase (G-6PDH, EC1.1.1.49):NADPH-coupled assay (Porter et al., 1982), essentially as described by Pool et al. (2006). Each sample (5, 10 and 20 μL) is added to assay buffer (10 mM K₂HPO₄/KH₂PO₄ [pH 7.2], 5 mM MgCl2, 1 mM ATP, 20 mM glucose, 1 mM NADP, 1 U G-6PDH) in a 250 μL final volume, and the mixture was left for 5 min at 30° C. The optical density at 340 nm is measured for 5 minutes by using a Synergy HT multi-detection microplate reader (BIO-TEK). One unit of glucokinase corresponds to the amount of enzyme that catalyzes the phosphorylation of 1 μmole of D-glucose to D-Glucose 6-phosphate per minute under the assay conditions. Glucokinase activity is calculated as follows:

Glucokinase activity (U/g of total protein extract)=dOD×V/[dt×l×ε×Qprot], wherein:
dOD is the variation of optical density (OD) at 340 nm
V is the volume of the reaction (herein 250 μL)
dt=measurement time (in minutes)
l=optical path length (herein 0.73 cm)
ε=molar attenuation coefficient of NADPH; H⁺ (herein 6220 cm2/μmol)
Qprot=quantity of protein in the cuvette (in g)
   Measurements are triplicated for each sample, and the glucokinase specific activity values given herein under the Glucokinase Activity Assay are the mean of three independent experiments.

In a first particular embodiment of the feature “glucokinase activity in said strain is significantly reduced but not null”, the glucokinase activity in the Streptococcus thermophilus strain of the invention is between 200 and 1500 U/g of total protein extract, as assayed by the Glucokinase Activity Assay. In a particular embodiment, the glucokinase activity in the Streptococcus thermophilus strain of the invention is between 300 and 1200 U/g of total protein extract, as assayed by the Glucokinase Activity Assay. In a particular embodiment, the glucokinase activity in the Streptococcus thermophilus strain of the invention is between 400 and 1000 U/g of total protein extract, as assayed by the Glucokinase Activity Assay. In a particular embodiment, the glucokinase activity in the Streptococcus thermophilus strain of the invention is between a minimal value selected from the group consisting of 200, 300 and 400 U/g of total protein extract and a maximal value selected from the group consisting of 1000, 1200 and 1500 U/g of total protein extract, as assayed by the Glucokinase Activity Assay. It is noteworthy that, as mentioned in the Glucokinase Activity Assay, the glucokinase activity values disclosed herein are the mean of three independent experiments (triplicates).

In a second particular embodiment of the feature “glucokinase activity in said strain is significantly reduced but not null”, the glucokinase activity in the Streptococcus thermophilus strain of the invention is between 5 and 60% the activity of the glucokinase activity of the DGCC7710 strain deposited at the DSMZ under accession number DSM28255 on Jan. 14, 2014. By “glucokinase activity of the DSM28255 strain”, it is meant the activity of the DSM28255 strain glucokinase (i.e., with SEQ ID NO: 2) as assayed by the Glucokinase Activity Assay in the DSM28255 strain [i.e., the Glucokinase Activity Assay is carried out using the DSM28255 strain]. The percentage value is calculated based on the glucokinase activity in the strain of the invention and the glucokinase activity of the DSM28255 strain, both assayed by the Glucokinase Activity Assay. In a particular embodiment, the glucokinase activity in the Streptococcus thermophilus strain of the invention is between 10 and 50% the glucokinase activity of the DSM28255 strain. In a particular embodiment, the glucokinase activity in the Streptococcus thermophilus strain of the invention is between 15 and 40% the glucokinase activity of the strain DSM28255. In a particular embodiment, the glucokinase activity of the Streptococcus thermophilus strain of the invention is between a minimal percentage selected from the group consisting of 5, 10 and 15% the glucokinase activity of the DSM28255 strain and a maximal percentage selected from the group consisting of 40, 50 and 60% the glucokinase activity of the DSM28255 strain. In a particular embodiment and whatever the range of percentages, the activity of the glucokinase activity is assayed by the Glucokinase Activity Assay as described herein. It is noteworthy that the percentage values disclosed herein are calculated based on glucokinase activity values which are the mean of three independent experiments (triplicates) as assayed by the Glucokinase Activity Assay.

In the first and second particular embodiments, the following strains can be used as controls in the Glucokinase Activity Assay:
as a positive control (i.e., a Streptococcus thermophilus strain, which is representative of strains carrying a non-mutated glcK gene): strain DGCC7710 deposited at the DSMZ under accession number DSM28255 on Jan. 14, 2014.

The feature “glucokinase activity in said strain is significantly reduced but not null” can also be characterized by the maximum forward velocity of the glucokinase (herein called Vmax, and defined as the velocity of the Glucose+ATP conversion to G6P+ADP) or by the inverse of the affinity of the glucokinase (called Km) for one or two of its substrates, i.e., glucose and ATP. In some embodiments, the feature “glucokinase activity in said strain is significantly reduced but not null” for the strain of the invention is further characterized by the maximum forward velocity (Vmax) of its glucokinase in said strain.

Therefore, in combination with the first or second particular embodiment of the feature “glucokinase activity in said strain is significantly reduced but not null” defined herein, the maximum forward velocity (Vmax) of the glucokinase in the lactose-positive, sucrose-negative, Streptococcus thermophilus strain of the invention is significantly reduced but not null. The feature “glucokinase Vmax in said strain is significantly reduced but not null” can be defined by one or two of these parameters:
the Vmax is between 200 and 1500 U/g total protein extract, as assayed by The Glucokinase Vmax Assay.
the Vmax is between 5 and 60% the Vmax of the glucokinase of the DSM28255 strain deposited at the DSMZ under accession number DSM28255 on Jan. 14, 2014, when both assayed by The Glucokinase Vmax Assay.

In a particular embodiment, a mutated glcK gene of a lactose-positive, sucrose-negative, Streptococcus thermophilus strain of the invention encodes a glucokinase, wherein the glucokinase activity in said strain is significantly reduced but not null (as defined herein), and wherein the maximum forward velocity (Vmax) of its glucokinase in said strain is significantly reduced but not null and defined by one or two of these parameters:
the Vmax is between 200 and 1500 U/g total protein extract, as assayed by The Glucokinase Vmax Assay.
the Vmax is between 5 and 60% the Vmax of the glucokinase of the DSM28255 strain deposited at the DSMZ under accession number DSM28255 on Jan. 14, 2014, when both assayed by The Glucokinase Vmax Assay.

The glucokinase maximum forward velocity (Vmax) in a Streptococcus thermophilus of the invention is assayed by The Glucokinase Vmax Assay [carried out using the Streptococcus thermophilus strain of the invention].

### The Glucokinase Vmax Assay:

The maximal forward velocity (Vmax) is determined by using various concentrations of glucose (0, 5, 10, 15, 20 mM) on crude extract prepared as described in the Glucokinase Activity Assay. Measurements are triplicated for each sample, and the Vmax values given under The Glucokinase Vmax Assay are the mean of three independent experiments. The linear regression representing the inverse of the specific velocity in function of the inverse of the glucose concentration gives the inverse of the maximal forward velocity at the intersection with the Y-axis of the graphic.

In a particular embodiment of the maximum forward velocity of the glucokinase in the Streptococcus thermophilus strain of the invention, the Vmax is between 200 and 1500 U/g total protein extract, as assayed by The Glucokinase Vmax Assay. In a particular embodiment, the Vmax is between 300 and 1200 U/g total protein extract, as assayed by The Glucokinase Vmax Assay. In a particular embodiment, the Vmax is between 400 and 1000 U/g total protein extract. In a particular embodiment, the Vmax of the glucokinase in the Streptococcus thermophilus strain of the invention is between a minimal value selected from the group consisting of 200, 300 and 400 U/g of total protein extract and a maximal value selected from the group consisting of 1000, 1200 and 1500 U/g of total protein extract, as assayed by The Glucokinase Vmax Assay.

In a particular embodiment of the maximum forward velocity of the glucokinase in the Streptococcus thermophilus strain of the invention, the Vmax is between 5 and 60% the Vmax of the glucokinase of the DSM28255 strain. By “Vmax of the glucokinase of the DSM28255 strain”, it is meant the Vmax of the DSM28255 strain glucokinase (i.e., with SEQ ID NO: 2) as assayed by The Glucokinase Vmax Assay in the DSM28255 strain [i.e., the The Glucokinase Vmax Assay is carried out using the DSM28255 strain]. The percentage value is calculated based on the Vmax of the glucokinase in the strain of the invention and the Vmax of the DSM28255 strain, both assayed by The Glucokinase Vmax Assay. In a particular embodiment, the glucokinase Vmax in the Streptococcus thermophilus strain of the invention is between 10 and 50% the Vmax of the glucokinase of the DSM28255 strain, when both assayed by The Glucokinase Vmax Assay. In a particular embodiment, the glucokinase Vmax in the Streptococcus thermophilus strain of the invention is between 15 and 40% the Vmax of the glucokinase of the DSM28255 strain. In a particular embodiment, the Vmax of the glucokinase in the Streptococcus thermophilus strain of the invention is between a minimal percentage selected from the group consisting of 5, 10 and 15% the Vmax of the glucokinase activity of the DSM28255 strain and a maximal percentage selected from the group consisting of 40, 50 and 60% the Vmax of the glucokinase activity of the DSM28255 strain.

The lactose-positive, sucrose positive Streptococcus thermophilus strain of the invention may carry a mutation in the glcK gene encoding a glucokinase, the glucokinase activity of which in said strain is significantly reduced but not null as defined herein and optionally wherein the maximum forward velocity of the glucokinase in said strain is significantly reduced but not null as defined herein.

By “mutation in the glcK gene” within the present invention, it is meant any nucleotide variation within the glcK gene, wherein said variation at the nucleotide level leads to a glucokinase activity in a strain carrying this mutated glcK gene (as the sole glcK gene) which is significantly reduced but not null as defined herein and optionally leads to a maximum forward velocity of the glucokinase in said strain which is significantly reduced but not null as defined herein. In a particular embodiment, by “mutation in the glcK gene” within the present invention, it is meant any nucleotide variation within the open reading frame of the glcK gene, wherein said variation at the nucleotide level leads to a glucokinase activity in a strain carrying this mutated glcK gene (as the sole glcK gene) which is significantly reduced but not null as defined herein and optionally leads to a maximum forward velocity of the glucokinase in said strain which is significantly reduced but not null as defined herein.

Thus, though two Streptococcus thermophilus strains may differ by the sequence of their respective glcK gene, this does not necessarily mean that one of these two glcK genes is mutated in the sense of the invention. Indeed, are not considered as mutations within the present invention:
variations at the nucleotide level which do not lead to any change at the protein level (silent variation) and which do not impact the translation of the glcK RNA; and
variations at the nucleotide level which do lead to a change at the protein level, but wherein this change does not impact the glucokinase activity of the resulting Glck protein and optionally the maximum forward velocity of the resulting Glck protein, as defined herein. Indeed, such variations can be observed at the level of the glcK gene of the Streptococcus thermophilus of the invention without impacting the scope of protection.

Non-limitative examples of glcK genes which are not considered as mutated in the sense of the invention are:
the polynucleotide encoding the glucokinase as defined in SEQ ID NO:2, in particular the polynucleotide as defined in SEQ ID NO: 1; this GlcK type is the one of DGCC7710 strain deposited at the DSMZ under accession number DSM28255 on Jan. 14, 2014;
the polynucleotide encoding the glucokinase as defined in SEQ ID NO:4, in particular the polynucleotide as defined in SEQ ID NO:3;
the polynucleotide encoding the glucokinase as defined in SEQ ID NO:6, in particular the polynucleotide as defined in SEQ ID NO:5;
the polynucleotide encoding the glucokinase as defined in SEQ ID NO:8, in particular the polynucleotide as defined in SEQ ID NO:7;
the polynucleotide encoding the glucokinase as defined in SEQ ID NO: 10, in particular the polynucleotide as defined in SEQ ID NO:9;
the polynucleotide encoding the glucokinase as defined in SEQ ID NO: 12, in particular the polynucleotide as defined in SEQ ID NO:11;
the polynucleotide encoding the glucokinase as defined in SEQ ID NO: 14, in particular the polynucleotide as defined in SEQ ID NO: 13;
the polynucleotide encoding the glucokinase as defined in SEQ ID NO: 16, in particular the polynucleotide as defined in SEQ ID NO: 15;
the polynucleotide encoding the glucokinase as defined in SEQ ID NO: 18, in particular the polynucleotide as defined in SEQ ID NO: 17;
the polynucleotide encoding the glucokinase as defined in SEQ ID NO:20, in particular the polynucleotide as defined in SEQ ID NO: 19.

Moreover, some nucleotide mutations within the glcK gene are not considered suitable to the invention, because they lead to a glucokinase, the activity of which is null or is under the minimal value defined herein, as assayed by the Glucokinase Activity Assay. In some embodiments, the Streptococcus thermophilus of the invention does not carry a mutation selected from the group consisting of a mutation leading to the knock-out of the glcK gene and large deletions within the glcK gene.

In some embodiments, a lactose-positive, sucrose-negative Streptococcus thermophilus strain of the invention carries a mutation in the open reading frame of the glcK gene leading to the substitution of an amino acid in the Glck protein, the glucokinase activity of which in said strain carrying a mutated glcK gene is significantly reduced but not null (as defined herein) and optionally wherein the maximum forward velocity of the glucokinase in said strain is significantly reduced but not null as defined herein. In a particular embodiment, a lactose-positive, sucrose-negative Streptococcus thermophilus strain of the invention carries a mutation in the glcK gene leading to the substitution of an amino acid in the Glck protein, the glucokinase activity of which in said strain carrying a mutated glcK gene is significantly reduced but not null (as defined herein) and optionally wherein the maximum forward velocity of the glucokinase in said strain is significantly reduced but not null as defined herein. In a particular embodiment, the Streptococcus thermophilus strain of the invention carries a mutation in the glcK gene such that the Glck protein is 322-amino acids in length and wherein the glucokinase activity in said strain is significantly reduced but not null as defined herein and optionally wherein the maximum forward velocity of the glucokinase in said strain is significantly reduced but not null as defined herein.

As discussed above, some DNA modifications can be observed at the level of the glcK gene of the Streptococcus thermophilus of the invention which do not impact the glucokinase activity of the strain. Based on the Glucokinase Activity Assay defined herein together with the control strains defined herein, the person skilled in the art would know how to identify 1) a glcK gene encoding a glucokinase, the glucokinase activity of which in a strain carrying this glcK gene is significantly reduced but not null (as defined herein) and optionally wherein the maximum forward velocity of the glucokinase in a strain carrying this mutated glcK gene is significantly reduced but not null (as defined herein), 2) a glcK gene bearing a modification having no impact on the glucokinase activity in a strain carrying this modification or 3) a glcK gene encoding a glucokinase, the glucokinase activity of which in a strain carrying this glcK gene is null (as defined herein).

The present inventors have identified two positions within the glucokinase, for which the amino acid nature has been shown to impact the activity of the glucokinase, such that the glucokinase activity is significantly reduced but not null as defined herein and to impact the Vmax of the glucokinase such that the Vmax is significantly reduced but not null as defined herein: position 144 and position 275 of the glucokinase (i.e., codon 144 and 275 of the glcK gene). It is noteworthy that based on the Glucokinase Activity Assay and the Glucokinase Vmax Assay defined herein together with the control strains, the person skilled in the art would know how to identify other positions and appropriate amino acids within the glucokinase, to obtain a glucokinase activity significantly reduced but not null (as defined herein) and optionally a maximum forward velocity which is significantly reduced but not null, and thus the corresponding glcK gene.

In some embodiments, the amino acid at position 275 of the glucokinase (encoded by the glcK gene of the Streptococcus thermophilus strain of the invention) is not a glutamic acid (i.e., is any amino acid except a glutamic acid); thus, in some embodiments, the codon 275 of the glcK gene carried by the Streptococcus thermophilus strain of the invention is neither GAA nor GAG. In a particular embodiment, the amino acid at position 275 of the glucokinase is not an acidic amino acid (i.e., is any amino acid except an acidic amino acid); thus, in some embodiments, the codon 275 of the glcK gene carried by the Streptococcus thermophilus strain of the invention is a codon encoding a non-acidic amino acid. In a particular embodiment, the amino acid at position 275 of the glucokinase is selected from the group consisting of lysine and any of its conservative amino acids; thus, in some embodiments, the codon 275 of the glcK gene carried by the Streptococcus thermophilus strain of the invention is a codon encoding an amino acid selected from the group consisting of a lysine and any of its conservative amino acids. In a particular embodiment, the amino acid at position 275 of the glucokinase is a lysine; thus, in some embodiments, the codon 275 of the glcK gene carried by the Streptococcus thermophilus strain of the invention is either AAA or AAG. In a particular embodiment, the nucleotides 823-825 of the glcK gene carried by the Streptococcus thermophilus strain of the invention are AAA or AAG.

In a particular embodiment, the sequence of the Glck protein of a lactose-positive, sucrose-negative Streptococcus thermophilus strain of the invention is selected from the group consisting of:
a) a sequence as defined in SEQ ID NO: 25, wherein the amino acid at position 275 is any amino acid except a glutamic acid, in particular is any amino acid except an acidic amino acid, in particular is a lysine; and
b) a Glck variant sequence having at least 90% similarity or identity with SEQ ID NO: 25, wherein the amino acid of the glucokinase corresponding to position 275 of SEQ ID NO:25 (or the amino acid at position 275 of the glucokinase) is any amino acid except a glutamic acid, in particular is any amino acid except an acidic amino acid, in particular is a lysine. In a particular embodiment, the GlcK variant sequence is 322-amino acids in length.

In another embodiment, the amino acid at position 144 of the glucokinase (encoded by the glcK gene of the Streptococcus thermophilus strain of the invention) is not a glycine (i.e., is any amino acid except a glycine); thus, in some embodiments, the codon 144 of the glcK gene carried by the Streptococcus thermophilus strain of the invention is not GGT, GGC, GGA or GGG. In a particular embodiment, the amino acid at position 144 of the glucokinase is not an aliphatic amino acid (i.e., is any amino acid except an aliphatic amino acid). In a particular embodiment, the amino acid at position 144 of the glucokinase is selected from the group consisting of serine and any of its conservative amino acids; thus, in some embodiments, the codon 144 of the glcK gene carried by the Streptococcus thermophilus strain of the invention is a codon encoding an amino acid selected from the group consisting of a serine and any of its conservative amino acids. In a particular embodiment, the amino acid at position 144 of the glucokinase is a serine; thus, in some embodiments, the codon 144 of the glcK gene carried by the Streptococcus thermophilus strain of the invention is AGT, AGC, TCT, TCC, TCA or TCG. In a particular embodiment, the nucleotides 430-432 of the glcK gene carried by the Streptococcus thermophilus strain of the invention are AGT, AGC, TCT, TCC, TCA or TCG.

In a particular embodiment, the sequence of the GlcK protein of a lactose-positive, sucrose-negative Streptococcus thermophilus strain of the invention is selected from the group consisting of:
a) a sequence as defined in SEQ ID NO:46, wherein the amino acid at position 144 is any amino acid except a glycine, in particular is any amino acid except an aliphatic amino acid, in particular is a serine; and
b) a Glck variant sequence having at least 90% similarity or identity with SEQ ID NO: 46, wherein the amino acid of the glucokinase corresponding to position 144 of SEQ ID NO:46 (or the amino acid at position 144 of the glucokinase) is any amino acid except a glycine, in particular is any amino acid except an aliphatic amino acid, in particular is a serine. In a particular embodiment, the Glck variant sequence is 322-amino acids in length.

For the definition of the Glck variant having at least 90% similarity or identity with SEQ ID NO: 25, the similarity or identity is calculated herein over the whole length of the 2 sequences after optimal alignment [i.e., number of similar or identical amino acid residues in the aligned parts(s) of the sequences]; the position 275 as defined in SEQ ID NO:25 is not considered for the calculation of the similarity or of the identity. In a particular embodiment, the Glck variant sequence has at least 91, 92, 93, 94, 95, 96, 97, 98 or 99% similarity or identity with SEQ ID NO:25, wherein the amino acid corresponding to position 275 of SEQ ID NO: 25 (or the amino acid at position 275 of the glucokinase) is any amino acid except a glutamic acid, in particular is any amino acid except an acidic amino acid, in particular is a lysine. In some embodiments, the Glck variant sequence has at least 95% similarity or identity with SEQ ID NO: 25, wherein the amino acid corresponding to position 275 of SEQ ID NO:25 (or the amino acid at position 275 of the glucokinase) is any amino acid except a glutamic acid, in particular is any amino acid except an acidic amino acid, in particular is a lysine. In some embodiments, the Glck variant sequence has at least 97% similarity or identity with SEQ ID NO: 25, wherein the amino acid corresponding to position 275 of SEQ ID NO: 25 (or the amino acid at position 275 of the glucokinase) is any amino acid except a glutamic acid, in particular is any amino acid except an acidic amino acid, in particular is a lysine.

In a particular embodiment, the GlcK variant sequence differs from SEQ ID NO:25 by from 1 to 30 amino acid substitutions wherein the amino acid at position 275 of said Glck variant is any amino acid except a glutamic acid, in particular is any amino acid except an acidic amino acid, in particular is a lysine (the position 275 is not considered for the calculation of the number of substitution(s)). In a particular embodiment, the Glck variant sequence differs from SEQ ID NO: 25 by from 1 to 20 amino acid substitutions, wherein the amino acid at position 275 of said GlcK variant is any amino acid except a glutamic acid, in particular is any amino acid except an acidic amino acid, in particular is a lysine. In a particular embodiment, the Glck variant sequence differs from SEQ ID NO: 25 by from 1 to 15 amino acid substitutions wherein the amino acid at position 275 of said GlcK variant is any amino acid except a glutamic acid, in particular is any amino acid except an acidic amino acid, in particular is a lysine. In a particular embodiment, the Glck variant sequence differs from SEQ ID NO: 25 by from 1 to 10 amino acid substitutions wherein the amino acid at position 275 of said Glck variant is any amino acid except a glutamic acid, in particular is any amino acid except an acidic amino acid, in particular is a lysine. In a particular embodiment, the Glck variant sequence differs from SEQ ID NO: 25 by 1, 2, 3, 4, 5, 6, 7, 8, 9, 10, 11 or 12 amino acid substitutions, wherein the amino acid at position 275 of said GlcK variant is any amino acid except a glutamic acid, in particular is any amino acid except an acidic amino acid, in particular is a lysine.

For the definition of the Glck variant having at least 90% similarity or identity with SEQ ID NO: 46, the similarity or identity is calculated herein over the whole length of the 2 sequences after optimal alignment [i.e., number of similar or identical amino acid residues in the aligned parts(s) of the sequences]; the position 144 as defined in SEQ ID NO:46 is not considered for the calculation of the similarity or of the identity. In a particular embodiment, the Glck variant sequence has at least 91, 92, 93, 94, 95, 96, 97, 98 or 99% similarity or identity with SEQ ID NO:46, wherein the amino acid corresponding to position 144 of SEQ ID NO: 46 (or the amino acid at position 144 of the glucokinase) is any amino acid except a glycine, in particular is any amino acid except an aliphatic amino acid, in particular is a serine. In some embodiments, the GlcK variant sequence has at least 95% similarity or identity with SEQ ID NO:46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine. In some embodiments, the Glck variant sequence has at least 97% similarity or identity with SEQ ID NO: 46, wherein the amino acid corresponding to position 144 of SEQ ID NO:46 (or the amino acid at position 144 of the glucokinase) is any amino acid except a glycine, in particular is any amino acid except an aliphatic amino acid, in particular is a serine.

In a particular embodiment, the GlcK variant sequence differs from SEQ ID NO:46 by from 1 to 30 amino acid substitutions wherein the amino acid at position 144 of said Glck variant is any amino acid except a glycine, in particular is any amino acid except an aliphatic amino acid, in particular is a serine (the position 144 is not considered for the calculation of the number of substitution(s)). In a particular embodiment, the Glck variant sequence differs from SEQ ID NO:46 by from 1 to 20 amino acid substitutions, wherein the amino acid at position 144 of said GlcK variant is any amino acid except a glycine, in particular is any amino acid except an aliphatic amino acid, in particular is a serine. In a particular embodiment, the Glck variant sequence differs from SEQ ID NO: 46 by from 1 to 15 amino acid substitutions wherein the amino acid at position 144 of said GlcK variant is any amino acid except a glycine, in particular is any amino acid except an aliphatic amino acid, in particular is a serine. In a particular embodiment, the Glck variant sequence differs from SEQ ID NO: 46 by from 1 to 10 amino acid substitutions wherein the amino acid at position 144 of said Glck variant is any amino acid except a glycine, in particular is any amino acid except an aliphatic amino acid, in particular is a serine. In a particular embodiment, the GlcK variant sequence differs from SEQ ID NO: 46 by 1, 2, 3, 4, 5, 6, 7, 8, 9, 10, 11 or 12 amino acid substitutions, wherein the amino acid at position 144 of said GlcK variant is any amino acid except a glycine, in particular is any amino acid except an aliphatic amino acid, in particular is a serine.

In some embodiments, the sequence of the Glck protein of a lactose-positive, sucrose-negative Streptococcus thermophilus strain of the invention is selected from the group consisting of SEQ ID NOs: 25, 26, 27, 28, 29, 30, 31, 32, 33 and 34, wherein the amino acid at position 275 of said variant is any amino acid except a glutamic acid, in particular is any amino acid except an acidic amino acid, in particular is a lysine; thus, in some embodiments, the glcK gene carried by the Streptococcus thermophilus strain of the invention encodes a Glck protein, the sequence of which is selected from the group consisting of SEQ ID NOs: 25, 26, 27, 28, 29, 30, 31, 32, 33 and 34, wherein the amino acid at position 275 of the glucokinase is not a glutamic acid, in particular is not an acidic amino acid, in particular is a lysine respectively.

In a particular embodiment, either as the SEQ ID NO: 25 or any Glck variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is not a glutamic acid; thus, in some embodiments, the codon 275 of the glcK gene carried by the Streptococcus thermophilus strain of the invention is neither GAA nor GAG; thus, in some embodiments, the glcK gene carried by the Streptococcus thermophilus strain of the invention encodes a GlcK protein, the sequence of which is selected from the group consisting of SEQ ID NO: 25 and any GlcK variant sequence having at least 90% similarity or identity with SEQ ID NO: 25 as defined herein (in particular SEQ ID NO: 26, 27, 28, 29, 30, 31, 32, 33 or 34), wherein the amino acid of the glucokinase corresponding to position 275 of SEQ ID NO: 25 (or the amino acid at position 275 of the glucokinase) is not a glutamic acid.

In a particular embodiment, either as the SEQ ID NO:25 or any Glck variant sequence having at least 90% similarity or identity with SEQ ID NO: 25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is not an acidic amino acid; thus, in some embodiments, the codon 275 of the glcK gene carried by the Streptococcus thermophilus strain of the invention is a codon which does not encode an acidic amino acid; thus, in some embodiments, the glcK gene carried by the Streptococcus thermophilus strain of the invention encodes a Glck protein, the sequence of which is selected from the group consisting of SEQ ID NO: 25 and any Glck variant sequence having at least 90% similarity or identity with SEQ ID NO:25 as defined herein (in particular SEQ ID NO: 26, 27, 28, 29, 30, 31, 32, 33 or 34), wherein the amino acid of the glucokinase corresponding to position 275 of SEQ ID NO:25 (or the amino acid at position 275 of the glucokinase) is not an acidic amino acid.

In a particular embodiment, either as the SEQ ID NO:25 or any GlcK variant sequence having at least 90% similarity or identity with SEQ ID NO: 25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is selected from the group consisting of lysine and any of its conservative amino acids; thus, in some embodiments, the codon 275 of the glcK gene carried by the Streptococcus thermophilus strain of the invention is a codon encoding an amino acid selected from the group consisting of lysine and any of its conservative amino acids; thus, in some embodiments, the glcK gene carried by the Streptococcus thermophilus strain of the invention encodes a Glck protein, the sequence of which is selected from the group consisting of SEQ ID NO: 25 and any Glck variant sequence having at least 90% similarity or identity with SEQ ID NO: 25 as defined herein (in particular SEQ ID NO: 26, 27, 28, 29, 30, 31, 32, 33 or 34), wherein the amino acid of the glucokinase corresponding to position 275 of SEQ ID NO: 25 (or the amino acid at position 275 of the glucokinase) is a lysine and any of its conservative amino acids.

In a particular embodiment, either as the SEQ ID NO:25 or any Glck variant sequence having at least 90% similarity or identity with SEQ ID NO:25 as defined herein (in particular SEQ ID NO: 26, 27, 28, 29, 30, 31, 32, 33 or 34), the amino acid of the glucokinase corresponding to position 275 of SEQ ID NO:25 (or the amino acid at position 275 of the glucokinase) is a lysine; thus, in some embodiments, the codon 275 of the glcK gene carried by the Streptococcus thermophilus strain of the invention is a codon encoding a lysine respectively, in particular is AAA or AAG, respectively; thus, in a particular embodiment, the sequence of the Glck protein of a lactose-positive, sucrose-negative Streptococcus thermophilus strain of the invention is selected from the group consisting of SEQ ID NOs: 22, 35, 36, 37, 38, 39, 40, 41, 42 and 43; thus, in some embodiments, the glcK gene carried by the Streptococcus thermophilus strain of the invention encodes a GlcK protein, the sequence of which is selected from the group consisting of SEQ ID NOs: 22, 35, 36, 37, 38, 39, 40, 41, 42 and 43; in a particular embodiment, the glcK gene carried by the Streptococcus thermophilus strain of the invention is as defined in SEQ ID NO: 21.

In another embodiment, the sequence of the Glck protein of a lactose-positive, sucrose-negative Streptococcus thermophilus strain of the invention is selected from the group consisting of SEQ ID NOs: 46, 47, 48, 49, 50, 51, 52, 53, 54 and 55, wherein the amino acid at position 144 of said variant is any amino acid except a glycine, in particular is any amino acid except an aliphatic amino acid, in particular is a serine; thus, in some embodiments, the glcK gene carried by the Streptococcus thermophilus strain of the invention encodes a Glck protein, the sequence of which is selected from the group consisting of SEQ ID NOs: 46, 47, 48, 49, 50, 51, 52, 53, 54 and 55, wherein the amino acid at position 144 of the glucokinase is not a glycine, in particular is not an aliphatic amino acid, in particular is a serine.

In a particular embodiment, either as the SEQ ID NO:46 or any Glck variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is not a glycine; thus, in some embodiments, the codon 144 of the glcK gene carried by the Streptococcus thermophilus strain of the invention is not GGT, GGC, GGA or GGG; thus, in some embodiments, the glcK gene carried by the Streptococcus thermophilus strain of the invention encodes a Glck protein, the sequence of which is selected from the group consisting of SEQ ID NO: 46 and any GlcK variant sequence having at least 90% similarity or identity with SEQ ID NO: 46 as defined herein (in particular SEQ ID NO: 47, 48, 49, 50, 51, 52, 53, 54 or 55), wherein the amino acid of the glucokinase corresponding to position 144 of SEQ ID NO: 46 (or the amino acid at position 144 of the glucokinase) is not a glycine.

In a particular embodiment, either as the SEQ ID NO:46 or any Glck variant sequence having at least 90% similarity or identity with SEQ ID NO: 46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is not an aliphatic amino acid; thus, in some embodiments, the codon 144 of the glcK gene carried by the Streptococcus thermophilus strain of the invention is a codon which does not encode an aliphatic amino acid; thus, in some embodiments, the glcK gene carried by the Streptococcus thermophilus strain of the invention encodes a GlcK protein, the sequence of which is selected from the group consisting of SEQ ID NO:46 and any Glck variant sequence having at least 90% similarity or identity with SEQ ID NO:46 as defined herein (in particular SEQ ID NO: 47, 48, 49, 50, 51, 52, 53, 54 or 55), wherein the amino acid of the glucokinase corresponding to position 144 of SEQ ID NO:46 (or the amino acid at position 144 of the glucokinase) is not an aliphatic amino acid.

In a particular embodiment, either as the SEQ ID NO:46 or any Glck variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is selected from the group consisting of serine and any of its conservative amino acids; thus, in some embodiments, the codon 144 of the glcK gene carried by the Streptococcus thermophilus strain of the invention is a codon encoding an amino acid selected from the group consisting of serine and any of its conservative amino acids; thus, in some embodiments, the glcK gene carried by the Streptococcus thermophilus strain of the invention encodes a GlcK protein, the sequence of which is selected from the group consisting of SEQ ID NO: 46 and any GlcK variant sequence having at least 90% similarity or identity with SEQ ID NO: 46 as defined herein (in particular SEQ ID NO: 47, 48, 49, 50, 51, 52, 53, 54 or 55), wherein the amino acid of the glucokinase corresponding to position 144 of SEQ ID NO:46 (or the amino acid at position 144 of the glucokinase) is a serine and any of its conservative amino acids.

In a particular embodiment, either as the SEQ ID NO:46 or any GlcK variant sequence having at least 90% similarity or identity with SEQ ID NO:46 as defined herein (in particular SEQ ID NO: 47, 48, 49, 50, 51, 52, 53, 54 or 55), the amino acid of the glucokinase corresponding to position 144 of SEQ ID NO:46 (or the amino acid at position 144 of the glucokinase) is a serine; thus, in some embodiments, the codon 144 of the glcK gene carried by the Streptococcus thermophilus strain of the invention is a codon encoding a serine, in particular is AAA or AAG; thus, in a particular embodiment, the sequence of the Glck protein of a lactose-positive, sucrose-negative Streptococcus thermophilus strain of the invention is selected from the group consisting of SEQ ID NOs: 45, 56, 57, 58, 59, 60, 61, 62, 63 and 64; thus, in some embodiments, the glcK gene carried by the Streptococcus thermophilus strain of the invention encodes a Glck protein, the sequence of which is selected from the group consisting of SEQ ID NOs: 45, 56, 57, 58, 59, 60, 61, 62, 63 and 64; in a particular embodiment, the glcK gene carried by the Streptococcus thermophilus strain of the invention is as defined in SEQ ID NO:44.

When defining the sequence of the Glck protein of a lactose-positive, sucrose-negative Streptococcus thermophilus strain of the invention, it is according to the teaching of this application that the glucokinase activity in the strain expressing this GlcK protein is significantly reduced but not null as defined herein and optionally that the Vmax of the glucokinase in this strain is significantly reduced but not null as defined herein.

Mutations of a Gene Encoding a Protein of the Mannose-Glucose-Specific PTS, in Particular Mutations of the manL, manM and manN Genes.

This part describes mutations of a gene encoding a protein of the mannose-glucose-specific PTS, in particular mutations of the manL, manM and manN genes, which can be used either in combination with a mutation of a glcK gene as defined herein, or in combination with a mutation of a ccpA gene as defined herein, or in combination with both a mutation of a glcK gene and a mutation of a ccpA gene as defined herein, in the context of a lactose-positive, sucrose-negative Streptococcus thermophilus strain of the invention.

Any mutation in a gene encoding a protein of the mannose-glucose-specific PTS is appropriate, may be combined with a mutated glcK gene as defined herein, or combined with a mutation of a ccpA gene as defined herein, or combined with both a mutated glcK gene and a mutated ccpA gene as defined herein in a lactose-positive, sucrose-negative Streptococcus thermophilus strain.

The inventors have shown that mutations in a gene encoding a protein of the mannose-glucose-specific PTS, in particular in a mutated manL gene, a mutated manM gene, or a mutated manN gene, which reduce or abolish the import of glucose from the medium in a lactose-positive, sucrose-negative Streptococcus thermophilus strain, are particularly advantageous within the invention. In some embodiments, the mutation of the gene encoding a protein of the mannose-glucose-specific PTS leads to the reduction or abolition of the glucose import activity of the protein encoded by this gene. In some embodiments, the mutated gene is the manL gene and the mutation of the man gene leads to the reduction or abolition of the glucose import activity of the EIIAB^{Man} protein. In some embodiments, the mutated gene is the manM gene and the mutation of the manM gene leads to the reduction or abolition of the glucose import activity of the EIIC^{Man} protein. In some embodiments, the mutated gene is the manN gene and the mutation of the manN gene leads to the reduction or abolition of the glucose import activity of the EIID^{Man} protein.

In some embodiments, the mutation of the gene encoding a protein of the mannose-glucose-specific PTS, in particular of the manL gene, manM gene or manN gene, is a mutation leading to the knock-out (i.e., the complete disruption) of the gene.

In some embodiments, the mutation of the gene encoding a protein of the mannose-glucose-specific PTS, in particular of the manL gene, manM gene or manN gene, is a mutation of the promoter of the gene, in particular a mutation of the promoter of the gene reducing or inhibiting the transcription of the gene.

In some embodiments, the mutation of the gene encoding a protein of the mannose-glucose-specific PTS, in particular of the manL gene, manM gene or manN gene, is a mutation introduced into the coding sequence of the gene, in particular a mutation leading to the reduction or abolition of the glucose import activity of the protein encoded by the mutated gene, in particular to the reduction or abolition of the glucose import activity of the EIIAB^{Man} protein, EIIC^{Man} protein or EIID^{Man} protein.

In some embodiments, the mutation of the gene encoding a protein of the mannose-glucose-specific PTS, in particular of the manL gene, manM gene or manN gene is a mutation in the coding sequence of the gene, leading to a truncated protein, in particular to a truncated EIIAB^{Man} protein, a truncated EIIC^{Man} protein or a truncated EIID^{Man} protein, in particular to a truncated protein (such as a truncated EIIAB^{Man} protein, a truncated EIIC^{Man} protein or a truncated EIID^{Man} protein) having a reduced or abolished glucose import activity. Whatever the position of the truncation, the mutation introduced into the gene is either a nucleotide substitution leading to a STOP codon or a deletion, insertion or deletion/insertion leading to a frameshift of the open reading frame and a premature STOP codon. In some embodiments, the mutation introduced into the gene is a nucleotide substitution leading to a STOP codon. In some embodiments, the mutation introduced into the gene is a deletion, insertion or deletion/insertion leading to a frameshift of the open reading frame and a premature STOP codon.

Though two Streptococcus thermophilus strains may differ by the sequence of their respective manL, manM or manN gene, this does not necessarily mean that one of these genes is mutated in the sense of the invention. Indeed, not considered as mutations of the manL, manM or manN gene gene within the present invention are:
variations at the nucleotide level which do not lead to any change at the protein level (silent variation) and which do not impact the translation of the manL, manM or manN RNA; and
variations at the nucleotide level which do lead to a change at the protein level, but wherein this change does not enable, when combined with a mutated glcK gene as defined herein, or combined with a mutation of a ccpA gene as defined herein, or when combined with both a mutated glcK gene and a mutated ccpA gene in a lactose-positive, sucrose-negative phenotype of a Streptococcus thermophilus strain.

Non-limitative examples of manL, manM and manN genes (respectively encoding the EIIAB^{Man} protein, the EIIC^{Man} protein and the EIID^{Man} protein) which are not considered as mutated in the sense of the present invention are:
the polynucleotide encoding the EIIAB^{Man} protein as defined in SEQ ID NO: 78, in particular the polynucleotide as defined in SEQ ID NO: 77; this EIIAB^{Man} type is the one of the DSM28255 strain;
the polynucleotide encoding the EIIAB^{Man} protein as defined in SEQ ID NO: 80, 82, 84, 86, 88, 90, 92, 94, 96, 98, 100, 102, 104, 106, 108 and 110, in particular the polynucleotide as defined in SEQ ID NO: 79, 81, 83, 85, 87, 89, 91, 93, 95, 97, 99, 101, 103, 105, 107 and 109. The sequence of the EIIAB^{Man} proteins as defined in SEQ ID NO: 80, 82, 84, 86, 88, 90, 92, 94, 96, 98, 100, 102, 104, 106, 108 and 110 is from 98.4 to 99.6% identical to SEQ ID NO: 78;
the polynucleotide encoding the EIIC^{Man} protein as defined in SEQ ID NO: 130, in particular the polynucleotide as defined in SEQ ID NO: 129; this EIIC^{Man} type is the one of DSM28255;
the polynucleotide encoding the EIIC^{Man} protein as defined in SEQ ID NO: 132, 134, 136, 138, 140, 142, 144, 146, 148, 150, 152, 154 and 156, in particular the polynucleotide as defined in SEQ ID NO: 131, 133, 135, 137, 139, 141, 143, 145, 147, 149, 151, 153 and 155. The sequence of the EIIC^{Man} proteins as defined in SEQ ID NO: 132, 134, 136, 138, 140, 142, 144, 146, 148, 150, 152, 154 and 156 is from 98.5 to 99.6% identical to SEQ ID NO:130;
the polynucleotide encoding the EIID^{Man} protein as defined in SEQ ID NO:167, in particular the polynucleotide as defined in SEQ ID NO: 166; this EIID^{Man} type is the one of DSM28255 strain;
the polynucleotide encoding the EIID^{Man} protein as defined in SEQ ID NO: 169, 171, 173, 175, 177, 179, 181, 183, 185, 187, 189, 191, 193, 195, 197, 199, 201, 203 and 205, in particular the polynucleotide as defined in SEQ ID NO: 168, 170, 172, 174, 176, 178, 180, 182, 184, 186, 188, 190, 192, 194, 196, 198, 200, 202 and 204. The sequence of the EIID^{Man} proteins as defined in SEQ ID NO: 169, 171, 173, 175, 177, 179, 181, 183, 185, 187, 189, 191, 193, 195, 197, 199, 201, 203 and 205 is from 97.3 to 99.6% identical to SEQ ID NO:167.

The present inventors have identified at least one mutation in the manL gene, which when inserted into the manL gene of an original lactose-positive, sucrose-negative Streptococcus thermophilus strain [mutated in the glcK gene, the ccpA gene or both the glcK and ccpA genes as defined herein] enables an interesting phenotype within the present invention.

In some embodiments, the mutation in the manL gene leads to the truncation of the EIIAB^{Man} protein at position 305. In some embodiments, the mutation in the manL gene is the substitution of the nucleotide G in the nucleotide T at position 916 (leading to a stop codon at position 306). A Streptococcus thermophilus EIIAB^{Man} protein truncated at position 305 is referred herein as IIAB^{Man}₃₀₅.

In some embodiments, the sequence of said EIIAB^{Man} protein truncated in position 305 is selected from the group consisting of:
a) a sequence as defined in SEQ ID NO: 112; and
b) a EIIAB^{Man} variant sequence having at least about 90%, such as at least 95% similarity or identity with SEQ ID NO: 112, in particular being 305 amino acids in length.

For the definition of the EIIAB^{Man} variant having at least about 90%, such as at least 95% similarity or identity with SEQ ID NO: 112, the similarity or identity is calculated herein over the whole length of the 2 sequences after optimal alignment [i.e., number of similar or identical amino acid residues in the aligned parts(s) of the sequences]. In a particular embodiment, the EIIAB^{Man} variant sequence has at least 91, 92, 93, 94, 95, 96, 97, 98 or 99% similarity or identity with SEQ ID NO: 112.

In a particular embodiment, the EIIAB^{Man} variant sequence differs from SEQ ID NO: 112 by from 1 to 30 amino acid substitutions. In a particular embodiment, the EIIAB^{Man} variant sequence differs from SEQ ID NO: 112 by from 1 to 20 amino acid substitutions. In a particular embodiment, the EIIAB^{Man} variant sequence differs from SEQ ID NO: 112 by from 1 to 15 amino acid substitutions. In a particular embodiment, the EIIAB^{Man} variant sequence differs from SEQ ID NO: 112 by from 1 to 10 amino acid substitutions. In a particular embodiment, the EIIAB^{Man} variant sequence differs from SEQ ID NO: 112 by 1, 2, 3, 4, 5, 6, 7, 8, 9 or 10 amino acid substitutions. In some embodiments, the sequence of the EIIAB^{Man} protein of a lactose-positive, sucrose-negative Streptococcus thermophilus strain of the invention is selected from the group consisting of SEQ ID NOs: 112 to 128.

In some embodiments, the manL gene carried by the Streptococcus thermophilus strain of the invention encodes a EIIAB^{Man} protein, the sequence of which is selected from the group consisting of SEQ ID NO: 112 and any EIIAB^{Man} variant sequence having at least about 90%, such as at least 95% similarity or identity with SEQ ID NO: 112 as defined herein (in particular SEQ ID NO: 113 to 128). In some embodiments, the manL gene carried by the Streptococcus thermophilus strain of the invention is as defined in SEQ ID NO:111.

The inventors have identified at least one mutation in the manM gene, which when inserted into the manM gene of an original lactose-positive, sucrose-negative Streptococcus thermophilus strain [mutated in the glcK gene, the ccpA gene or both the glcK and ccpA genes as defined herein] enables some interesting properties for these strains.

In some embodiments, the mutation in the manM gene leads to the truncation of the EIIC^{Man} protein at position 208. In some embodiments, the mutation in the manM gene is the substitution of the nucleotide G in the nucleotide T at position 625 (leading to a stop codon at position 209). A Streptococcus thermophilus EIIC^{Man} protein truncated at position 208 is referred herein as EIIC^{Man208}.

In some embodiments, the sequence of said EIIC^{Man} protein truncated in position 208 is selected from the group consisting of:
a) a sequence as defined in SEQ ID NO: 158; and
b) a EIIC^{Man} variant sequence having at least 90% similarity or identity with SEQ ID NO: 158, in particular being 208 amino acids in length.

For the definition of the EIIC^{Man} variant having at least 90% similarity or identity with SEQ ID NO: 158, the similarity or identity is calculated herein over the whole length of the 2 sequences after optimal alignment [i.e., number of similar or identical amino acid residues in the aligned parts(s) of the sequences]. In a particular embodiment, the EIIC^{Man} variant sequence has at least 91, 92, 93, 94, 95, 96, 97, 98 or 99% similarity or identity with SEQ ID NO: 158.

In a particular embodiment, the EIIC^{Man} variant sequence differs from SEQ ID NO: 158 by from 1 to 30 amino acid substitutions. In a particular embodiment, the EIIC^{Man} variant sequence differs from SEQ ID NO: 158 by from 1 to 20 amino acid substitutions. In a particular embodiment, the EIIC^{Man} variant sequence differs from SEQ ID NO: 158 by from 1 to 15 amino acid substitutions. In a particular embodiment, the EIIC^{Man} variant sequence differs from SEQ ID NO: 158 by from 1 to 10 amino acid substitutions. In a particular embodiment, the EIIC^{Man} variant sequence differs from SEQ ID NO: 158 by 1, 2, 3, 4, 5, 6, 7, 8, 9 or 10 amino acid substitutions. In some embodiments, the sequence of the EIIC^{Man} protein of a lactose-positive, sucrose-negative Streptococcus thermophilus strain of the invention is selected from the group consisting of SEQ ID NO: 158 to 165.

In some embodiments, the manM gene carried by the Streptococcus thermophilus strain of the invention encodes a EIIC^{Man} protein, the sequence of which is selected from the group consisting of SEQ ID NO: 158 and any EIIC^{Man} variant sequence having at least about 90%, such as at least 95% similarity or identity with SEQ ID NO: 158 as defined herein (in particular SEQ ID NO: 159 to 165). In some embodiments, the manM gene carried by the Streptococcus thermophilus strain of the invention is as defined in SEQ ID NO: 157.

In some embodiments, the mutation in the manM gene is an insertion of one nucleotide C at any one of positions 438, 439, or 440 of SEQ ID NO: 129 creating a frameshift in manM, leading to a stop codon at position 180 of SEQ ID NO: 130 (manM₁₂₉₉₇). A resulting Streptococcus thermophilus IIC^{Man} protein truncated at position 179 is referred herein as IIC^{Man179} In some embodiments, the sequence of said IIC^{Man} protein truncated in position 179 is selected from the group consisting of:
a) a sequence as defined in SEQ ID NO: 223; and
b) a IIC^{Man} variant sequence having at least 90% similarity or identity with SEQ ID NO: 223,
in particular being 179 amino acids in length.

For the definition of the IIC^{Man} variant having at least 90% similarity or identity with SEQ ID NO: 223, the similarity or identity is calculated herein over the whole length of the 2 sequences after optimal alignment [i.e., number of similar or identical amino acid residues in the aligned parts(s) of the sequences]. In a particular embodiment, the IIC^{Man} variant sequence has at least 91, 92, 93, 94, 95, 96, 97, 98 or 99% similarity or identity with SEQ ID NO: 223.

In a particular embodiment, the IIC^{Man} variant sequence differs from SEQ ID NO:223 by from 1 to 30 amino acid substitutions. In a particular embodiment, the IIC^{Man} variant sequence differs from SEQ ID NO: 223 by from 1 to 20 amino acid substitutions. In a particular embodiment, the IIC^{Man} variant sequence differs from SEQ ID NO: 223 by from 1 to 15 amino acid substitutions. In a particular embodiment, the IIC^{Man} variant sequence differs from SEQ ID NO: 223 by from 1 to 10 amino acid substitutions. In a particular embodiment, the IIC^{Man} variant sequence differs from SEQ ID NO: 223 by 1, 2, 3, 4, 5, 6, 7, 8, 9 or 10 amino acid substitutions. In some embodiments, the sequence of the IIC^{Man} protein of a lactose-positive Streptococcus thermophilus strain used according to the invention is selected from the group consisting of SEQ ID NO: 223.

In some embodiments, the manM gene carried by the Streptococcus thermophilus strain used according to the invention encodes a IIC^{Man} protein, the sequence of which is selected from the group consisting of SEQ ID NO: 223 and any IIC^{Man} variant sequence having at least about 90%, such as at least 95% similarity or identity with SEQ ID NO: 223 as defined herein.

The inventors have identified at least one mutation in the manN gene, which when inserted into the manN gene of an original lactose-positive, sucrose-negative Streptococcus thermophilus strain [mutated in the glcK gene, the ccpA gene or both the glcK and ccpA genes as defined herein] enables some interesting properties for these strains of Streptococcus thermophilus.

In some embodiments, the mutation in the manN gene leads to the truncation of the EIID^{Man} protein at position 28. In some embodiments, the mutation in the manN gene is an insertion of a nucleotide A in the stretch of 5 nucleotides A at positions 37-41 (leading to a stretch of 6 nucleotides A, a frameshift of the open reading frame and a truncation of the EIID^{Man} protein at position 28). This Streptococcus thermophilus EIID^{Man} protein truncated at position 28 is referred herein as IID^{Man}₂₈.

In some embodiments, the sequence of said EIID^{Man} protein truncated in position 28 is selected from the group consisting of:
a) a sequence as defined in SEQ ID NO: 207; and
b) an EIID^{Man} variant sequence having at least 90% similarity or identity with SEQ ID NO: 207, in particular being 28 amino acids in length.

For the definition of the EIID^{Man} variant having at least 90% similarity or identity with SEQ ID NO: 207, the similarity or identity is calculated herein over the whole length of the 2 sequences after optimal alignment [i.e., number of similar or identical amino acid residues in the aligned parts(s) of the sequences]. In a particular embodiment, the EIIC^{Man} variant sequence has at least 91, 92, 93, 94, 95, 96, 97, 98 or 99% similarity or identity with SEQ ID NO: 207.

In a particular embodiment, the EIID^{Man} variant sequence differs from SEQ ID NO: 207 by from 1 to 10 amino acid substitutions. In a particular embodiment, the EIID^{Man} variant sequence differs from SEQ ID NO: 207 by 1, 2, 3, 4, 5, 6, 7, 8, 9 or 10 amino acid substitutions. In some embodiments, the sequence of the EIID^{Man} protein of a lactose-positive, sucrose-negative Streptococcus thermophilus strain of the invention is selected from the group consisting of SEQ ID NO: 207 to 211.

In some embodiments, the manN gene carried by the Streptococcus thermophilus strain of the invention encodes an EIID^{Man} protein, the sequence of which is selected from the group consisting of SEQ ID NO: 207 and any EIID^{Man} variant sequence having at least 90% similarity or identity with SEQ ID NO: 207 as defined herein (in particular SEQ ID NO: 208 to 211). In some embodiments, the manN gene carried by the Streptococcus thermophilus strain of the invention is as defined in SEQ ID NO:206.

Whatever the mutation of the glcK gene as defined herein and the mutation of the ccpA gene as defined herein (present alone or combined in a lactose-positive, sucrose-negative, Streptococcus thermophilus strain of the invention), at least one gene encoding a protein of the mannose-glucose-specific PTS is mutated as defined herein. Whatever the embodiment, the invention encompasses a lactose-positive, sucrose-negative, Streptococcus thermophilus strain carrying a mutation in one, two or three genes selected from the group consisting of the manL gene, the manM gene and the manN gene. In some embodiments, the lactose-positive, sucrose-negative, Streptococcus thermophilus strain of the invention carries a mutation in manL. In some embodiments, the lactose-positive, sucrose-negative, Streptococcus thermophilus strain of the invention carries a mutation in manM. In some embodiments, the lactose-positive, sucrose-negative, Streptococcus thermophilus strain of the invention carries a mutation in manN. In some embodiments, the lactose-positive, sucrose-negative, Streptococcus thermophilus strain of the invention carries a mutation in manL and a mutation in manM. In some embodiments, the lactose-positive, sucrose-negative, Streptococcus thermophilus strain of the invention carries a mutation in manL and a mutation in manN. In some embodiments, the lactose-positive, sucrose-negative, Streptococcus thermophilus strain of the invention carries a mutation in manM and a mutation in manN. In some embodiments, the lactose-positive, sucrose-negative, Streptococcus thermophilus strain of the invention carries a mutation in manL, a mutation in manM and a mutation in manN.

Any method can be used to identify a mutation in a gene encoding a protein of the mannose-glucose-specific PTS, in particular in the manL gene, manM gene or manN gene suitable within the lactose-positive, sucrose-negative Streptococcus thermophilus strain of the invention.

As an example, to identify a suitable mutation in the manL gene, manM gene or manN gene, the person skilled in the art can proceed by the following method:
a) provide the DSM32587 strain (mutated in its glcK gene)
b) carry out mutagenesis on the manL, manM or manN gene of the strain in a), for example by random or directed mutagenesis, to obtain a manL, manM or manN gene, the sequence of which is different from the sequence of the manL, manM or manN gene of DSM32587, to obtain a man-mutated DSM32587 strain;
c) determining if a pH_{STOP} phenotype is accomplished.

Alternatively, the person skilled in the art can proceed by the following method:
a) Provide a parent strain, such as the DSM28255 strain in which its ccpA gene has been replaced by the mutated ccpA gene as defined in SEQ ID NO: 71 (ccpA_{Δ1A114-120}), called herein DGCC7710-ccpA_{Δ1A114-120} strain;
b) carry out mutagenesis on the manL, manM or manN gene of the strain in a), for example by random or directed mutagenesis, to obtain a manL, manM or manN gene, the sequence of which is different from the sequence of the manL, manM or manN gene of the DGCC7710-ccpA_{Δ1A114-120} strain, to obtain a man-mutated DGCC7710-ccpA_{Δ1A114-120} strain;
c) determining if a pH_{STOP} phenotype is accomplished.

Once identified, the mutated manL, manM or manN gene according to the invention can be introduced in lieu of the manL, manM or manN of a lactose-positive, sucrose-negative, Streptococcus thermophilus strain, to obtain a lactose-positive, sucrose-negative, Streptococcus thermophilus strain of the invention.

Mutations of the ccpA Gene

This part describes mutations of the ccpA gene which can be used either in combination with a mutation of a gene encoding a protein of the mannose-glucose-specific PTS as defined herein, or in combination with a mutation of a gene encoding a protein of the mannose-glucose-specific PTS as defined herein and a mutation of the glcK gene as defined herein, in the context of a lactose-positive, sucrose-negative Streptococcus thermophilus strain of the invention.

Any mutation in the ccpA gene is appropriate, as long as when combined with a mutated gene encoding a protein of the mannose-glucose-specific PTS as defined herein, or when combined with both a mutated glcK gene as defined herein and a mutated gene encoding a protein of the mannose-glucose-specific PTS as defined herein in a lactose-positive, sucrose-negative Streptococcus thermophilus strain, a pH_{STOP} phenotype is obtained, when said strain is used to ferment milk.

For the determination of the ratio of the beta-galactosidase activity over the glucokinase activity in the strain of the invention, the Beta-galactosidase Activity Assay described herein and The Glucokinase Activity Assay as described herein are used:

### The Beta-Galactosidase Activity Assay:

During milk fermentation, the lactose contained in the milk (as the main carbohydrate source in milk) is imported into Streptococcus thermophilus strains. The intracellular lactose is then cleaved into glucose and galactose by the beta-galactosidase enzyme (such that 1 mole of lactose gives 1 mole of glucose and 1 mole of galactose), which activity is also referred to as the beta-galactosidase activity.

The beta-galactosidase activity in a Streptococcus thermophilus strain of the invention is assayed by the Beta-galactosidase Activity Assay [carried out using the Streptococcus thermophilus strain of the invention].

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) after 3 hours of growth on M17+30 g/l lactose at 42° C., washed in 1.5 ml cold lysis buffer (KPO4 0.1 M), and resuspended in 300 μl cold lysis buffer. EDTA-free protease inhibitors “cOmplete™” (Roche, supplier reference 04693132001) is added in lysis buffer as described by the provider. Cells are disrupted by the addition of 100 mg glass beads (150-212 μm, Sigma G1145) to 250 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The beta-galactosidase activity in the cell extracts is determined spectrophotometrically by a monitoring of the hydrolysis of O-nitro-Phenol-Beta-Glactoside (ONPG) into galactose and O-nitro-phenol (ONP). 20 μL of the bacteria extract are mixed with 135 μL of React Buffer (NaPO₄ 0.1 M+KCl 0.01 M+MgSO₄ 0.001 M+ONPG 3 mM+Beta Mercapto Ethanol 60 mM, pH=6). The production of ONP leads to a yellow color into the tube. When this color appears, the reaction is block by adding 250 μL of Stopping buffer (Na₂CO₃ 1 M). The optical density at 420 nm is measured using a Synergy HT multi-detection microplate reader (BIO-TEK). One unit of galactosidase corresponds to the amount of enzyme that catalyzes the production of 1 μmole ONP per minute under the assay conditions. Beta-Galactosidase activity is calculated as follows:

Beta-Galactosidase activity (U/g of total protein extract)=dOD×V/[dt×l×ε×Qprot], wherein:
dOD is the variation of optical density (OD) at 420 nm between the blank and the tested sample
V is the volume of the reaction in which the optical density is measured (herein 250 μL)
dt=represent the duration in minutes between the addition of the 20 μL of bacterial extract and the addition of the 250 L stopping buffer
l=optical path length (herein 0.73 cm)
ε=molar attenuation coefficient of ONP (herein 4500 cm²/μmol)
Qprot=quantity of protein in the cuvette (in g)
   Measurements are at least triplicated for each sample, and the beta-galactosidase specific activity values given herein under the Beta-galactosidase Activity Assay described herein are the mean of three independent experiments.

### The Glucokinase Activity Assay:

The glucokinase activity in a Streptococcus thermophilus strain of the invention, for the determination of the ratio, is assayed as follows using the Streptococcus thermophilus strain of the invention.

A fresh overnight culture of the Streptococcus thermophilus strain to be assayed in M17 containing 30 g/L lactose is obtained and used to inoculate at 1% (vol/vol) 10 ml of fresh M17 30 g/L lactose. Cells are harvested by centrifugation (6000 g, 10 min, 4° C.) after 3 hours of growth on M17+30 g/L lactose at 42° C., washed in 1.5 ml cold GLCK buffer (5 mM MgCl2, 10 mM K₂HPO₄/KH₂PO₄ [pH 7.2]), and resuspended in 300 μl cold GLCK buffer. EDTA-free protease inhibitors “cOmplete™” (Roche, supplier reference 04693132001) is added in GLCK buffer as described by the provider. Cells are disrupted by the addition of 100 mg glass beads (150-212 μm, Sigma G1145) to 250 μl resuspended cells and oscillation at a frequency of 30 cycles/s for 6 min in a MM200 oscillating mill (Retsch, Haan, Germany). Cell debris and glass beads are removed by centrifugation (14000 g, 15 min, 4° C.), and supernatant transferred into a clean 1.5 mL centrifuge tube kept on ice. Total protein content is determined by using the FLUKA Protein Quantification Kit-Rapid (ref 51254). The glucokinase activity in the cell extracts is determined spectrophotometrically by a glucose-6-phosphate dehydrogenase (G-6PDH, EC1.1.1.49):NADPH-coupled assay (Porter et al., 1982), essentially as described by Pool et al. (2006). Each sample (5, 10 and 20 μL) is added to assay buffer (10 mM K₂HPO₄/KH₂PO₄ [pH 7.2], 5 mM MgCl2, 1 mM ATP, 20 mM glucose, 1 mM NADP, 1 U G-6PDH) in a 250 μL final volume, and the mixture was left for 5 min at 30° C. The optical density at 340 nm is measured for 5 minutes by using a Synergy HT multi-detection microplate reader (BIO-TEK). One unit of glucokinase corresponds to the amount of enzyme that catalyzes the phosphorylation of 1 μmole of D-glucose to D-Glucose 6-phosphate per minute under the assay conditions. Glucokinase activity is calculated as follows:

Glucokinase activity (U/g of total protein extract)=dOD×V/[dt×l×ε×Qprot], wherein:
dOD is the variation of optical density (OD) at 340 nm
V is the volume of the reaction (herein 250 μL)
dt=measurement time (in minutes)
l=optical path length (herein 0.73 cm)
ε=molar attenuation coefficient of NADPH; H⁺ (herein 6220 cm2/μmol)
Qprot=quantity of protein in the cuvette (in g)
   Measurements are triplicated for each sample, and the glucokinase specific activity values given herein under The Glucokinase Activity Assay are the mean of three independent experiments.

In some embodiments, the ccpA gene mutation is not a mutation leading to the knock-out (i.e., the complete disruption) of the gene.

In some embodiments, the ccpA gene mutation is a mutation in the coding sequence of the ccpA gene, in particular in the first 270 nucleotides of the coding sequence of the ccpA gene. In some embodiments, the mutation is a mutation selected from the group consisting of:
a) a non-sense mutation (i.e. leading to a STOP codon) located between the nucleotide 1 and the nucleotide 270 of the coding sequence of the ccpA gene; and
b) a mutation, located in the first quarter of the coding sequence of the ccpA gene (i.e., between nucleotide 1 and the nucleotide 250), leading to a frameshift of the open reading frame of the ccpA gene.

In some embodiments, the mutation leading to a frameshift of the open reading frame of the ccpA gene is located between nucleotide 50 and the nucleotide 200 of the coding sequence of the ccpA gene. In some embodiments, the mutation leading to a frameshift of the open reading frame of the ccpA gene is located between nucleotide 100 and the nucleotide 150 of the coding sequence of the ccpA gene. Whatever the location of the mutation leading to a frameshift, the mutation is selected from the group consisting of a deletion, an insertion or a deletion/insertion (which all are not a multiple of 3).

Though two Streptococcus thermophilus strains may differ by the sequence of their respective ccpA gene, this does not necessarily mean that one of these two ccpA genes is mutated in the sense of the invention. Indeed, not considered as mutations of the ccpA gene within the present invention are variations at the nucleotide level which do lead to a change at the protein level.

Non-limitative examples of ccpA genes which are not considered as mutated in the sense of the invention are:
the polynucleotide as defined in SEQ ID NO: 65 (ccpA type ST1); this ccpA type is the one of the DSM28255 strain;
the polynucleotide as defined in SEQ ID NO: 66 (ccpA type ST2), which has 99.8% identity with SEQ ID NO: 65;
the polynucleotide as defined in SEQ ID NO: 67 (ccpA type ST3), which has 99.8% identity with SEQ ID NO: 65;
the polynucleotide as defined in SEQ ID NO: 68 (ccpA type ST4), which has 99.7% identity with SEQ ID NO: 65;
the polynucleotide as defined in SEQ ID NO: 69 (ccpA type ST5), which has 99.8% identity with SEQ ID NO: 65; and
the polynucleotide as defined in SEQ ID NO: 70 (ccpA type ST6), which has 99.7% identity with SEQ ID NO: 65.

The invention may in some embodiments be directed to a lactose-positive, sucrose-negative Streptococcus thermophilus strain carrying a mutation in the ccpA gene selected from the group consisting of a non-sense mutation located between the nucleotide 1 and the nucleotide 270 of the coding sequence of the ccpA gene and a mutation, located in the first quarter of the coding sequence of the ccpA gene, leading to a frameshift of the open reading frame of the ccpA gene.

In some embodiments, the mutation of the ccpA gene is a deletion of a nucleotide A in the stretch of 7 nucleotides A at positions 114-120 (leading to a frameshift of the open reading frame of the ccpA gene). Such Streptococcus thermophilus mutated ccpA gene is referred herein as ccpA_{Δ1A114-120}.

In some embodiments, the sequence of said ccpA gene with a STOP codon at codon 66 is selected from the group consisting of:
a) a sequence as defined in SEQ ID NO:71; and
b) a ccpA variant sequence having at least 90% identity with SEQ ID NO: 71. The ccpA variant as defined herein carries a mutation as defined above, i.e., is selected from the group consisting of a non-sense mutation located between the nucleotide 1 and the nucleotide 270 of the coding sequence of the ccpA gene and a mutation, located in the first quarter of the coding sequence of the ccpA, leading to a frameshift of the open reading frame of the ccpA gene.

For the definition of the ccpA variant having at least 90% identity with SEQ ID NO: 71, the identity is calculated herein over the whole length of the 2 sequences after optimal alignment [i.e., number of identical nucleotides in the aligned parts(s) of the sequences]. In a particular embodiment, the ccpA variant sequence has at least 91, 92, 93, 94, 95, 96, 97, 98 or 99% identity with SEQ ID NO:71. In a particular embodiment, the ccpA variant sequence differs from SEQ ID NO: 71 by from 1 to 30 nucleotide substitutions. In a particular embodiment, the ccpA variant sequence differs from SEQ ID NO: 71 by from 1 to 20 nucleotide substitutions. In a particular embodiment, the ccpA variant sequence differs from SEQ ID NO:71 by from 1 to 15 nucleotide substitutions. In a particular embodiment, the ccpA variant sequence differs from SEQ ID NO: 71 by from 1 to 10 nucleotide substitutions. In a particular embodiment, the ccpA variant sequence differs from SEQ ID NO: 71 by 1, 2, 3, 4, 5, 6, 7, 8, 9 or 10 nucleotide substitutions. In some embodiments, the sequence of the ccpA gene of a lactose-positive, sucrose-negative Streptococcus thermophilus strain of the invention is selected from the group consisting of SEQ ID NOs: 71, 72, 73, 74, 75 and 76.

The person skilled in the art is given, in this part of the application, guidance on how to identify mutations of the ccpA gene other than the one specifically disclosed.

Thus, the person skilled in the art can proceed by the following method:
a) Provide a parent strain, such as the DSM28255 strain in which its manL gene has been replaced by the mutated manL gene as defined in SEQ ID NO: 111 (manL gene encoding the IIAB^{Man}₃₀₅ protein), called herein DGCC7710-IIAB^{Man}₃₀₅ strain;
b) carry out mutagenesis on the ccpA gene of the strain in a), for example by random or directed mutagenesis, to obtain a ccpA the sequence of which is different from the sequence of the ccpA gene of the DGCC7710-IIAB^{Man}₃₀₅ strain, to obtain a ccpA-mutated DGCC7710-IIAB^{Man}₃₀₅ strain.

Alternatively, the person skilled in the art can proceed by the following method:
a) Provide a parent strain, such as the DSM28255 strain in which its manM gene has been replaced by the mutated manM gene as defined in SEQ ID NO: 157 (manM gene encoding the EIIC^{Man} 208 protein), called herein DGCC7710-IIC^{Man}₂₀₈ strain;
b) carry out mutagenesis on the ccpA gene of the strain in a), for example by random or directed mutagenesis, to obtain a ccpA the sequence of which is different from the sequence of the ccpA gene of the DGCC7710-IIC^{Man}₂₀₈ strain, to obtain a ccpA-mutated DGCC7710-IIC^{Man}₂₀₈ strain.

Alternatively, the person skilled in the art can proceed by the following method:
a) Provide a parent strain, such as the DSM28255 strain in which its manM gene has been replaced by the mutated manM gene, which mutation is creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇);
b) carry out mutagenesis on the ccpA gene of the strain in a), for example by random or directed mutagenesis, to obtain a ccpA the sequence of which is different from the sequence of the ccpA gene of the DGCC7710-IIC^{Man}₂₀₈ strain, to obtain a ccpA-mutated DGCC7710-IIC^{Man}₂₀₈ strain.

The person skilled in the art can also proceed by the following method:
a) Provide a parent strain, such as the DSM28255 strain in which its manN gene has been replaced by the mutated manN gene as defined in SEQ ID NO: 206 (manN gene encoding the IID^{Man}₂₈ protein), called herein DGCC7710-IID^{Man}₂₈ strain;
b) carry out mutagenesis on the ccpA gene of the strain in a), for example by random or directed mutagenesis, to obtain a ccpA the sequence of which is different from the sequence of the ccpA gene of the DGCC7710-IID^{Man}₂₈ strain, to obtain a ccpA-mutated DGCC7710-IID^{Man}₂₈ strain.

Once identified, a mutated ccpA gene—as identified herein—can be introduced in lieu of the ccpA gene of a lactose-positive, sucrose-negative Streptococcus thermophilus strain, to obtain a lactose-positive, sucrose-negative Streptococcus thermophilus strain of the invention.

### Further Characterization of the Strains of the Invention

The present inventors have shown that strains having a mutation in a gene encoding a protein of the mannose-glucose-specific PTS and a mutation in the glcK gene and/or the ccpA gene) enable, when they are used to ferment milk (both plain and sucrose-added milk):
1) to obtain a low lactose fermented milk; and/or
2) to obtain a fermented milk not undergoing post-acidification, even when stored at fermentation temperature.

In some embodiments, the lactose-positive, sucrose-negative Streptococcus thermophilus strain of the invention is characterized by the fact that the strain leads to a low lactose fermented milk, when used to ferment milk.

In some embodiments, the lactose-positive, sucrose-negative Streptococcus thermophilus strain of the invention is characterized by the fact that the strain leads to a fermented milk, not undergoing post-acidification when stored at fermentation temperature.

In some embodiments, the lactose-positive, sucrose-negative Streptococcus thermophilus strain of the invention is characterized by the fact that the strain leads to a low lactose fermented milk not undergoing post-acidification when stored at fermentation temperature (when used to ferment milk).

The expression “low-lactose fermented milk” means a fermented milk which has an amount of remaining lactose in the fermented milk, at the end of the fermentation as described in Example 5, which is less than 60 mM, less than 50 mM, less than 45 mM, less than 40 mM, less than 35 mM or less than 30 mM. It is noteworthy that the amount of lactose in the milk measured according to Example 6, is prior to fermentation about 50 g/L.

The expression “not undergoing post-acidification” means a milk product which, when inoculated with a strain of the invention and fermented as described in Example 5, has its pH decreased to a specific pH value (i.e. the pH_{STOP} value) at which value the speed of acidification definitively becomes less than 0.1 mUpH/min, wherein said pH_{STOP} value is between 4.4 and 5.3, and optionally the slope between pH6 and PH 5.5 is at least-0.008 UpH/min.

The term “pH_{STOP} phenotype” as used herein refers to a strain of Streptococcus thermophilus, which when used in fermenting milk as described in Example 5 will produce a milk product “not undergoing post-acidification” as defined herein.

Thus, the absence of post-acidification is characterized by the fact that the pH of the fermented milk stops between 4.4 and 5.3. The pH is considered to be stopped (pH_{STOP}), when the speed of acidification (ΔpH/Δtime) definitively becomes less than 0.1 mUpH/min (less than 0.0001 UpH/min). By “definitively becomes”, it is meant that the speed of acidification stays less than 0.1 mUpH/min for the remaining time of the fermentation described in Example 5 (i.e. up to 24 h at fermentation temperature), once the pH_{STOP} is obtained.

In some embodiments, the pH_{STOP} obtained using a strain of the invention is between 4.7 and 5.2. In some embodiments, the pH_{STOP} obtained using a strain of the invention is between 4.8 and 5.1. In some embodiments, the pH_{STOP} obtained using a strain of the invention is between a minimal value selected from the group consisting of 4.4, 4.5, 4.6, 4.7 and 4.8 and a maximal value selected from the group consisting of 5.1, 5.2 and 5.3.

In some embodiments, the fermented milk not undergoing post-acidification is also characterized by the slope between pH6 and pH5.5. The slope represents the inverse of the velocity (speed of acidification). In some embodiments, the slope is at least −0.009 UpH/min. In some embodiments, the slope is at least −0.01 UpH/min.

Composition, Method and Use with Lactose-Positive, Sucrose-Negative Streptococcus thermophilus Strains of the Invention

The invention is also directed to a bacterial composition comprising or consisting of at least one, in particular one, lactose-positive, sucrose-negative, Streptococcus thermophilus strain of the invention. In a particular embodiment, the bacterial composition is a pure culture, i.e., comprises or consists of a single bacterium strain. In another embodiment, the bacterial composition is a mixed culture, i.e., comprises or consists of a lactose-positive, sucrose-negative Streptococcus thermophilus strain(s) of the invention and at least one other bacterium strain. By “at least” (in reference to a strain or bacterium), it is meant 1 or more, and in particular 1, 2, 3, 4 or 5 strains.

Thus, in some embodiments, a bacterial composition of the invention comprises or consists of a lactose-positive, sucrose-negative Streptococcus thermophilus strain(s) of the invention and at least one lactic acid bacterium of the species selected from the group consisting of a Lactococcus species, a Streptococcus species, a Lactobacillus species including Lactobacillus acidophilus, an Enterococcus species, a Pediococcus species, a Leuconostoc species, a Bifidobacterium species and an Oenococcus species or any combination thereof. Lactococcus species include Lactobacillus acidophilus and Lactococcus lactis, including Lactococcus lactis subsp. lactis, Lactococcus lactis subsp. cremoris and Lactococcus lactis subsp. lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus delbrueckii subsp. bulgaricus, and Lactobacillus helveticus.

In some embodiments, the bacterial composition comprises or consists of a lactose-positive, sucrose-negative Streptococcus thermophilus strain(s) of the invention, and at least one Streptococcus thermophilus strain, different from the S. thermophilus strain(s) of the invention and/or at least one strain of the Lactobacillus species, and/or any combination thereof. In a particular embodiment, the bacterial composition comprises or consists of the Streptococcus thermophilus strain(s) of the invention, one or several strain(s) of the species Lactobacillus delbrueckii subsp. bulgaricus and/or one or several strain(s) of the species Lactobacillus helveticus and/or any combination thereof, and optionally at least one Streptococcus thermophilus strain, different from the S. thermophilus strain(s) of the invention. In a particular embodiment, the bacterial composition comprises or consists of the Streptococcus thermophilus strain(s) of the invention, at least one strain of species Streptococcus thermophilus, different from the S. thermophilus strain(s) of the invention, and a strain of the species Lactobacillus delbrueckii subsp. bulgaricus. In another particular embodiment, the bacterial composition comprises or consists of the Streptococcus thermophilus strain(s) of the invention, and a strain of the species Lactobacillus delbrueckii subsp. bulgaricus.

In some embodiments, the bacterial composition comprises or consists of the Streptococcus thermophilus strain(s) of the invention, a Lactococcus lactis subsp. lactis and/or a Lactococcus lactis subsp. cremoris.

In a particular embodiment of any bacterial composition defined herein, either as a pure or mixed culture, the bacterial composition further comprises at least one probiotic strain such as Bifidobacterium animalis subsp. lactis, Lactobacillus acidophilus, Lactobacillus paracasei, or Lactobacillus casei.

In a particular embodiment, the bacterial composition, either as a pure or mixed culture as defined above is under frozen, dried, freeze-dried, liquid or solid format, in the form of pellets or frozen pellets, or in a powder or dried powder. In a particular embodiment, the bacterial composition of the invention is in a frozen format or in the form of pellets or frozen pellets, in particular contained into one or more box or sachet. In another embodiment, the bacterial composition as defined herein is under a powder form, such as a dried or freeze-dried powder, in particular contained into one or more box or sachet.

In a particular embodiment, the bacterial composition of the invention, either as a pure culture or mixed culture as defined above, and whatever the format (frozen, dried, freeze-dried, liquid or solid format, in the form of pellets or frozen pellets, or in a powder or dried powder) comprises a lactose-positive, sucrose-negative Streptococcus thermophilus strain(s) of the invention in a concentration comprised in the range of 10⁵ to 10¹² cfu (colony forming units) per gram of the bacterial composition. In a particular embodiment, the concentration of a lactose-positive, sucrose-negative Streptococcus thermophilus strain(s) within the bacterial composition of the invention is in the range of 10⁷ to 10¹² cfu per gram of the bacterial composition, and in particular at least 10⁷, at least 10⁸, at least 10⁹, at least 10¹⁰ or at least 10¹¹ CFU/g of the bacterial composition. In a particular embodiment, when in the form of frozen or dried concentrate, the concentration of a lactose-positive, sucrose-negative Streptococcus thermophilus strain(s)—as pure culture or as a mixed culture-within the bacterial composition is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

The invention also concerns a method for manufacturing a fermented product, comprising a) inoculating a substrate with a lactose-positive, sucrose-negative Streptococcus thermophilus strain(s) of the invention and b) fermenting said inoculated substrate, to obtain a fermented product. In a particular embodiment, a lactose-positive, sucrose-negative Streptococcus thermophilus strain(s) of the invention is inoculated as a bacterial composition as defined herein, such as a pure culture or a mixed culture. In some embodiments, the substrate into which the S. thermophilus strain(s) or bacterial composition of the invention is added to is milk substrate. By “milk substrate”, it is meant milk of animal and/or plant origin. In a particular embodiment, the milk substrate is of animal origin, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. Therefore, in a particular embodiment, the invention also provides a method for manufacturing a fermented dairy product, comprising a) inoculating a milk substrate with a lactose-positive, sucrose-negative Streptococcus thermophilus strain(s) or bacterial composition of the invention and b) fermenting said inoculated milk substrate, to obtain a fermented dairy product.

The invention is also directed to the use of the lactose-positive, sucrose-negative, Streptococcus thermophilus strain(s) of the invention or a composition of the invention, to manufacture a fermented dairy product.

The invention is also directed to a fermented dairy product, which is obtained using a lactose-positive, sucrose-negative Streptococcus thermophilus strain(s) of the invention or a bacterial composition of the invention, in particular obtained or obtainable by the method of the invention. Thus, the invention is directed to a fermented dairy product comprising a lactose-positive, sucrose-negative Streptococcus thermophilus strain(s) of the invention. In a particular embodiment, the fermented dairy food product of the invention is fresh fermented milk.

Various features and embodiments of the present invention will now be described by way of non-limiting examples.

### Examples

### Assays Used in the Following Examples

### 1. Test A:

S. thermophilus strains to be tested were cultivated overnight at 37° C. in 10 mL of M17 medium (Thermo Scientific™ Oxoid™, CM0785) containing 30 g/L of lactose. Cells from each culture were harvested by centrifugation (2100 g for 10 min at 4° C.), the cell pellet was resuspended into 10 mL fresh sugar-free M17 medium. One hundred microliters of serial 10-fold dilutions of the cell suspension were used to seed the surface of agar plates of M17 medium containing 30 g/L of either lactose or sucrose. Upon incubation at 37° C. under anaerobic conditions (in a sealed container using the Anaerocult® A system (Merck, Darmstadt, Germany)) for 24 hours, colonies grown on lactose and on sucrose were numerated. The ratio of colonies forming (cfu) on sucrose (sucrose-positive colonies) compare to lactose (lactose-positive colonies) was calculated.

### 2. Test B:

S. thermophilus strains to be tested were cultivated overnight at 37° C. in 10 mL of M17 medium (Thermo Scientific™ Oxoid™, CM0785) containing 30 g/L of lactose. Cells from each culture were harvested by centrifugation (2100 g for 10 min at 4° C.), the cell pellet was resuspended into 10 mL fresh sugar-free M17 medium. Two hundred microliters of the cell suspension were used to inoculate 10 mL of M17 medium containing no sugar or 30 g/L of sugar (either lactose or glucose). Two hundred microliters of the inoculated medium from each of the three conditions (sugar-free, lactose or glucose) were distributed in triplicate in 96-wells microtiter-plate and 100 μL of paraffine oil were added on the top of each well. Microtiter-plates were incubated at 37° C. Bacterial growth was monitored during 24 h using a SpectraMax ID5 (Molecular Devices, San Jose) at 600 nm (reads every 15 min), with orbital shaking for 5 sec at high speed before each read. For the purpose of evaluating the ability of the strains to grow on glucose as a sole source of carbohydrate, the speed of growth on glucose (μ_{glucose}) was calculated. To ensure proper comparison between strains with limiting the impact of inoculation rate, this value was calculated at a timepoint corresponding to the maximal growth rate of the strain on lactose (μmax_{lactose}). In addition, the ratio corresponding to μ_{glucose} over μmax_{lactose} was determined.

### 3. Test C:

S. thermophilus strains to be tested were cultivated overnight at 37° C. in 10 ml of M17 (Thermo Scientific™ Oxoid™, CM0785) containing 30 g/L of lactose. Cells from each culture were harvested by centrifugation (2100 g for 10 min at 4° C.), the cell pellet was resuspended into 10 mL fresh sugar-free M17 medium. Two hundred microliters of the cell suspension were used to seed 10 mL of M17 medium containing 30 g/L of either lactose or sucrose. Upon incubation at 37° C. for 24 hours, the OD₆₀₀ of the cultures were measured at 600 nm using a Biochrom WPA Biowave DNA spectrophotometer (Biochrom Ltd., Cambridge, UK) and compared.

### Numbered Embodiments of the Invention

1. A lactose-positive, sucrose-negative, Streptococcus thermophilus strain carrying one or more mutations selected from the group consisting of:
1) one or more mutation in one or more gene of the sucrose regulon; which mutation provides for a significant reduction in the ability to grow on and/or transport sucrose by said strain; and which mutation at least comprises
   i) one or more of the scrA gene, and/or the scrB gene and/or the scrR gene, and
   ii) optionally a further mutation in the promoter region regulating the expression of the scrA, scrB and/or scrR genes, such as a mutation that protect the strain from reversing from the sucrose negative phenotype; and
2) optionally one or more further mutation affecting the glucose porter, such as affecting glcu and/or its expression, which mutation restores or improves glucose consumption of said strain when grown on glucose as the sole source of carbohydrates.

2. The lactose-positive, sucrose-negative, Streptococcus thermophilus strain according to embodiment 1, which strain is glucose-positive or glucose slow-growing.

3. The lactose-positive, sucrose-negative, Streptococcus thermophilus strain according to embodiments 1 or 2, which strain has one or more mutation in the sucrose regulon in one or more of the scrA gene and the scrB gene.

4. The lactose-positive, sucrose-negative, Streptococcus thermophilus strain according to embodiment 3, which mutation in one or more gene of the sucrose regulon is in the scrA gene effectively disrupting gene function, such as an insertion or deletion resulting in a frameshift of the open reading frame of the gene coding for the EIIABC^{Sucrose} protein leading to the translation of a truncated EIIABC^{Sucrose} protein encoded by the scrA gene, such as a mutation by insertion or deletion of a nucleotide, such as at or around a nucleotide corresponding to position 133 of SEQ ID NO: 212, in the open-reading-frame (ORF) of the scrA gene.

5. The lactose-positive, sucrose-negative, Streptococcus thermophilus strain according to any one of embodiments 1-4, which strain has a mutation in the promoter region regulating the expression of any one of the scrA, scrB and/or scrR genes.

6. The lactose-positive, sucrose-negative, Streptococcus thermophilus strain according to embodiment 5, which mutation is a SNP in the promoter region of the scrA and/or scrB genes, such as a SNP in the promoter region of the scrB gene, such as a mutation near the −10 and 35 sequence of the promoter, such as a mutation of T to G corresponding to position 38 of SEQ ID NO: 213 upstream of the scrB promoter, such as a mutation that will comprise the entire sequence identified by SEQ ID NO:213 or a variant sequence being at least about 90%, such as at least about 95% identical herewith (pscrB₈₂₉).

7. The lactose-positive, sucrose-negative, Streptococcus thermophilus strain according to any one of embodiments 1-6, which strain has a mutation affecting the glucose porter, which mutation restores or improves glucose consumption of said strain when grown on glucose as the sole source of carbohydrates, such as a full recovery of the growth on glucose as compared to that of a lactose-positive and sucrose-positive Streptococcus thermophilus parental strain.

8. The lactose-positive, sucrose-negative, Streptococcus thermophilus strain according to embodiment 7, which mutation affecting the glucose porter is affecting glcU and/or its expression, such as located within the promoter region of glcU, such as mutations selected from
a) an insertion of a T nucleotide in the poly-T region at position corresponding to position-75 of the glcU promoter identified as SEQ ID NO: 216 (pglcU^{IN_T}), such as a mutation that will contain a T nucleotide at position 102 of SEQ ID NO: 217, such as a mutation comprising the entire sequence identified by SEQ ID NO:217 or a variant sequence being at least about 90%, such as at least about 95% identical herewith;
b) an insertion of a C nucleotide in the poly-T region at position corresponding to position-75 of the glcU promoter identified as SEQ ID NO: 216 (pglcU^{IN_C}), such as a mutation that will contain a C nucleotide at position 102 of SEQ ID NO: 218, such as a mutation that will comprise the entire sequence identified by SEQ ID NO: 218 or a variant sequence being at least about 90%, such as at least about 95% identical herewith;
c) a mutation of a T to a C nucleotide in the poly-T region at position corresponding to position-81 of the glcU promoter identified as SEQ ID NO: 216 (pglcU^{SNP_C}), such as a mutation that will contain a C nucleotide at position 96 of SEQ ID NO: 219, such as a mutation that will comprise the entire sequence identified by SEQ ID NO:219 or a variant sequence being at least about 90%, such as at least about 95% identical herewith;
d) an insertion of a sequence at a position corresponding to between nucleotide position 110 and 111 of the glcU promoter identified as SEQ ID NO:216, such as an insertion of a sequence identified by nucleotide 1 to 1321 of SEQ ID NO: 221, such as a mutation that will comprise the entire sequence identified by SEQ ID NO: 220 or a variant sequence being at least about 90%, such as at least about 95% identical herewith or SEQ ID NO: 221 or a variant sequence being at least about 90%, such as at least about 95% identical herewith (pglcU^{IS}).

9. The lactose-positive, sucrose-negative, Streptococcus thermophilus strain according to any one of embodiments 1-8, further comprising one or more mutations selected from:
a) a mutation in the glcK gene encoding a glucokinase, the glucokinase activity of which in said strain is significantly reduced, but not null; and/or
b) a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇); and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167.

10. The lactose-positive, sucrose-negative, Streptococcus thermophilus strain according to any one of embodiments 1-9, which strain is galactose-negative, such as by mutation in the glcK gene and/or in the ccpA gene optionally combined with a mutation in one of the genes encoding a protein of the man-nose-glucose-specific PTS.

11. The lactose-positive, sucrose-negative, Streptococcus thermophilus strain according to embodiments 9-10, which strain has a mutation in the glcK gene encoding a glucokinase, which mutation provides for the glucokinase activity in said strain being significantly reduced but not null.

12. The lactose-positive, sucrose-negative, Streptococcus thermophilus strain according to any one of embodiments 9-11, wherein said mutated glcK gene encodes a glucokinase selected from the group consisting of:
a) a glucokinase having an amino acid at position 275 which is not a glutamic acid, in particular which is not an acidic amino acid, in particular which is a lysine,
b) a glucokinase having an amino acid at position 275 which is not a glutamic acid, in particular which is not an acidic amino acid, in particular which is a lysine (glcK₃₇₈), and having an arginine at position 278 and/or a serine at position 279; and
c) a glucokinase having an amino acid at its position 144 which is not a glycine, in particular which is not an aliphatic amino acid, in particular which is a serine.

13. The lactose-positive, sucrose-negative, Streptococcus thermophilus strain according to any one of embodiments 9-12, wherein said mutated glcK gene encodes a glucokinase, the sequence of which is selected from the group consisting of:
a) a sequence as defined in SEQ ID NO: 25, wherein the amino acid at position 275 is not a glutamic acid, in particular is not an acidic amino acid, in particular is a lysine; and
b) a Glck variant sequence having at least 90% similarity or identity with SEQ ID NO: 25, wherein the amino acid of said glucokinase corresponding to position 275 of SEQ ID NO:25 (or the amino acid at position 275 of said glucokinase) is not a glutamic acid, in particular is not an acidic amino acid, in particular is a lysine;
c) a sequence as defined in SEQ ID NO: 46, wherein the amino acid at position 144 is not a glycine, in particular is not an aliphatic amino acid, in particular is a serine; and
d) a GlcK variant sequence having at least 90% similarity or identity with SEQ ID NO:46, wherein the amino acid of said glucokinase corresponding to position 144 of SEQ ID NO:46 (or the amino acid at position 144 of said glucokinase) is not a glycine, in particular is not an aliphatic amino acid, in particular is a serine.

14. The lactose-positive, sucrose-negative, Streptococcus thermophilus strain according to any one of embodiments 1-13, further carrying a mutation in the ccpA gene.

15. The lactose-positive, sucrose-negative, Streptococcus thermophilus strain according to any one of embodiments 1-14, further carrying a mutation in the gene encoding a protein of the mannose-glucose-specific PTS, such as a gene selected from the group consisting of the manL gene, the manM gene and the manN gene.

16. The lactose-positive, sucrose-negative, Streptococcus thermophilus strain according to embodiment 14 or 15, wherein the ccpA gene carries a mutation selected from the group consisting of a non-sense mutation located between the nucleotide 1 and the nucleotide 270 of the coding sequence of the ccpA gene and a mutation, located in the first quarter of the coding sequence of the ccpA gene, leading to a frameshift of the open reading frame of the ccpA gene.

17. The lactose-positive, sucrose-negative, Streptococcus thermophilus strain according to embodiment 16, wherein the sequence of said mutated ccpA gene is selected from the group consisting of:
a) a sequence as defined in SEQ ID NO: 71; and
b) a ccpA variant sequence having at least 90%, such as at least 95% identity with SEQ ID NO: 71.

18. The lactose-positive, sucrose-negative, Streptococcus thermophilus strain according to any one of embodiments 1-17, wherein, when said strain is able to ferment milk as described in Example 5 during 24 hours of fermentation:
a) the concentration of lactose remaining in the fermented milk is less than 60 mM; and/or
b) the pH of the milk decreases to a pH at which the speed of acidification definitively becomes less than 0.1 mUpH/min (pH_{STOP}), wherein said pH_{STOP} is between 4.4 and 5.3, and optionally the slope between pH6 and pH5.5 is at least-0.008 UpH/min.

19. The lactose-positive, sucrose-negative, Streptococcus thermophilus strain according to any one of embodiments 1-18, wherein said strain has the ability to overconsume lactose as compared to a parental strain without said one or more mutations, such as wherein the remaining amount of lactose when fermenting sweet milk as described and assayed according to Example 6 is lower than 100 mM, such as lower than 90 mM, 80 mM, 70 mM, 60 mM, 50 mM, 40 mM, 30 mM, 20 mM, 10 mM, 8 mM, 6 mM, 4 mM, 2 mM or 1 mM.

20. The lactose-positive, sucrose-negative, Streptococcus thermophilus strain according to any one of embodiments 1-19, wherein said strain has the ability to release an increased amount of glucose as compared to a parental strain without said one or more mutations, such as wherein the glucose release as described and assayed according to Example 6 is higher than 10 mM, such as higher than 20 mM, 30 mM, 40 mM, 50 mM, 60 mM, 70 mM, 80 mM, or 90 mM.

21. The lactose-positive, sucrose-negative, Streptococcus thermophilus strain according to any one of embodiments 1-20, wherein said strain is releasing a concentration of glucose assayed as described in example 6, which is at least 50 mM, such as at least 60, 70, 80 or 90 mM or releasing a concentration of glucose which is increased of at least 150% or at least 200%, such as 300%, 400%, 500% as compared to the glucose concentration released by the parent strain, when both assayed as described in example 6.

22. A composition comprising at least one, in particular one, lactose-positive, sucrose-negative, Streptococcus thermophilus strain according to any one of embodiments 1-21, in particular in combination with another lactic acid bacteria, in particular with one or more strain(s) selected from the group consisting of a strain of the Lactobacillus genus, such as a Lactobacillus delbrueckii subsp bulgaricus strain, a strain of the Lactococcus genus, such as a Lactococcus lactis strain or a strain of the Bifidobacterium genus.

23. A method for manufacturing a fermented dairy product, in particular a fermented milk, comprising inoculating a milk substrate with the lactose-positive, sucrose-negative, Streptococcus thermophilus strain according to any one of embodiments 1-21, or a composition according to embodiment 19, and fermenting said inoculated milk, to obtain a fermented dairy product.

24. Use of the lactose-positive, sucrose-negative, Streptococcus thermophilus strain according to any one of embodiments 1-21, or a composition according to embodiment 22, to manufacture a fermented dairy product.

25. A fermented dairy product comprising at least one, in particular one, lactose-positive, sucrose-negative, Streptococcus thermophilus strain according to any one of embodiments 1-21, or as obtained by a method according to embodiment 23.

26. A Streptococcus thermophilus strain, such as a lactose-positive, sucrose-negative Streptococcus thermophilus strain selected from the group consisting of:
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34132;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34134;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34138;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34139;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34140;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34146;—a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34147; and
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 16 Feb. 2022 under number DSM34172.

27. A lactose-positive, sucrose-negative, Streptococcus thermophilus strain selected from the group consisting of:
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the man gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO:71 or a ccpA variant sequence having at least about 90%, such as at least 95% identity with SEQ ID NO:71 at least 95%;
a strain of a Streptococcus thermophilus, such as the strain deposited under the Budapest Treaty on Jan. 14, 2014 in the name of Danisco Deutschland GmbH at Leibniz-Institut DSMZ under number DSM34172 [herein referred to as ST1 or DSM34172 strain] comprising one or more of the following features:
   1) into which the sequence of the open-reading-frame (ORF) of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
   6) having a ccpA sequence of SEQ ID NO:71 or a ccpA variant sequence having at least about 90%, such as at least 95% identity with SEQ ID NO: 71 at least 95%;
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO: 71 or a ccpA variant sequence having at least about 90%, such as at least 95% identity with SEQ ID NO: 71 at least 95%;
a strain of a Streptococcus thermophilus, such as the strain deposited under the Budapest Treaty on Jan. 14, 2014 in the name of Danisco Deutschland GmbH at Leibniz-Institut DSMZ under number DSM28255 [herein referred to as ST4 or DSM28255 strain] comprising one or more of the following features:
   1) into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}),
   2) the sequence upstream of the start codon of scrB gene is replaced by the corresponding sequence as set forth in SEQ ID NO:213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
   6) having a ccpA sequence of SEQ ID NO:71 or a ccpA variant sequence having at least about 90%, such as at least 95% identity with SEQ ID NO: 71 at least 95%;
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO:25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO:71 or a ccpA variant sequence having at least about 90%, such as at least 95% identity with SEQ ID NO: 71;
a strain of a Streptococcus thermophilus, such as the strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS})
   2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
   6) having a ccpA sequence of SEQ ID NO: 71 or a ccpA variant sequence having at least about 90%, such as at least 95% identity with SEQ ID NO: 71;
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS})
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of the scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO:167; and optionally further comprising a ccpA sequence of SEQ ID NO: 71 or a ccpA variant sequence having at least about 90%, such as at least 95% identity with SEQ ID NO: 71;
a strain of a Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D38124 Braunschweig-Germany] comprising one or more of the following features:
   1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS})
   2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
   3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
   4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
   5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
   6) having a ccpA sequence of SEQ ID NO:71 or a ccpA variant sequence having at least about 90%, such as at least 95% identity with SEQ ID NO: 71.

28. A polynucleotide comprising one or more mutation in one or more gene of the sucrose regulon, which one or more mutation provides for a significant reduction in the ability to uptake and consume any sucrose in a strain selected from:
a) a strain corresponding to the Streptococcus thermophilus strain deposited under the Budapest Treaty on Jan. 14, 2014 in the name of Danisco Deutschland GmbH at Leibniz-Institut DSMZ under number DSM28255, into which strain said polynucleotide is replacing the corresponding sequence in DSM28255;
b) a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132, into which strain said polynucleotide is replacing the corresponding sequence in DSM34132;
c) a strain corresponding to the Streptococcus thermophilus strain deposited under accession number DSM33651 on Sep. 29, 2020, at the DSMZ [Deutsche Sammlung von Mikroorganismen und Zellkulturen GmbH, Inhoffenstrasse 7B, D-38124 Braunschweig-Germany], into which strain said polynucleotide is replacing the corresponding sequence in DSM33651;
d) a strain corresponding to the Streptococcus thermophilus strain deposited under the Budapest Treaty on 16 Feb. 2022 in the name of DuPont Nutrition Biosciences ApS at Leibniz-Institut DSMZ under number DSM34172, into which strain said polynucleotide is replacing the corresponding sequence in DSM34172.

29. A polynucleotide comprising one or more mutation affecting the glucose porter, which one or more mutation restores or improves glucose consumption of a strain selected from:
a) a strain corresponding to the Streptococcus thermophilus strain deposited under the Budapest Treaty on Jan. 14, 2014 in the name of Danisco Deutschland GmbH at Leibniz-Institut DSMZ under number DSM28255, into which strain said polynucleotide is replacing the corresponding sequence in DSM28255;
b) a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132, into which strain said polynucleotide is replacing the corresponding sequence in DSM34132,
c) a strain corresponding to the Streptococcus thermophilus strain deposited under accession number DSM33651 on Sep. 29, 2020, at the DSMZ [Deutsche Sammlung von Mikroorganismen und Zellkulturen GmbH, Inhoffenstrasse 7B, D-38124 Braunschweig-Germany], into which strain said polynucleotide is replacing the corresponding sequence in DSM33651;
d) a strain corresponding to the Streptococcus thermophilus strain deposited under the Budapest Treaty on 16 Feb. 2022 in the name of DuPont Nutrition Biosciences ApS at Leibniz-Institut DSMZ under number DSM34172, into which strain said polynucleotide is replacing the corresponding sequence in DSM34172.

30. A polynucleotide according to any one of embodiments 28 or 29, which one or more mutation is as identified in any one of embodiments 3-8.

31. A method for the selection of a lactose-positive, sucrose-negative, Streptococcus thermophilus strain, which method includes the steps of:
1) providing a strain of Streptococcus thermophilus including
   a) one or more mutation in one or more gene of the sucrose regulon; which mutation provides for a significant reduction in the ability to grow on and/or transport sucrose by said strain; and which mutation at least comprises
      i) one or more of the scrA gene, and/or the scrB gene and/or the scrR gene, and
      ii) optionally a further mutation in the promoter region regulating the expression of the scrA, scrB and/or scrR genes, such as a mutation that protect the strain from reversing from the sucrose negative phenotype;
2) screen for a strain of Streptococcus thermophilus from step 1), which strain has a significant reduction in the ability to grow on and transport sucrose; and
3) optionally screen prior to, simultaneously with, or after any one of steps 1 and 2 for a Streptococcus thermophilus strain which has a restored or improved glucose consumption, optionally from one or more further mutation affecting the glucose porter, such as affecting glcU and/or its expression.

32. The method according to embodiment 31, which strain is as defined in any one of embodiments 1-21.

### EXAMPLES

### Example 1: Identification of Mutations in the scrA Gene and in the Promoter of the scrB Gene

Streptococcus thermophilus is described as a species able to grow both on lactose (lactose-positive phenotype) and on sucrose (sucrose-positive phenotype). A proprietary strain collection was screened for strains of S. thermophilus unable to grow on sucrose as a sole source of carbohydrate. S. thermophilus strains to be tested were cultivated overnight at 37° C. in M17 (Thermo Scientific™ Oxoid™, CM0785) containing 30 g/L lactose. Cells from each culture were harvested by centrifugation (2100 g for 10 min at 4° C.), the cell pellet was washed twice with Tryptone Salt (TS; tryptone 1 g/L, NaCl 8.5 g/L) then resuspended into 10 mL fresh sugar-free M17 medium. Washed cells were used to inoculate in triplicate in 96-wells microtiter-plate at 1% (v/v) 200 μL of fresh M17 medium containing 30 g/L of sugar (either lactose or sucrose) and 100 μL of paraffine oil was added on the top of each well. Microtiter-plates were incubated at 37° C. Bacterial growth was monitored during 24 h using a SpectraMax ID5 (Molecular Devices, San Jose) at 620 nm (reads every 15 min), with orbital shaking for 5 sec at high speed before each read. One lactose-positive strain, DGCC8368, was found to be unable to grow on sucrose as a sole source of carbohydrate. Ability to grow on sucrose was defined as the ability of a strain to reach an OD₆₀₀ superior to 0.5 upon 24 hours of incubation at 37° C.

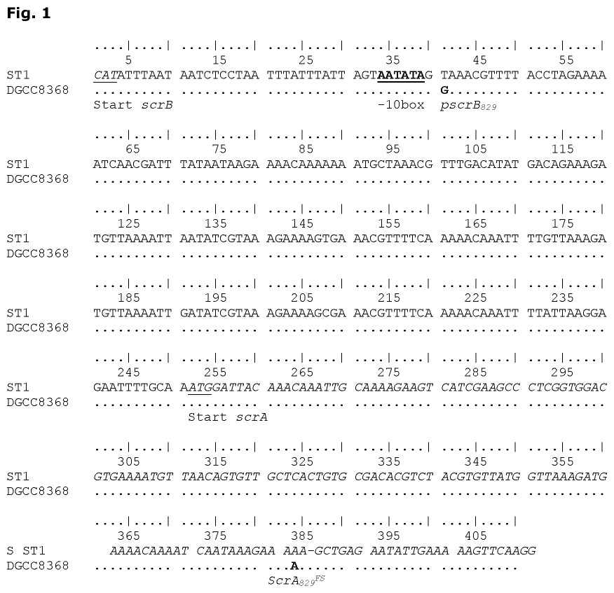

Sequencing of several genes of DGCC8368 (scrA, scrB, scrK and scrR genes), known to be involved in the catabolism of sucrose in S. thermophilus, was performed and compared to corresponding gene sequences of other S. thermophilus strains. First, an insertion of one A nucleotide in a poly-A region was identified at position 133 of the open-reading-frame (ORF) of scrA of DGCC8368 (FIG. 1; SEQ ID NO: 212); the mutation was named scrA₈₂₉^{FS}. This insertion results in a frameshift leading to the translation in a truncated EIIABC^{Sucrose} protein. In addition, a substitution of a nucleotide A by a C nucleotide was identified at position-38 upstream of the start codon of scrB gene (position 38 of SEQ ID NO:213). The mutation was named pscrB₈₂₉ and is located 2 nucleotides upstream of the putative-10 box (FIG. 1). None of these two mutations were found in the scrA gene sequence or the scrB promoter sequence of the other S. thermophilus strains from the collection or in public databases.

### Example 2. Impact of scrA₈₂₉^{FS} on the Growth of S. thermophilus Strains on Sucrose as a Sole Source of Carbohydrate

In order to assess the impact of the scrA₈₂₉^{FS} mutation on the ability of S. thermophilus strains to grow on sucrose, derivatives of S. thermophilus strains ST1 (DSM34172), ST2 (DSM34132) and ST3 (DSM33651), were constructed into which this mutation replaced the native gene alleles. To evaluate the ability of the strains to grow on sucrose as a sole source of carbohydrate, the constructions were analyzed using Test A. In Test A, S. thermophilus strains to be tested were cultivated overnight at 37° C. in 10 mL of M17 medium (Thermo Scientific™ Oxoid™, CM0785) containing 30 g/L of lactose. Cells from each culture were harvested by centrifugation (2100 g for 10 min at 4° C.), the cell pellet was resuspended into 10 mL fresh sugar-free M17 medium. One hundred microliters of serial 10-fold dilutions of the cell suspension were used to seed the surface of agar plates of M17 medium containing 30 g/L of either lactose or sucrose. Upon incubation at 37° C. under anaerobic conditions (in a sealed container using the Anaerocult® A system (Merck, Darmstadt, Germany) for 24 hours, colonies grown on lactose and on sucrose were numerated. The ratio of colonies forming (cfu) on sucrose (sucrose-positive colonies) compare to lactose (lactose-positive colonies) was calculated. The results are summarized in Table 1.

Mutants bearing the scrA₈₂₉^{FS} mutation (ST1-A, ST2-A and ST3-A) were barely able to grow on sucrose as a sole source of carbohydrate. In test A, the ratio of sucrose-positive colonies over lactose-positive colonies was below 10⁻⁶ for the mutants whereas the ratio was close to 1 for the parental strains. Globally, the results indicated that the scrA₈₂₉^{FS} mutation rendered a S. thermophilus strain sucrose-negative, meaning that less than 0.01% of cfu growing on lactose were able to form a colony on sucrose as a sole source of carbohydrate.

TABLE 1
Analysis of the impact of scrA₈₂₉^{FS} on the ability of S. thermophilus
to grow on sucrose as a sole source of carbohydrate.

| | Engineered | Cell count | Cell count- | Cell count ratio |
|---|---|---|---|---|
| Strain | mutation | sucrose^{a} | lactose^{a} | sucrose/lactose^{a} |
| ST1 | | 1.07 10⁹ | 1.34 10⁹ | 0.80 |
| ST1-A | scrA₈₂₉^{FS} | 7.00 10⁰ | 8.80 10⁸ | 0.8 10⁻⁸ |
| ST2 | | 1.10 10⁹ | 1.10 10⁹ | 1.00 |
| ST2-A | scrA₈₂₉^{FS} | 1.14 10² | 2.04 10⁹ | 5.6 10⁻⁸ |
| ST3 | | 1.40 10⁸ | 2.60 10⁸ | 0.54 |
| ST3-A | scrA₈₂₉^{FS} | 1.00 10² | 4.20 10⁸ | 2.4 10⁻⁷ |

^{a}cell counts were established as described in test A.

### Example 3. Combining scrA₈₂₉^{FS} and pscrB₈₂₉ to Stabilize the Sucrose-Negative Phenotype of S. thermophilus Strains

In Test A, low levels of colonies were observed from cultures of the three scrA₈₂₉^{FS} mutants (ST1-A, ST2-A and ST3-A) on sucrose-containing agar plates (see Example 2, Table 1) suggesting the instability of the sucrose-negative phenotype. This instability was clearly observable when these mutants were cultivated in medium containing sucrose as a sole source of carbohydrate (Test C). In Test C, S. thermophilus strains to be tested were cultivated overnight at 37° C. in 10 mL of M17 containing 30 g/L of lactose. Cells from each culture were harvested by centrifugation (2100 g for 10 min at 4° C.), the cell pellet was resuspended into 10 mL fresh sugar-free M17 medium. Two hundred microliters of the cell suspension were used to seed 10 mL of M17 medium containing 30 g/L of either lactose or sucrose. Upon incubation at 37° C. for 24 hours, the OD₆₀₀ of the cultures were measured at 600 nm using a Biochrom WPA Biowave DNA spectrophotometer (Biochrom Ltd., Cambridge, UK) and compared. Results (Table 2) indicated for the parental strains grown on sucrose-containing M17 medium OD values ranging from 2.8 to 6.0. These values were not significantly different from that measured for the three scrA₈₂₉^{FS} mutants (OD₆₀₀ values ranging from 3.7 to 4.6). However, since it should be expected for strains displaying a sucrose-negative phenotype to display OD₆₀₀ values in Test C close to zero; thus, it is likely that the phenotype related to the scrA₈₂₉^{FS} was instable.

In order to assess the impact of the combination of both mutations scrA₈₂₉^{FS} and pscrB₈₂₉ on the stabilization of the sucrose-negative phenotype, derivatives of the scrA₈₂₉^{FS} mutants were constructed into which pscrB₈₂₉ mutation replaced the native gene allele. The parental strains, the scrA₈₂₉^{FS} single mutants and the scrA₈₂₉^{FS}-pscrB₈₂₉ double mutants were compared using Test C. On the contrary to what was observed for scrA₈₂₉^{FS} single mutants, OD₆₀₀ values for the scrA₈₂₉^{FS}-pscrB₈₂₉ double mutants remained low all over the 24 hours of incubation and OD₆₀₀ never reached values above 0.14. This is clearly demonstrating that the combination of scrA₈₂₉^{FS} and pscrB₈₂₉ mutations allows to stabilize the sucrose-negative phenotype conferred by scrA₈₂₉^{FS} mutation. It is also noticeable that in test A no colonies were observed for the scrA₈₂₉^{FS}-pscrB₈₂₉ double mutants, reversion of the sucrose-negative phenotype was below the threshold of detection (<0.5 10⁻⁸).

TABLE 2
Analysis of the impact of the combination of scrA₈₂₉^{FS} and pscrB₈₂₉ mutations on the
stabilization of the sucrose-negative phenotype of S. thermophilus mutant strains.

| | | Cell count | Cell count | Cell count ratio | OD600 on sucrose |
|---|---|---|---|---|---|
| Strain | Engineered mutation | sucrose^{a} | lactose^{a} | sucrose/lactose^{a} | at 24 hours^{c} |
| ST1 | | 1.07 10⁹ | 1.34 10⁹ | 0.81 | 5.770 +/− 0.155 |
| ST1-A | scrA₈₂₉^{FS} | 7.00 10⁰ | 8.80 10⁸ | 0.9 10⁻⁸ | 4.620 +/− 0.164 |
| ST1-AB | scrA₈₂₉^{FS}, pscrB₈₂₉ | 0 | 1.23 10⁹ | <0.5 10⁻⁸ | 0.090 +/− 0.004 |
| ST2 | | 1.10 10⁹ | 1.10 10⁹ | 1.00 | 5.977 +/− 0.230 |
| ST2-A | scrA₈₂₉^{FS} | 1.14 10² | 2.04 10⁹ | 5.6 10⁻⁸ | 4.313 +/− 0.254 |
| ST2-AB | scrA₈₂₉^{FS}, pscrB₈₂₉ | 0 | 1.35 10⁹ | <0.5 10⁻⁸ | 0.134 +/− 0.004 |
| ST3 | | 1.4 10⁸ | 2.60 10⁸ | 1.10 | 2.840 +/− 0.035 |
| ST3-A | scrA₈₂₉^{FS} | 1.00 10² | 4.20 10⁸ | <0.5 10⁻⁸ | 3.790 +/− 0.096 |
| ST3-AB | scrA₈₂₉^{FS}, pscrB₈₂₉ | 0 | 5.2 10⁸ | <0.5 10⁻⁸ | 0.077 +/− 0.023 |

^{a}cell counts ratio were established as described in Test A;
^{b}OD600 on sucrose at 24 hours were performed as in Test C.

### Example 4. scrA₈₂₉^{FS} Mutation Combined or not with pscrB₈₂₉ Mutation Impairs the Ability of S. thermophilus Strains to Grow on Glucose; pglcU Restores this Ability

Strains of S. thermophilus species are described as naturally able to grow on glucose as a sole source of carbohydrate. This ability was controlled for engineered strains bearing the scrA₈₂₉^{FS} mutation with or without the pscrB₈₂₉ mutation. For this control the Test B was performed. In Test B, S. thermophilus strains to be tested were cultivated overnight at 37° C. in 10 mL of M17 medium (Thermo Scientific™ Oxoid™, CM0785) containing 30 g/L of lactose. Cells from each culture were harvested by centrifugation (2100 g for 10 min at 4° C.), the cell pellet was resuspended into 10 mL fresh sugar-free M17 medium. Two hundred microliters of the inoculated medium from the cell suspension were used to inoculate at 1% (v/v) 10 mL of M17 medium containing no sugar or 30 g/L of sugar (either lactose or glucose). Two hundred micro-millimeters of each condition (sugar-free, lactose or glucose) were distributed in triplicate in 96-wells microtiter-plate and 100 μL of paraffine oil were added on the top of each well. Microtiter-plates were incubated at 37° C. Bacterial growth was monitored during 24 h using a SpectraMax ID5 (Molecular Devices, San Jose) at 600 nm (reads every 15 min), with orbital shaking for 5 sec at high speed before each read.

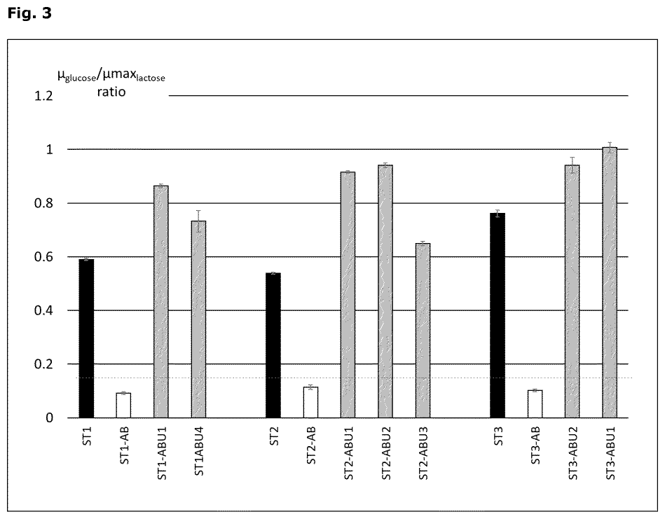

For the purpose of evaluating the ability of the strains to grow on glucose as a sole source of carbohydrate, the speed of growth on glucose (μ_{glucose}) was calculated. To ensure proper comparison between strains with limiting the impact of inoculation rate, this value was calculated at a timepoint corresponding to the maximal growth rate of the strain on lactose (μmax_{lactose}). In addition, the ratio corresponding to μ_{glucose} over μmax_{lactose} was determined. Data are summarized in Table 3 and FIG. 3 provides a graphical comparison of the μ_{glucose}/μmax_{lactose} ratios. Typically, for the lactose-positive, glucose-positive parental S. thermophilus strains, this ratio was superior to 0.5 indicating that strains grew well on glucose even if more slowly than on lactose. Surprisingly, strains bearing the scrA₈₂₉^{FS} mutation with or without the pscrB₈₂₉ mutation were affected in their ability to grow on glucose compared to their parents. Indeed, the values of the μ_{glucose}/μmax_{lactose} ratio were significantly lower and below 0.25. This indicated that at least the scrA₈₂₉^{FS} mutation had negative impact on the growth of S. thermophilus on glucose. The growth on glucose is not completely abolished but significantly slower; this phenotype was defined as “glucose slow-growing”.

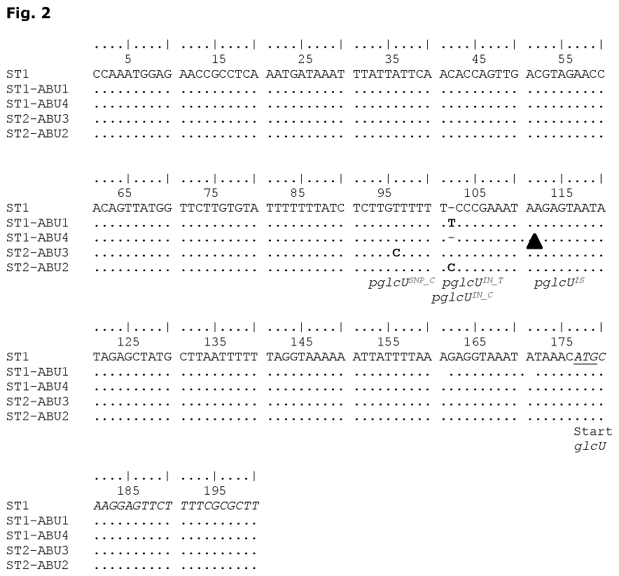

Spontaneous mutants displaying an improved ability to consume glucose could possibly arose from the glucose slow-growing strains upon cultivation on glucose as a sole source of carbohydrate (possibly explaining the high OD₆₀₀ recorded upon their cultivation for 24 hours in M17-glucose medium, not shown). Cultures of several scrA₈₂₉^{FS}-pscrB₈₂₉ strains were plated on M17-agar plates containing 30 g/L of glucose as a sole source of carbohydrate. Colonies were picked upon cultivation at 37° C. and their ability to grow on glucose was investigated as described in Test B. Some selected clones (ST1-ABU1, ST1-ABU4, ST2-ABU1, ST2-ABU2, ST2-ABU3, ST3-ABU1 and ST3-ABU2) displayed a μ_{glucose}/μmax_{lactose} ratio in Test B with value similar or higher than that calculated for the ST1, ST2 and ST3 parental strains (Table 3). These clones were further investigated for mutations in their genome. Comparative genomics of the selected clones to their parents allowed to identify 4 different mutations each likely to be responsible for a reversion of the glucose slow-growing phenotype (SEQ ID NO: 217-220). Mutations were found for every clone located at the genetic locus corresponding to the promoter region upstream of the glcU gene (pglcU); this glcU gene being predicted to code for a non-PTS transporter of glucose. One mutation was a SNP (pglcU^{SNP_C}), two mutations were insertions (pglcU^{IN_T} and pglcU^{IN_C}) of a single nucleotide (same position but different nucleotide), and the last one (pglcU^{IS}) was an insertion of an IS (See FIG. 2).

In order to confirm the mutation in pglcU as responsible for the reversion of the glucose-slow phenotype, ST2-AB was genetically engineered through the insertion of the pglcU^{SNP_C} mutation in the promoter region of glcU to generate strain ST2-ABU3_{ge}. ST2-ABU3_{ge} was compared to ST2-ABU3 in Test B. Growth rate in glucose containing medium of the 2 strains were fast (0.477 and 0.480, respectively) and similar, thus confirming that pglcU^{SNP_C} mutation was responsible for the reversion of the glucose slow-growing phenotype. With the same objective in mind, reverse genetic was performed on ST2-ABU3 to replace the pglcU^{SNP_C} mutation by the wild-type allele of pglcU from the parental ST2. ST2-ABU_{wt} was obtained and compared to ST2-AB in Test B. Growth rate in glucose containing medium of the 2 strains were slow (0.161 and 0.081, respectively) and similar, thus confirming again that pglcU^{SNP_C} mutation is responsible for the reversion of the glucose slow-phenotype phenotype.

TABLE 3
Analysis of the impact of pglcU mutations on the ability of S. thermophilus mutants
bearing the scrA₈₂₉^{FS} mutation with or without the pscrB₈₂₉ mutation to grow on
glucose as a sole source of carbohydrate.

| | | Cell count ratio | | | Ratio μ_{glucose}/ |
|---|---|---|---|---|---|
| Strain | Engineered mutation | sucrose/lactose^{a} | μmax_{lactose}^{b} | μ_{glucose}^{b} | μmax_{lactose}^{b} |
| ST1 | | 0.80 | 0.800 | 0.472 | 0.590 |
| ST1-A | scrA₈₂₉^{FS} | 0.8 10⁻⁸ | 0.745 | 0.044 | 0.059 |
| ST1-AB | scrA₈₂₉^{FS}, pscrB₈₂₉ | <0.5 10⁻⁸ | 0.785 | 0.072 | 0.092 |
| ST1-ABU1 | scrA₈₂₉^{FS}, pscrB₈₂₉, pglcU^{IN}^{_{—}}^{T} | <0.5 10⁻⁸ | 0.790 | 0.683 | 0.865 |
| ST1-ABU4 | scrA₈₂₉^{FS}, pscrB₈₂₉, pglcU^{IS} | <0.5 10⁻⁸ | 0.790 | 0.578 | 0.732 |
| ST2 | | 1 | 0.750 | 0.403 | 0.538 |
| ST2-A | scrA₈₂₉^{FS} | 5.6 10⁻⁸ | 0.740 | 0.076 | 0.103 |
| ST2-AB | scrA₈₂₉^{FS}, pscrB₈₂₉ | <0.5 10⁻⁸ | 0.710 | 0.081 | 0.114 |
| ST2-ABU_{wt} | scrA₈₂₉^{FS}, pscrB₈₂₉ | <0.5 10⁻⁸ | 0.707 | 0.116 | 0.164 |
| ST2-ABU1 | scrA₈₂₉^{FS} pscrB₈₂₉, pglcU^{IN}^{_{—}}^{T} | <0.5 10⁻⁸ | 0.735 | 0.673 | 0.916 |
| ST2-ABU2 | scrA₈₂₉^{FS} pscrB₈₂₉, pglcU^{IN}^{_{—}}^{C} | <0.5 10⁻⁸ | 0.750 | 0.706 | 0.941 |
| ST2-ABU3 | scrA₈₂₉^{FS} pscrB₈₂₉, pglcU^{SNP}^{_{—}}^{C} | <0.5 10⁻⁸ | 0.740 | 0.480 | 0.649 |
| ST2-ABU3_{ge} | scrA₈₂₉^{FS}, pscrB₈₂₉, pglcU^{SNP}^{_{—}}^{C} | <0.5 10⁻⁸ | 0.703 | 0.477 | 0.679 |
| ST3 | | 0.54 | 0.490 | 0.373 | 0.761 |
| ST3-A | scrA₈₂₉^{FS} | <0.5 10⁻⁸ | 0.515 | 0.065 | 0.126 |
| ST3-AB | scrA₈₂₉^{FS}, pscrB₈₂₉ | <0.5 10⁻⁸ | 0.535 | 0.054 | 0.101 |
| ST3-ABU1 | scrA₈₂₉^{FS}, pscrB₈₂₉, pg/cU^{IN}^{_{—}}^{T} | <0.5 10⁻⁸ | 0.485 | 0.488 | 1.010 |
| ST3-ABU2 | scrA₈₂₉^{FS}, pscrB₈₂₉, pg/cU^{IN}^{_{—}}^{C} | <0.5 10⁻⁸ | 0.528 | 0.496 | 0.941 |

^{a}cell counts ratio were established as described in Test A;
^{b}Growth on lactose and glucose over 24 hours were performed as in Test B.

### Example 5: pH_{STOP} of Sweet Fresh-Fermented Products Using S. thermophilus Strains Bearing scrA₈₂₉^{FS} and pscrB₈₂₉ Mutations (with or without a pglcU Mutation) in Addition to Either the Combination of glcK_{E275K} and manM₁₂₉₉₇ Mutations or the Combination of ccpA₈₅₅ and manM₁₂₉₉₇ Mutations)

Lactose-positive, galactose-negative, Streptococcus thermophilus strains carrying a mutation in glcK gene and/or in ccpA gene combined with a mutation in one of the genes encoding a protein of the mannose-glucose-specific PTS were shown to be of interest to control end-pH in fresh fermented products like yoghurts (WO2019197051). However, this property was much affected for fresh fermented products in which sucrose was added in order to obtain sweet products.

In order to evaluate the impact and the benefit of the scrA₈₂₉^{FS} and pscrB₈₂₉ mutations as well as of the pglcU mutation in S. thermophilus genome on the acidification kinetic of plain and sweet milk, the following strains were engineered:
ST1-GM-AB, a derivative of ST1-GM (glCK₃₇₈-manM₁₂₉₉₇ double mutant of ST1) bearing the scrA₈₂₉^{FS} and pscrB₈₂₉ mutations
ST1-GM-ABU1, a derivative of ST1-ABU1 bearing the glcK₃₇₈ and manM₁₂₉₉₇ mutations
ST1-CM-ABU1, a derivative of ST1-ABU1 bearing the ccpA₈₅₅ and manM₁₂₉₉₇ mutations

The engineered strains and appropriate control strains (ST1 and ST1-GM) were used to ferment plain milk and sweet milk as follows: fresh pre-culture (in reconstituted milk containing 10% (w/v) skim milk powder pasteurized 20 min at 120° C.) of the strains to be tested was used to inoculate at 2% (v/v) 100 ml of plain milk (“Candia Grand Lait” pasteurized 10 min at 90° C. diluted with water at 93% (v/v)) and 100 mL of sweet milk (“Candia Grand Lait” pasteurized 10 min at 90° C. supplemented with sucrose at 7% (w/v)). The inoculated milks were statically incubated in a water-bath at 43° C. for 24 h. The acidifying properties of the engineered strains were evaluated by recording the pH during milk fermentation. The pH was monitored for 24 hours using the CINAC system (Alliance Instruments, France; pH electrode Mettler 405 DPAS SC, Toledo, Spain). The pH was recorded every 5 or 25 minutes.

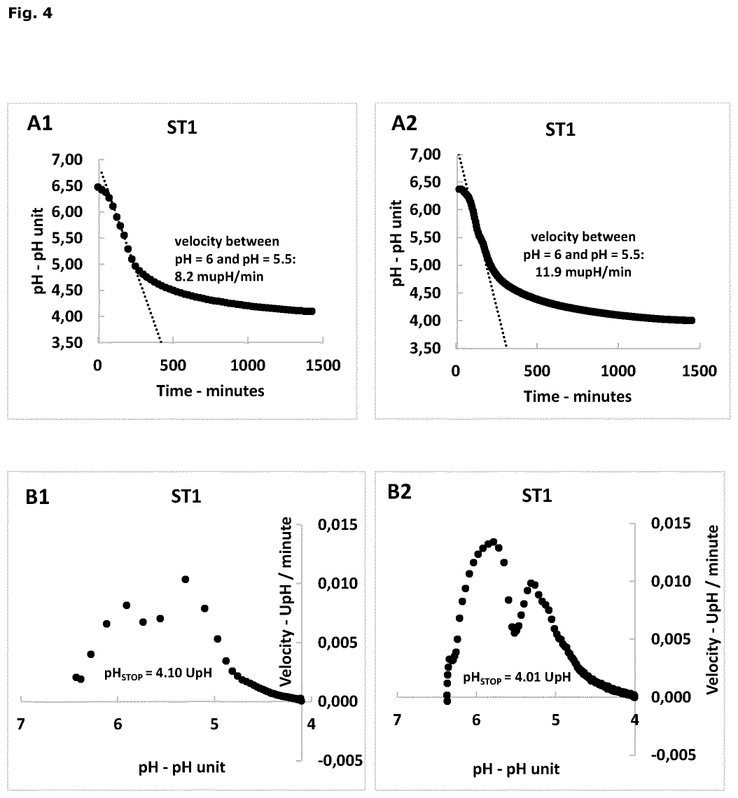

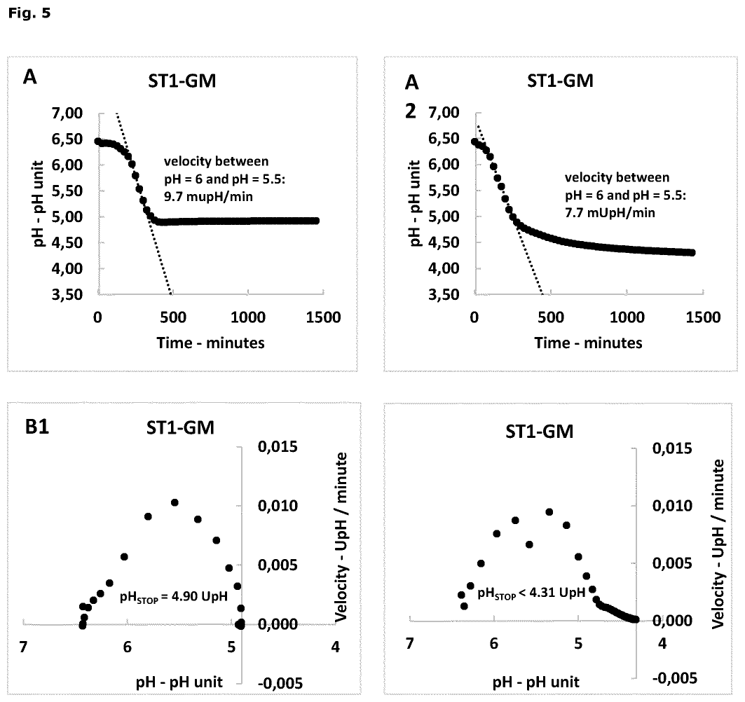

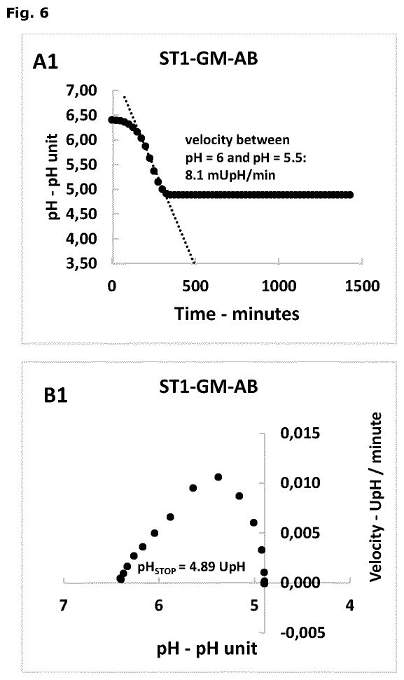

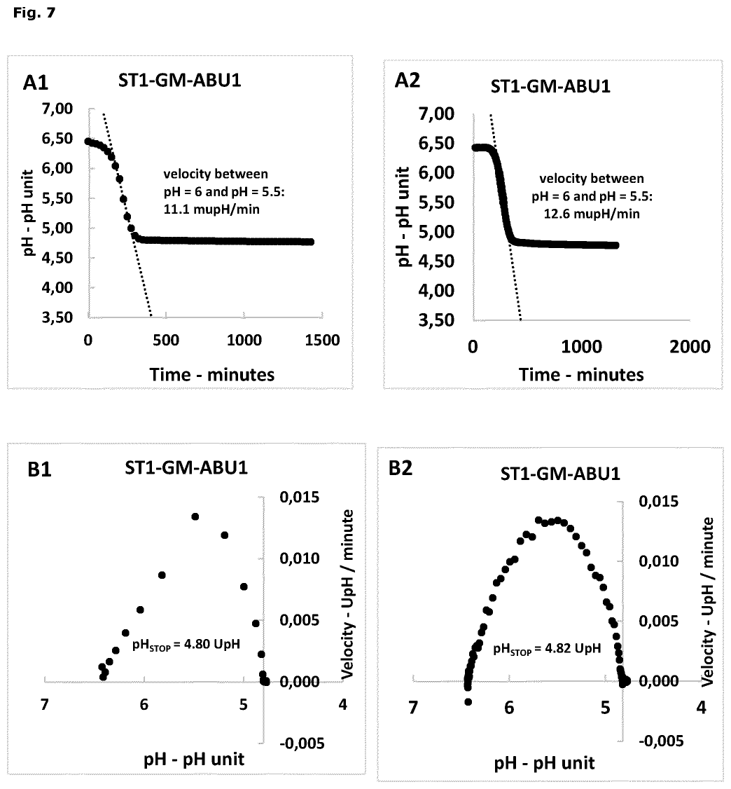

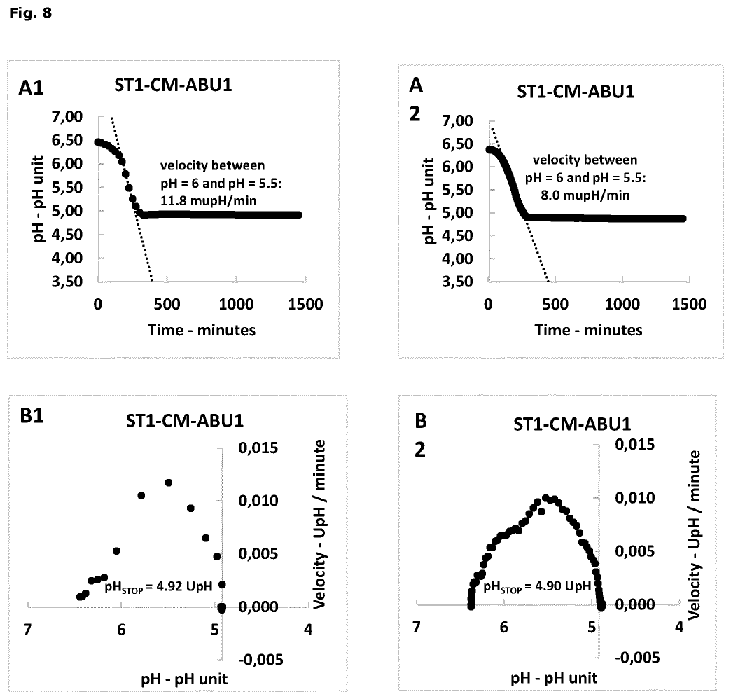

The evolution of the pH over time is represented in FIGS. 4A1, 4A2, 5A1, 5A2, 6A1, 6A2, 7A1, 7A2, 8A1 and 8A2. The velocity between pH 6 and pH 5.5 was calculated as the slope of the linear model deduced from the evolution of the pH as a function of time for value of pH between 6 and 5.5. The slope value is the opposite of the velocity (Table 4). The evolution of the velocity as a function of the pH was also represented (FIGS. 4B1, 4B2, 5B1, 5B2, 6B1, 6B2, 7B1, 7B2, 8B1 and 8B2). Velocity is determined as the instantaneous derivative of the pH evolution as a function of time. The pH_{STOP} was characterized as the pH value at which the speed decrease became non-detectable (below 0.1 mUpH/minute) (Table 4). The time corresponding to the pH_{STOP} (TpH_{STOP}) was also determined (Table 4).

The results are displayed in Table 4. The parental strain (ST1) displayed pH_{STOP} values in the above described condition that were below 4.60 both in plain milk and in sweet milk. As expected, the pH_{STOP} value in plain milk fermented with the glcK₃₇₈-ManM₁₂₉₉₇ double mutant (ST1-GM) was above 4.6. On the contrary, the pH_{STOP} value in the sweet milk was below 4.6, as for the parental strain. On the opposite, the engineered strain ST1-GM-AB bearing the scrA₈₂₉^{FS} and pscrB₈₂₉ mutations displayed high pH_{STOP} value above 4.6 in sweet milk. The presence or not of the pglcU mutation appeared to have no impact on the pH_{STOP} value in plain and sweet milks since it was still above 4.6 for ST1-GM-ABU1. Similarly, a strain bearing the scrA₈₂₉^{FS}, pscrB₈₂₉ and pglcU mutations in addition to the ccpA₈₅₅ and manM₁₂₉₉₇ mutations (ST1-CM-ABU1) displayed high pH_{STOP} values above 4.6 both in plain and sweet fermented milks.

Table 4 indicated as well that the TpH_{STOP} value was reached within 450 minutes in plain milk fermented with the glcK₃₇₈-ManM₁₂₉₉₇ double mutant. However, this double mutant was still acidifiyng sweet milk upon 24 hours of incubation; the TpH_{STOP} value was above 1425 minutes. On the contrary, for strains bearing the scrA₈₂₉^{FS} and pscrB₈₂₉ mutations with or without the pglcU mutation the TpH_{STOP} values were short (below 450 minutes) in both plain and sweet fermented milks. These data also show that neither the addition of the scrA₈₂₉^{FS} and pscrB₈₂₉ mutations nor the pglcU mutation did negatively impact the acidification kinetics (slope between pH 6 and 5.5) which remained acceptable at the industrial level to manufacture fermented dairy products.

Conclusively, the scrA₈₂₉^{FS} and pscrB₈₂₉ as well as pglcU mutations have no impact on the ability of glcK 378-ManM₁₂₉₉₇ and ccpA₈₅₅-ManM₁₂₉₉₇ double mutants to control the end-pH in plain fermented milk. Advantageously, the scrA₈₂₉^{FS} and pscrB₈₂₉ mutations allow to expand this property in milks into which sucrose was added.

TABLE 4
Analysis of the impact of scrA₈₂₉^{FS}, pscrB₈₂₉ and pglcU mutations on the physiological
properties in milk and sweet milk of glcK₃₇₈, ManM₁₂₉₉₇ and ccpA₈₅₅ mutants.

| | | Plain milk^{a} | | | Sweet milk^{b} | | |
|---|---|---|---|---|---|---|---|
| | | | Slope pH | | | Slope pH | |
| | | | 6-5.5 | TpH_{STOP} | | 6-5.5 | TpH_{STOP} |
| Strain | Engineered mutations | pH_{STOP} | (UpH/min) | (min) | pH_{STOP} | (UpH/min) | (min) |
| ST1 | | 4.10 | −0.0082 | 1425 | 4.01 | −0.011.9 | 1335 |
| ST1-GM | glcK₃₇₈, ManM₁₂₉₉₇ | 4.90 | −0.0097 | 450 | <4.31 | −0.0077 | >1425 |
| ST1-GM-AB | glcK₃₇₈, ManM₁₂₉₉₇, | nd | nd | nd | 4.89 | −0.0081 | 400 |
| | scrA₈₂₉^{FS}, pscrB₈₂₉ | | | | | | |
| ST1-GM- | glcK₃₇₈, ManM₁₂₉₉₇, | 4.80 | −0.0111 | 400 | 4.82 | −0.0126 | 440 |
| ABU1 | scrA₈₂₉^{FS}, pscrB₈₂₉, pgl- | | | | | | |
| | cU^{IN}^{_{—}}^{T} | | | | | | |
| ST1-CM- | ccpA₈₅₅, ManM₁₂₉₉₇, | 4.92 | −0.0118 | 350 | 4.90 | −0.0080 | 325 |
| ABU1 | scrA₈₂₉^{FS}, pscrB₈₂₉, pgl- | | | | | | |
| | cU^{IN}^{_{—}}^{T} | | | | | | |

^{a}“Candia Grand Lait” pasteurized 10 min at 90° C. diluted with water at 93% (v/v);
^{b}“Candia Grand Lait” pasteurized 10 min at 90° C. supplemented with sucrose at 7% (w/v);
nd, not determined.

### Example 6. Improving Glucose Release and Lactose Consumption Using S. thermophilus Bearing scrA₈₂₉^{FS} and pscrB₈₂₉ Mutations (with or without a pglcU Mutation) in Addition to Either the Combination of glcK_{E275K} and manM₁₂₉₉₇ Mutations or the Combination of ccpA₈₅₅ and manM₁₂₉₉₇ Mutations

In addition to the control the end-pH of fermented milks (see example 5), lactose-positive, galactose-negative S. thermophilus strains carrying a mutation in glcK gene and/or in ccpA gene combined with a mutation in one of the genes encoding a protein of the mannose-glucose-specific PTS were shown to be of interest to overconsume lactose and to release glucose (WO2019197051).

In order to evaluate the impact of the scrA₈₂₉^{FS} and pscrB₈₂₉ mutations as well as of the pglcU mutation on lactose overconsumption and on glucose release, the engineered strains ST1-GM-AB, ST1-GM-ABU1 and ST1-CM-ABU1) described in example 5 were further investigated. These strains and appropriate control strains (ST1-GM and ST1) were used to ferment sweet milk as described in Example 5. Samples were taken prior to incubation and upon 24 hours of incubation. Biochemical analyses were performed as follows: 5 g of each sample were diluted in 25 g 0.025 N H₂SO₄, before being centrifuged at 4600 rpm for 10 minutes at 4° C. The supernatant was filtered through a 0.2 μm Nylon filter (Phenomenex, Germany, Aschaffenburg) directly into a 2 ml HPLC vial. Glucose and lactose were quantified by high performance liquid chromatography (Agilent 1200 HPLC) equipped with a refractive index detector using an Aminex HPX-87H anion exchange column (Bio-Rad Laboratories Inc.) at 35° C., with 12.5 mM H₂SO₄ as the elution fluid and a flow rate of 0.6 ml min-1. The exploitation of the results was made with Chemstation reprocessing software (Agilent).

The results are displayed in Table 5. The parental strain (ST1) consumed a limited amount of lactose (less than 50% considering that the initial concentration in sweet milk was 145 mM) and no measurable amount of glucose was detected from the hydrolysis of the carbohydrates. As expected, and as shown previously (WO2019197051), a double-mutant bearing both the glcK₃₇₈ and ManM₁₂₉₉₇ mutations (ST1-GM) was able to consume more lactose (only 44 mM remaining) and to release up to 44 mM glucose. Introducing the scrA₈₂₉^{FS} and pscrB₈₂₉ mutations into the genome of ST1-GM (generating ST1-GM-AB) resulted in a strain that was consuming even more lactose (16 mM remaining lactose) with the consequence of releasing more glucose (71 mM). Finally, the addition of a mutation in the promoter region of glcU of the genome of ST1-GM-AB was resulting in a strain (ST1-GM-ABU1) capable of hydrolyzing the totality of the lactose present in milk upon 24 hours of fermentation and more surprisingly (considering that the pglcU mutation improved the growth on glucose of scrA₈₂₉^{FS}-pscrB₈₂₉ mutants) to release even more glucose up to 91 mM. Similar behavior was observed with a strain bearing the ccpA₈₅₅ mutation instead of the glcK₃₇₈ mutation (strain ST1-CM-ABU1).

Conclusively, the scrA₈₂₉^{FS} and pscrB₈₂₉ mutations as well as the pglcU mutation have no negative impact on the phenotype (overconsumption of lactose and release of glucose) conferred by the glcK₃₇₈ and ManM₁₂₉₉₇ or ccpA₈₅₅ and ManM₁₂₉₉₇ mutations in milk fermentation. On the contrary, these mutations allow to obtain strains that hydrolyze even more lactose and that release even more glucose.

TABLE 5
Analysis of the impact of scrA₈₂₉^{FS}, pscrB₈₂₉and pglcU mutations
on the ability of glcK₃₇₈-ManM₁₂₉₉₇ and ccpA₈₅₅-ManM₁₂₉₉₇ mutants
to consume lactose and to release glucose upon milk fermentation.

| | | Remaining | Released |
|---|---|---|---|
| Strain | Engineered mutations | Lactose (mM)^{a} | glucose (mM)^{a} |
| ST1 | | 104 | <2 |
| ST1-GM | glck₃₇₈, ManM₁₂₉₉₇ | 44 | 44 |
| ST1-GM-AB | glck₃₇₈, ManM₁₂₉₉₇, scrA₈₂₉^{FS}, pscrB₈₂₉ | 16 | 71 |
| ST1-GM-ABU1 | glck₃₇₈, ManM₁₂₉₉₇, scrA₈₂₉^{FS}, pscrB₈₂₉, pglcU | <1 | 91 |
| ST1-CM-ABU1 | ccpA₈₅₅, ManM₁₂₉₉₇, scrA₈₂₉^{FS}, pscrB₈₂₉, pglcU | <1 | 100 |

^{a}“Candia Grand Lait” pasteurized 10 min at 90° C. supplemented with sucrose at 7% (w/v).

### Example 7. scrA₈₂₉^{FS}-pscrB₈₂₉ Double Mutation Impairs Transport of Sucrose and Glucose into the Cell and pglcU Mutation Restores the Transport of Glucose into the Cell of the Double Mutant

In order to elucidate the mechanism leading to the sucrose-negative and glucose slow-growing phenotypes, experimental investigations were performed on the transport of carbohydrates using resting-cells (non-growing cells) of ST1 and its scrA₈₂₉^{FS}-pscrB₈₂₉ (ST1-AB) and scrA₈₂₉^{FS}-pscrB₈₂₉-pglcU^{IN_T} (ST1-ABU1) mutants.

Strains were pre-cultivated overnight at 37° C. in M17 containing 30 g/L of lactose. The pre-culture was used to seed M17 containing 30 g/L of lactose at a final OD₆₀₀ of 0.05 and incubated for 4 hours at 42° C. Cells were harvested by centrifugation at 2450 g for 7 min, washed twice with 4% (w/v) beta-glycerophosphate buffer then resuspended in 10 mL of beta-glycerophosphate buffer. The OD₆₀₀ of the final cell suspension was recorded. The cell suspension was pre-warmed at 42° C. and glucose, lactose or sucrose was added to a final concentration of 3.5 g/L, 7.0 g/L or 7.0 g/L, respectively. Upon 3 min of incubation at 42° C., the reaction medium was filtered on 0.2 μm nylon membrane (PHENEX GF/NY) and dosage of carbohydrates was performed through high performance liquid chromatography (HPLC Agilent 1100 or 1200) measurement. The samples were injected into an ion exchange column, preceded by its guard column (Security Guard Cartridges, Carbo-Pb 4×3.0 mm ID, Phenomenex). The ion exchange column was Pb2+ (Rezex RPM-Monosaccharide Pb+2, 300×7.8 mm, 8 μm, Phenomenex). The elution was done in isocratic mode with ultra-pure water and the carbohydrates were detected with a refractometer. Quantification was performed by external calibration. Standard solutions were prepared in beta-glycerophosphate 4% (w/v) from a range of 0 to 7 g/L for each carbohydrate. All experiments were duplicated. The sugar consumption speed was calculated in μmol/(u.OD*min.) as follows:

$$\underline{Carbohydrate uptake activity} = [ ( {[ sugar ]}_{T 0} \times 2 / 1.8 \times 1000 / M {W}_{sugar} ) - ( {[ sugar ]}_{T + {3}^{′}} \times 1000 / M {W}_{sugar} ) ] / {OD}_{600} / 3$$

With:
[sugar]_{T0}: initial concentration of carbohydrate (in g/L)
[sugar]_{T+3′}: final concentration of carbohydrate measured after 3 minutes of contact (in g/L)
Volume of cell suspension: 1.8 mL
Volume of cell suspension and sugar solution: 2 mL
MW_{sugar}: molecular weight of the carbohydrate (g/mol)
OD₆₀₀: cell concentration at the beginning of the test

Results are displayed in Table 6. The parental strain ST4 (DSM28255) was capable to efficiently transport lactose, sucrose and glucose with speed of consumption of 0.236, 0.180 and 0.108 μmole/u.OD*min., respectively. These values dramatically decreased for sucrose and glucose (respectively, 0.032 and 0.011 μmole/u.OD*min.) for the scrA₈₂₉^{FS}-pscrB₈₂₉ double mutant (ST4-AB) whereas it was not significantly different for lactose (0.201 μmole/u.OD*min.). Introducing a pglcU^{IN_T} mutation into the genome of the double mutant (ST4-ABU1) had no significant impact on the transport of lactose into the cell (0.227 μmole/u.OD*min.) and did not allow to improve the transport of sucrose (0.004 μmole/u.OD*min.). On the contrary, the pglcU^{IN_T} mutation allowed to fully restore the transport of glucose into the cell (0.102 μmole/u.OD*min.) Conclusively, scrA₈₂₉^{FS}-pscrB₈₂₉ double mutation impairs the transport of sucrose and glucose into the cell and the pglcU^{IN_T} mutation restores the transport of glucose into the cell of the double mutant.

TABLE 6
Transport rate of different carbohydrate by resting cells of
parental ST4 strain and its derivatives ST4-AB and ST4-ABU1.

| | | Sugar transport rate | RSD^{a} |
|---|---|---|---|
| Added sugar | Strain | (μmol/(u · DO*min.)) | (%) |
| Lactose | ST4 | 0.236 | 12 |
| 70 g/L | ST4-AB | 0.201 | 5 |
| | ST4-ABU1 | 0.227 | 12 |
| Sucrose | ST4 | 0.180 | 2 |
| 70 g/L | ST4-AB | 0.032 | 6 |
| | ST4-ABU1 | 0.004 | 403 |
| Glucose | ST4 | 0.108 | 6 |
| 35 g/L | ST4-AB | 0.011 | 11 |
| | ST4-ABU1 | 0.102 | 12 |

^{a}RSD, Relative Standard Deviation

SEQUENCES

SEQ ID NO: 2
MSKKLLGIDLGGTTVKFGILTADGEVQEKWAIETNTFENGSHIVPDIVESLKHRLE-
LYGLTAEDFIGIGMGSPGAVDRENKTVTGAFNLNWAETQEVGSVIEKELGIPFAIDNDANVAALGERWVG
AGANNRNVVFITLGTGVGGGVIADGN-
LIHGVAGAGGEIGHIIVEPDTGFECTCGNKGCLETVASATGIVRVAHHLAEKYEGNSSIKAAVDNGEFVTS
KDIIVAATEGDKFADSIVDKVSKYLGLATANISNILNPDSVVIGGGVSAAGEFLRS-
RVEGYFTRYAFPQVRRTTKVKLAELGNDAGIIGAASLAYSIDK
SEQ ID NO: 22
MSKKLLGIDLGGTTVKFGILTADGEVQEKWAIETNTFENGSHIVPDIVESLKHRLE-
LYGLTAEDFIGIGMGSPGAVDRENKTVTGAFNLNWAETQEVGSVIEKELGIPFAIDNDANVAALGERWVG
AGANNRNVVFITLGTGVGGGVIADGN-
LIHGVAGAGGEIGHIIVEPDTGFECTCGNKGCLETVASATGIVRVAHHLAEKYEGNSSIKAAVDNGEFVTS
KDIIVAATEGDKFADSIVDKVSKYLGLATANISNILNPDSVVIGGGVSAAGKFLRS-
RVEGYFTRYAFPQVRRTTKVKLAELGNDAGIIGAASLAYSIDK
SEQ ID NO: 25 (X underlined in bold corresponds to the aa at position 275 and can be
any naturally occurring amino acid, but not Glu)
MSKKLLGIDLGGTTVKFGILTADGEVQEKWAIETNTFENGSHIVPDIVESLKHRLELYGL
TAEDFIGIGMGSPGAVDRENKTVTGAFNLNWAETQEVGSVIEKELGIPFAIDNDANVAAL
GERWVGAGANNRNVVFITLGTGVGGGVIADGNLIHGVAGAGGEIGHIIVEPDTGFECTCG
NKGCLETVASATGIVRVAHHLAEKYEGNSSIKAAVDNGEFVTSKDIIVAATEGDKFADSI
VDKVSKYLGLATANISNILNPDSVVIGGGVSAAGXFLRSRVEGYFTRYAFPQVRRTTKVK
LAELGNDAGIIGAASLAYSIDK
SEQ ID NO: 45
MSKKLLGIDLGGTTVKFGILTADGEVQEKWAIETNTFENGSHIVPDIVESLKHRLE-
LYGLTAEDFIGIGMGSPGAVDRENKTVTGAFNLNWAETQEVGSVIEKELGIPFAIDNDANVAALGERWVG
AGANNRNVVFITLGTGVSGGVIADGN-
LIHGVAGAGGEIGHIIVEPDTGFECTCGNKGCLETVASATGIVRVAHHLAEKYEGNSSIKAAVDNGEFVTS
KDIIVAATEGDKFADSIVDKVSKYLGLATANISNILNPDSVVIGGGVSAAGEFLRS-
RVEGYFTRYAFPQVRRTTKVKLAELGNDAGIIGAASLAYSIDK
SEQ ID NO: 23
MSKKLLGIDLGGTTVKFGILTADGEVQEKWAIETNTFENGSHIVPDIVESLKHRLE-
LYGLTAEDFIGIGMGPPGAVDRENKTVTGAFNLNWAETQEVGSVIEKELGIPFAIDNDANVAALGERWVG
AGANNRNVVFITLGTGVGGGVIADGN-
LIHGVAGAGGEIGHIIVEPDTGFECTCGNKGCLETVASATGIVRVAHHLAEKYEGNSSIKAAVDNGEFVTS
KDIIVAATEGDKFADSIVDKVSKYLGLATANISNILNPDSVVIGGGVSAAGEFLRS-
RVEGYFTRYAFPQVRRTTKVKLAELGNDAGIIGAASLAYSIDK
SEQ ID NO: 65
ATGAATACTGATGAAACAATCACAATTTATGATGTAGCGCGTGAAGCTGGAGTATCGATGG-
CAACTGTTTCTCGTGTTGTAAATGGTAACAAAAACGTAAAAGAAAACACCCGAAAAAAAGTGCTCGAAG
TCATTGATCGTTTGGATTACCGTCCAAATGCGGTTGCGCGTGGCTTGG-
CAAGTAAAAAAACAACTACTGTAGGAGTTGTCATTCCAAATATTGTAAATAGCTATTTTGCTACTCTAGC
TAAAGGTATTGATGACATTGCAACCATGTATAAGTATAATATTGTTCTTGCTTCCAG-
TGATGATAATGAGGATCATGAAGTTACAGTCATTCATTCTCTAATTTCTAAACAAGTTGATGGTATTATT
TTTATGGGACACCATCTGACAGAAAAAATCCGTGCAGAATTCTCTCGTACCCGTACAC-
CGATTGTTCTAGCAGGAACAGTTGATCTCGAACACCAATTACCAAGTGTTAACATCGACTATAAAGCTG
CCGTTGAGGATTGTGTAACGCAACTTGCTAAAAATAATGAAAAGGTTGCCTTTGTATCAG-
GACCACTAATTGATGATATTAATGGCAAACTACGTTTGGCCGGGTATAAGTCTGGACTTGAAAAGAATA
ATTTGAGCTACAACGAAGGACTTGTCTTTGAAGCTAAATATAGCTATAAAGACGGCTTTGAG-
TTAGCACAACGTGTCTTGAACTCTGGTGCCACTGCTGCCTATGTTGGGGAAGATGAATTGGCTGCAGG
TCTCTTGAATGGCCTCTTTGCTGCAGGCAAATCAGTTCCAGAAGATTTCGAAATCATCACAA-
GCAATGATTCACCGGTTACAAGCTACACACGTCCAAACCTTTCTAGTATAAACCATCCTCTCTATGATTT
AGGGGCAGTTAGCATGCGTATGTTGACTAAAATTATGCATAAGGAAGAACTTGAAGATAAA-
GACGTTATTCTTAATCATGGTCTAACTTTACGCCAGTCAACAAAATAA
SEQ ID NO: 71
ATGAATACTGATGAAACAATCACAATTTATGATGTAGCGCGTGAAGCTGGAGTATCGATGG-
CAACTGTTTCTCGTGTTGTAAATGGTAACAAAAACGTAAAAGAAAACACCCGAAAAAAGTGCTCGAAGT
CATTGATCGTTTGGATTACCGTCCAAATGCGGTTGCGCGTGGCTTGG-
CAAGTAAAAAAACAACTACTGTAGGAGTTGTCATTCCAAATATTGTAAATAGCTATTTTGCTACTCTAGC
TAAAGGTATTGATGACATTGCAACCATGTATAAGTATAATATTGTTCTTGCTTCCAG-
TGATGATAATGAGGATCATGAAGTTACAGTCATTCATTCTCTAATTTCTAAACAAGTTGATGGTATTATT
TTTATGGGACACCATCTGACAGAAAAAATCCGTGCAGAATTCTCTCGTACCCGTACAC-
CGATTGTTCTAGCAGGAACAGTTGATCTCGAACACCAATTACCAAGTGTTAACATCGACTATAAAGCTG
CCGTTGAGGATTGTGTAACGCAACTTGCTAAAAATAATGAAAAGGTTGCCTTTGTATCAG-
GACCACTAATTGATGATATTAATGGCAAACTACGTTTGGCCGGGTATAAGTCTGGACTTGAAAAGAATA
ATTTGAGCTACAACGAAGGACTTGTCTTTGAAGCTAAATATAGCTATAAAGACGGCTTTGAG-
TTAGCACAACGTGTCTTGAACTCTGGTGCCACTGCTGCCTATGTTGGGGAAGATGAATTGGCTGCAGG
TCTCTTGAATGGCCTCTTTGCTGCAGGCAAATCAGTTCCAGAAGATTTCGAAATCATCACAA-
GCAATGATTCACCGGTTACAAGCTACACACGTCCAAACCTTTCTAGTATAAACCATCCTCTCTATGATTT
AGGGGCAGTTAGCATGCGTATGTTGACTAAAATTATGCATAAGGAAGAACTTGAAGATAAA-
GACGTTATTCTTAATCATGGTCTAACTTTACGCCAGTCAACAAAATAA
SEQ ID NO: 78
MGIGIIIASHGRFAEGIHQSGSMIFGDQEKVQVVTFMPSEGPDDLYAHFNNAIA-
QFDVDDEILVLADLWSGSPFNQASRIARENPDRKIAIITGLNLPMLIQAYTERMMDANATVEQVAANIIKEA
KGGIKALPEELNPAEETTAAPVEAAAPQGAIPEGTVIGDGKLKINLARLD-
TRLLHGQVVTNWVPYSKADRIIVASDDVAKDELRKELIKQAAPNGIKVNVVPIQKLIDASKDPRFGNTHAL
VLFETVQDALRAIEGGVPIKELNVGSMAHSTGKTMVNNVLS-
MDKDDVACFEKLRDLGVEFDVRKVPNDSKKDLFELIKKANVQ
SEQ ID NO: 111
ATGGGTATCGGTATTATTATTGCCAGCCATGGTAGGTTCGCTGAAGGAATCCAC-
CAATCAGGCTCTATGATTTTTGGGGACCAAGAGAAAGTTCAAGTTGTGACTTTCATGCCAAGTGAAGGT
CCTGATGATTTGTACGCTCACTTCAACAACGCCATTGCACAATTCGATGTT-
GATGATGAAATTCTTGTTTTGGCTGACCTTTGGAGTGGTTCACCATTTAACCAAGCTAGTCGAATCGCT
AGGGAAAATCCAGATCGCAAGATTGCTATCATCACAGGACTTAACTT-
GCCAATGCTAATCCAAGCATACACTGAACGTATGATGGATGCTAACGCTACTGTAGAGCAAGTTGCTG
CTAATATCATCAAGGAAGCTAAGGGTGGTATCAAGGCACTTCCAGAA-
GAGCTAAATCCAGCTGAGGAAACAACTGCAGCTCCTGTAGAAGCTGCAGCACCTCAAGGAGCTATCCC
TGAAGGAACAGTCATCGGAGATGGTAAACTCAAGATTAACTTGGCACGTTTGGACACAC-
GTCTCTTGCATGGTCAAGTAGTAACTAACTGGGTACCTTATTCTAAAGCAGACCGTATTATTGTTGCTTC
GGATGACGTTGCCAAAGATGAGCTTCGTAAGGAATTGATCAAACAGGCTGCACCAAACGG-
TATTAAAGTAAACGTTGTTCCGATTCAAAAATTAATTGATGCTTCTAAAGACCCACGTTTTGGAAATACA
CATGCGCTTGTCTTGTTCGAAACTGTTCAAGACGCACTTCGTGC-
TATCGAAGGTGGCGTGCCAATAAAAGAACTTAACGTTGGTTCTATGGCTCACTCAACTGGTAAAACAAT
GGTTAACAACGTTTTGTCTATGGATAAAGATGATGTTGCTTGTTTTGAAAAATTACGTGAC-
CTTGGCGTTTAATTTGACGTCCGTAAGGTTCCAAACGATTCTAAGAAAGATTTGTTTGAGCTTATCAAG
AAAGCTAACGTTCAATAA
SEQ ID NO: 112
MGIGIIIASHGRFAEGIHQSGSMIFGDQEKVQVVTFMPSEGPDDLYAHFNNAIA-
QFDVDDEILVLADLWSGSPFNQASRIARENPDRKIAIITGLNLPMLIQAYTERMMDANATVEQVAANIIKEA
KGGIKALPEELNPAEETTAAPVEAAAPQGAIPEGTVIGDGKLKINLARLD-
TRLLHGQVVTNWVPYSKADRIIVASDDVAKDELRKELIKQAAPNGIKVNVVPIQKLIDASKDPRFGNTHAL
VLFETVQDALRAIEGGVPIKELNVGSMAHSTGKTMVNNVLSMDKDDVACFEKLRDLGV
SEQ ID NO: 130
MSDMSIISAILVVAVAFLAGLESILDQFQFHQPLVACTLIGAATGNLTAGIM-
LGGSLQMITLAWANIGAAVAPDVALASVAAAIILVKGGKFTAEGIGVAIAIAILLAVAGLFLTMPVRTASIAF
VHAADKAAEHGNIAGVERAYYLALLLQGLRIAVPAALLLAIPAQSVQHALGLMP-
DWLTHGLVVGGGMVVAVGYAMIINMMATREVWPFFAIGFALAAISQLTLIALSTIGVAIAFIYLNLSKQGG
GNGGGNGGGTSSGSGDPIGDILEDY
SEQ ID NO: 157
ATGTCAGATATGTCAATTATTTCTGCGATTTTGGTCGTAGCTGTTGCCTTCCTT-
GCTGGTCTTGAAAGTATCCTTGACCAATTCCAATTCCACCAACCACTTGTTGCATGTACCCTCATCGGT
GCTGCCACAGGTAACCTCACTGCAGGTATCATGCTTGGTGGTTCTCTTCAAATGAT-
TACCCTTGCTTGGGCAAACATCGGTGCTGCCGTAGCTCCTGACGTTGCCCTTGCATCTGTTGCCGCTG
CCATCATTTTGGTTAAAGGTGGTAAATTTACAGCTGAAGGTATCGGTGTTGCGATTGCAA-
TAGCTATCCTGCTTGCAGTTGCAGGTCTCTTCCTAACTATGCCTGTTCGTACAGCATCTATTGCCTTTGT
TCATGCTGCAGATAAAGCTGCAGAACACGGAAACATCGCTGGTGTTGAACGTGCATACTAC-
CTCGCTCTCCTTCTTCAAGGTTTGCGTATTGCTGTGCCAGCAGCCCTTCTTCTTGCCATCCCGGCCCAA
TCTGTTCAACATGCCCTTGGCTTGATGCCTGACTGGCTCACCCATGGTTTGGTT-
GTCGGTGGTGGTATGGTCGTAGCCGTTGGTTACGCCATGATTATCAATATGATGGCTACTCGTTAAGTT
TGGCCATTCTTCGCCATTGGTTTTGCTTTGGCAGCAATTAGCCAATTGACAC-
TTATCGCTCTTAGTACCATTGGTGTTGCCATCGCCTTCATCTACCTCAACCTTTCTAAACAAGGTGGCG
GAAATGGTGGCGGAAATGGTGGCGGAACTTCATCTGGTTCAGGCGACCCAATCGGCGA-
TATCTTGGAAGACTACTAG
SEQ ID NO: 158
MSDMSIISAILVVAVAFLAGLESILDQFQFHQPLVACTLIGAATGNLTAGIM-
LGGSLQMITLAWANIGAAVAPDVALASVAAAIILVKGGKFTAEGIGVAIAIAILLAVAGLFLTMPVRTASIAF
VHAADKAAEHGNIAGVERAYYLALLLQGLRIAVPAALLLAIPAQSVQHALGLMP-
DWLTHGLVVGGGMVVAVGYAMIINMMATR
SEQ ID NO: 166
MAEKIQLSQADRKKVWWRSQFLQGAWNYERMQNLGWAYSLIPAIK-
KLYTNKEDQAAALKRHLEFFNTHPYVAAPIIGVTLALEEEKANGTEIEDAAIQGVKIGMMGPLAGIGDPVFW
FTIRPILGALGASLAQAGNIAGPLIFFIGWNLIRMAFLWYTQELGYKAGSEITKDIS-
GGILKDITKGASILGMFILAVLVERWVSVVFTVKLPGKVLPKGAYIEWPKGYVTGDQLKTILGQVNDKLSFD
KIQVDTLQKQLDSLIPGLTGLLLTFACMWLLKKKVSPITIIIGLFVVGIVASFF
GIM
SEQ ID NO: 206
ATGGCTGAAAAAATTCAATTATCTCAAGCGGATCGTAAAAAAGGTTT-
GGTGGCGCTCACAATTCTTGCAAGGTGCATGGAACTATGAACGTATGCAAAACTTGGGTTGGGCTTAC
TCACTCATTCCTGCTATCAAAAAACTTTATACTAACAAAGAGGACCAAGCCG-
CAGCTCTTAAACGTCACTTGGAATTCTTCAACACTCACCCTTACGTAGCTGCTCCTATCATAGGGGTTA
CCTTAGCTCTTGAAGAAGAAAAAGCTAATGGTACTGAAATCGAAGATGCGGC-
TATCCAAGGGGTTAAAATCGGTATGATGGGTCCACTTGCCGGTATCGGTGACCCTGTCTTCTGGTTCAC
AATTCGTCCAATTCTTGGTGCCCTTGGTGCATCATTGGCACAAGCTGGTAACATT-
GCTGGTCCACTTATCTTCTTCATTGGTTGGAACCTTATCCGCATGGCCTTCTTGTGGTACACTCAAGAA
CTTGGTTACAAAGCAGGTTCAGAAATCACTAAAGACATATCTGGTGGTATCTTGAAAGA-
TATTACTAAAGGGGCATCAATACTTGGTATGTTCATCTTGGCCGTCCTCGTTGAACGTTGGGTATCTGT
CGTCTTCACTGTAAAGCTTCCAGGTAAAGTTTTGCCTAAAGGTGCTTA-
TATTGAATGGCCAAAAGGATATGTTACTGGTGACCAACTAAAAACTATCCTTGGTCAAGTCAACGATAA
GCTTAGCTTTGATAAGATTCAAGTCGATACCCTACAAAAACAATTGGAT-
TCATTAATTCCAGGTTTGACGGGACTTCTCCTTACTTTTGCATGTATGTGGTTGCTTAAGAAGAAAGTTT
CACCAATCACAATCATCATCGGACTCTTTGTAGTTGGTATTGTTGCAAGCTTCTTCGGAATCATGTAA
SEQ ID NO: 207
MAEKIQLSQADRKKGLVALTILARCMEL
SEQ ID NO: 208
MAEKIQLSQADRKKGLVALTILARFMEL
SEQ ID NO: 209
MSEKIQLSQADRKKGLVALTILARFMEL
SEQ ID NO: 210
MAEKIQLSQADRRKGLVALTILARFMEL
SEQ ID NO: 211
MSEKIQLSQADHKKGLVALTILARFMEL
SEQ ID NO: 212_partial scrA829
ATGGATTACAAACAAATTGCAAAAGAAGTCATCGAAGCCCTCGGTGGAC-
GTGAAAATGTTAACAGTGTTGCTCACTGTGCGACACGTCTACGTGTTATGGTTAAAGATGAAAACAAAA
TCAATAAAGAAAAAAGCTGAGAATATTGAAAAAGTTCAAGG
SEQ ID NO: 213_pscrB829
ATTTAATAATCTCCTAATTTATTTATTAGTAATATAGGAAACGTTTTACCTA-
GAAAAATCAACGATTTATAATAAGAAAACAAAAAAATGCTAAACGTTTGACATATGACAGAAAGATGTT
AAAATTAATATCGTAAAGAAAAGTGAAACGTTTTCAAAAACAAATTTTGTTAAA-
GATGTTAAAATTGATATCGTAAAGAAAAGCGAAACGTTTTCAAAAACAAATTTTATTAAGGAGAATTTTG
CAA
SEQ ID NO: 214
ATGGATTACAAACAAATTGCAAAAGAAGTCATCGAAGCCCTCGGTGGAC-
GTGAAAATGTTAACAGTGTTGCTCACTGTGCGACACGTCTACGTGTTATGGTTAAAGATGAAAACAAAA
TCAATAAAGAAAAAGCTGAGAATATTGAAAAAGTTCAAGG
SEQ ID NO: 215
ATTTAATAATCTCCTAATTTATTTATTAGTAATATAGTAAACGTTTTACCTA-
GAAAAATCAACGATTTATAATAAGAAAACAAAAAAATGCTAAACGTTTGACATATGACAGAAAGATGTT
AAAATTAATATCGTAAAGAAAAGTGAAACGTTTTCAAAAACAAATTTTGTTAAA-
GATGTTAAAATTGATATCGTAAAGAAAAGCGAAACGTTTTCAAAAACAAATTTTATTAAGGAGAATTTTG
CAA
SEQ ID NO: 216
CCAAATGGAGAACCGCCTCAAATGATAAATTTATTATTCAACACCAGTTGACGTAGAAC-
CACAGTTATGGTTCTTGTGTATTTTTTTATCTCTTGTTTTTTCCCGAAATAAGAGTAATATAGAGCTATGC
TTAATTTTTTAGGTAAAAAATTATTTTAAAGAGGTAAATATAAAC
SEQ ID NO: 217
CCAAATGGAGAACCGCCTCAAATGATAAATTTATTATTCAACACCAGTTGACGTAGAAC-
CACAGTTATGGTTCTTGTGTATTTTTTTATCTCTTGTTTTTTTCCCGAAATAAGAGTAATATAGAGCTATG
CTTAATTTTTTAGGTAAAAAATTATTTTAAAGAGGTAAATATAAAC
SEQ ID NO: 218
CCAAATGGAGAACCGCCTCAAATGATAAATTTATTATTCAACACCAGTTGACGTAGAAC-
CACAGTTATGGTTCTTGTGTATTTTTTTATCTCTTGTTTTTTCCCCGAAATAAGAGTAATATAGAGCTATG
CTTAATTTTTTAGGTAAAAAATTATTTTAAAGAGGTAAATATAAAC
SEQ ID NO: 219
CCAAATGGAGAACCGCCTCAAATGATAAATTTATTATTCAACACCAGTTGACGTAGAAC-
CACAGTTATGGTTCTTGTGTATTTTTTTATCTCTTGCTTTTTCCCGAAATAAGAGTAATATAGAGCTATGC
TTAATTTTTTAGGTAAAAAATTATTTTAAAGAGGTAAATATAAAC
SEQ ID NO: 220
CCAAATGGAGAACCGCCTCAAATGATAAATTTATTATTCAACACCAGTTGACGTAGAACC
ACAGTTATGGTTCTTGTGTATTTTTTTATCTCTTGTTTTTTCCCGAAATAGGTAGTGTAA
AATAAGTTGTGTAAACACAAAAAGGAATAAATCCGTTATAGTAGAGTTGCAAAACATTAC
TAGAAAGAGATTTATTCCTATGACTCCGTTTACCACAGAACTACTTAACTTCCTTGCCCA
AAAGCAAGATATTGATGAATTTTTCCGTACTTCTCTTGAAACAGCTATGAATGATCTGCT
TCAAGCAGAGTTATCAGCCTTTTTAGGGTATGAACCTTACGATAAATTAGGCTATAATTC
TGGGAATAGTCGTAACGGAAGCTATGCACGGAAATTCGAAACCAAATATGGGACTGTTCA
GTTGAGTATTCCTAGAGATCGTAATGGGAACTTTAGTCCAGCTTTGCTTCCCGCTTATGG
ACGTCGAGATGACCACTTGGAAGAGATGGTTATCAAACTCTATCAAACCGGTGTAACGAC
TCGAGAAATTAGTGATATCATCGAGCGAATGTATGGTCATCACTATAGTCCTGCCACAAT
TTCTAATATCTCAAAAGCAACTCAGGAGAATGTCGCTACTTTTCATGAGCGAAGCTTAGA
AGCCAATTACTCTGTTTTATTTCTTGACGGAACCTATCTTCCCTTAAGACGTGGAACCGT
TAGTAAAGAATGTATTCATATCGCACTTGGCATTACACCAGAAGGACAGAAGGCTGTTCT
TGGATATGAAATCGCTCCAAATGAAAACAATGCTTCTTGGTCCACCCTGTTAGACAAGCT
TCAAAACCAAGGAATCCAACAGGTTTCTCTTGTAGTGACCGATGGCTTCAAGGGGCTTGA
AGAGATTATCAATCAGGCTTACCCATTAGCTAAACAACAACGTTGCTTAATTCATATTAG
TCGAAATCTAGCTAGTAAAGTGAAACGAGCAGATAGAGCGGTTATTCTGGAGCAATTTAA
AACGATTTATCGTGCTGAAAATTTAGAAATGGCAGTGCAAGCTTTAGAGAACTTTATCGC
CGAATGGAAACCAAAGTATAGGAAAGTCATGGAAAGTCTGGAGAATACGGATAATCTTTT
AACTTTTTATCAGTTTCCCTACCAGATTTGGCATAGCATTTATTCGACAAACCTCATTGA
GTCTCTTAACAAAGAAATCAAACGTCAAACGAAAAAGAAGGTCCTTTTTCCTAACGAGGA
GGCTCTGGAACGTTATTTAGTTACCCTGTTTGAAGATTATAATTTCAAGCAAAATCAACG
CATCCATAAAGGGTTTGGACAATGTACTGACACACTTGAAAGCTTATTTGATTAACATTC
TTCAACTCTACTTGAGTGTTTACACATAATTATTGACAGTATCCCGAAATAAGAGTAATA
TAGAGCTATGCTTAATTTTTTAGGTAAAAAATTATTTTAAAGAGGTAAATATAAAC
SEQ ID NO: 221:
GGTAGTGTAAAATAAGTTGTGTAAACACAAAAAGGAATAAATCCGTTATAGTAGAGTTGC
AAAACATTACTAGAAAGAGATTTATTCCTATGACTCCGTTTACCACAGAACTACTTAACT
TCCTTGCCCAAAAGCAAGATATTGATGAATTTTTCCGTACTTCTCTTGAAACAGCTATGA
ATGATCTGCTTCAAGCAGAGTTATCAGCCTTTTTAGGGTATGAACCTTACGATAAATTAG
GCTATAATTCTGGGAATAGTCGTAACGGAAGCTATGCACGGAAATTCGAAACCAAATATG
GGACTGTTCAGTTGAGTATTCCTAGAGATCGTAATGGGAACTTTAGTCCAGCTTTGCTTC
CCGCTTATGGACGTCGAGATGACCACTTGGAAGAGATGGTTATCAAACTCTATCAAACCG
GTGTAACGACTCGAGAAATTAGTGATATCATCGAGCGAATGTATGGTCATCACTATAGTC
CTGCCACAATTTCTAATATCTCAAAAGCAACTCAGGAGAATGTCGCTACTTTTCATGAGC
GAAGCTTAGAAGCCAATTACTCTGTTTTATTTCTTGACGGAACCTATCTTCCCTTAAGAC
GTGGAACCGTTAGTAAAGAATGTATTCATATCGCACTTGGCATTACACCAGAAGGACAGA
AGGCTGTTCTTGGATATGAAATCGCTCCAAATGAAAACAATGCTTCTTGGTCCACCCTGT
TAGACAAGCTTCAAAACCAAGGAATCCAACAGGTTTCTCTTGTAGTGACCGATGGCTTCA
AGGGGCTTGAAGAGATTATCAATCAGGCTTACCCATTAGCTAAACAACAACGTTGCTTAA
TTCATATTAGTCGAAATCTAGCTAGTAAAGTGAAACGAGCAGATAGAGCGGTTATTCTGG
AGCAATTTAAAACGATTTATCGTGCTGAAAATTTAGAAATGGCAGTGCAAGCTTTAGAGA
ACTTTATCGCCGAATGGAAACCAAAGTATAGGAAAGTCATGGAAAGTCTGGAGAATACGG
ATAATCTTTTAACTTTTTATCAGTTTCCCTACCAGATTTGGCATAGCATTTATTCGACAA
ACCTCATTGAGTCTCTTAACAAAGAAATCAAACGTCAAACGAAAAAGAAGGTCCTTTTTC
CTAACGAGGAGGCTCTGGAACGTTATTTAGTTACCCTGTTTGAAGATTATAATTTCAAGC
AAAATCAACGCATCCATAAAGGGTTTGGACAATGTACTGACACACTTGAAAGCTTATTTG
ATTAACATTCTTCAACTCTACTTGAGTGTTTACACATAATTATTGACAGTATCCCGAAAT
A
SEQ ID NO: 222 >ST12997_manM_DNA_Mutated
ATGTCAGATATGTCAATTATTTCTGCGATTTTGGTCGTAGCTGTTGCCTTCCTTGCTGGT
CTTGAAAGTATCCTTGACCAATTCCAATTCCACCAACCACTTGTTGCATGTACCCTCATC
GGTGCTGCCACAGGTAACCTCACTGCAGGTATCATGCTTGGTGGTTCTCTTCAAATGATT
ACCCTTGCTTGGGCAAACATCGGTGCTGCCGTAGCTCCTGACGTTGCCCTTGCATCTGTT
GCCGCTGCCATCATTTTGGTTAAAGGTGGTAAATTTACAGCTGAAGGTATCGGTGTTGCG
ATTGCAATAGCTATCCTGCTTGCAGTTGCAGGTCTCTTCCTAACTATGCCTGTTCGTACA
GCATCTATTGCCTTTGTTCATGCTGCAGATAAAGCTGCAGAACACGGAAACATCGCTGGT
GTTGAACGTGCATACTACCCTCGCTCTCCTTCTTCAAGGTTTGCGTATTGCTGTGCCAGC
AGCCCTTCTTCTTGCCATCCCGGCCCAATCTGTTCAACATGCCCTTGGCTTGATGCCTGA
CTGGCTCACCCATGGTTTGGTTGTCGGTGGTGGTATGGTCGTAGCCGTTGGTTACGCCAT
GATTATCAATATGATGGCTACTCGTGAAGTTTGGCCATTCTTCGCCATTGGTTTTGCTTT
GGCAGCAATTAGCCAATTGACACTTATCGCTCTTAGTACCATTGGTGTTGCCATCGCCTT
CATCTACCTCAACCTTTCTAAACAAGGTGGCGGAAATGGTGGCGGAAATGGTGGCGGAAC
TTCATCTGGTTCAGGCGACCCAATCGGCGATATCTTGGAAGACTACTAG
SEQ ID NO: 223 >ST12997_manM_Protein_Mutated
MSDMSIISAILVVAVAFLAGLESILDQFQFHQPLVACTLIGAATGNLTAGIMLGGSLQMI
TLAWANIGAAVAPDVALASVAAAIILVKGGKFTAEGIGVAIAIAILLAVAGLFLTMPVRT
ASIAFVHAADKAAEHGNIAGVERAYYPRSPSSRFAYCCASSPSSCHPGPICSTCPWLDA*

### Strains

DGCC numbers are internal references to DuPont Danisco collection; DSM numbers are the numbers assigned by the Leibniz-Institut DSMZ-Deutsche Sammlung von Mikroorganismen und Zellkulturen, GmbH (Inhoffenstr. 7B, D-38124 Braunschweig), following deposit under the Budapest Treaty.

As far as the Streptococcus thermophilus strain DGCC7710 deposited under the Budapest Treaty at the Leibniz-Institut DSMZ-Deutsche Sammlung von Mikroorganismen und Zellkulturen, GmbH, on Jan. 14, 2014 under number DSM28255 is concerned, we hereby confirm that the depositor, Danisco Deutschland GmbH (of Busch-Johannsen-Strasse 1, D-25899 Niebüll, Germany) has authorised the Applicant (DuPont Nutrition Biosciences ApS) to refer to the deposited biological material in this application. The expressions “DGCC7710 strain” and “DGCC7710 derivative” are used interchangeably with the expressions “DSM28255 strain” and “DSM28255 derivative”.

A derivative of the DGCC7710 strain was designed, into which the glcK gene encodes a glucokinase with the glutamic acid (E) at position 275 was replaced by the amino acid lysine (K). This derivative (DGCC12534) was deposited at the DSMZ on Aug. 15, 2017 under accession number DSM32587.

Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany].

The following strains have been deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022:
Streptococcus thermophilus strain ST1-ABU1 (Deposited as DSM34134);
Streptococcus thermophilus strain ST1-ABU4 (Deposited as DSM34140);
Streptococcus thermophilus strain ST1-GM-ABU1 (Deposited as DSM34138);
Streptococcus thermophilus strain ST1-CM-ABU1 (Deposited as DSM34139);
Streptococcus thermophilus strain ST2 (Deposited as DSM34132);
Streptococcus thermophilus strain ST2-ABU2 (Deposited as DSM34147);
Streptococcus thermophilus strain ST2-ABU3 (Deposited as DSM34146).

Streptococcus thermophilus strain deposited under accession number DSM 34172 (herein referred to as ST1 or DSM34172) on Feb. 16, 2022, at the DSMZ [Deutsche Sammlung von Mikroorganismen und Zellkulturen GmbH, Inhoffenstrasse 7B, D-38124 Braunschweig-Germany].

The applicant requests that a sample of the deposited micro-organisms stated herein may only be made available to an expert, until the date on which the patent is granted.

In respect to those designations in which a European Patent is sought, a sample of these deposited microorganisms will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies.

1. A lactose-positive, sucrose-negative, Streptococcus thermophilus strain carrying one or more mutations selected from the group consisting of:
1) one or more mutation in one or more gene of the sucrose regulon; which mutation provides for a significant reduction in the ability to grow on and/or transport sucrose by said strain; and which mutation at least comprises
i) one or more of the scrA gene, and/or the scrB gene and/or the scrR gene, and
ii) optionally a further mutation in the promoter region regulating the expression of the scrA, scrB and/or scrR genes, such as a mutation that protect the strain from reversing from the sucrose negative phenotype; and
2) optionally one or more further mutation affecting the glucose porter, such as affecting glcU and/or its expression, which mutation restores or improves glucose consumption of said strain when grown on glucose as the sole source of carbohydrates.

2. The lactose-positive, sucrose-negative, Streptococcus thermophilus strain according to claim 1, which strain is glucose-positive or glucose slow-growing.

3. The lactose-positive, sucrose-negative, Streptococcus thermophilus strain according to claim 1 or 2, which strain has one or more mutation in the sucrose regulon in one or more of the scrA gene and the scrB gene.

4. The lactose-positive, sucrose-negative, Streptococcus thermophilus strain according to claim 3, which mutation in one or more gene of the sucrose regulon is in the scrA gene effectively disrupting gene function, such as an insertion or deletion resulting in a frameshift of the open reading frame of the gene coding for the EIIABC^{Sucrose} protein leading to the translation of a truncated EIIABC^{Sucrose} protein encoded by the scrA gene, such as a mutation by insertion or deletion of a nucleotide, such as at or around a nucleotide corresponding to position 133 of SEQ ID NO: 212, in the open-reading-frame (ORF) of the scrA gene.

5. The lactose-positive, sucrose-negative, Streptococcus thermophilus strain according to any one of claims 1-4, which strain has a mutation in the promoter region regulating the expression of any one of the scrA, scrB and/or scrR genes.

6. The lactose-positive, sucrose-negative, Streptococcus thermophilus strain according to claim 5, which mutation is a SNP in the promoter region of the scrA and/or scrB genes, such as a SNP in the promoter region of the scrB gene, such as a mutation near the −10 and −35 sequence of the promoter, such as a mutation of T to G corresponding to position 38 of SEQ ID NO: 213 upstream of the scrB promoter, such as a mutation that will comprise the entire sequence identified by SEQ ID NO:213 or a variant sequence being at least about 90%, such as at least about 95% identical herewith (pscrB₈₂₉).

7. The lactose-positive, sucrose-negative, Streptococcus thermophilus strain according to any one of claims 1-6, which strain has a mutation affecting the glucose porter, which mutation restores or improves glucose consumption of said strain when grown on glucose as the sole source of carbohydrates, such as a full recovery of the growth on glucose as compared to that of a lactose-positive and sucrose-positive Streptococcus thermophilus parental strain.

8. The lactose-positive, sucrose-negative, Streptococcus thermophilus strain according to claim 7, which mutation affecting the glucose porter is affecting glcU and/or its expression, such as located within the promoter region of glcU, such as mutations selected from
a) an insertion of a T nucleotide in the poly-T region at position corresponding to position-75 of the glcU promoter identified as SEQ ID NO: 216 (pglcU^{IN_T}), such as a mutation that will contain a T nucleotide at position 102 of SEQ ID NO: 217, such as a mutation comprising the entire sequence identified by SEQ ID NO: 217 or a variant sequence being at least about 90%, such as at least about 95% identical herewith;
b) an insertion of a C nucleotide in the poly-T region at position corresponding to position-75 of the glcU promoter identified as SEQ ID NO: 216 (pglcU^{IN_C}), such as a mutation that will contain a C nucleotide at position 102 of SEQ ID NO: 218, such as a mutation that will comprise the entire sequence identified by SEQ ID NO: 218 or a variant sequence being at least about 90%, such as at least about 95% identical herewith;
c) a mutation of a T to a C nucleotide in the poly-T region at position corresponding to position-81 of the glcU promoter identified as SEQ ID NO: 216 (pglcU^{SNP_C}), such as a mutation that will contain a C nucleotide at position 96 of SEQ ID NO: 219, such as a mutation that will comprise the entire sequence identified by SEQ ID NO:219 or a variant sequence being at least about 90%, such as at least about 95% identical herewith;
d) an insertion of a sequence at a position corresponding to between nucleotide position 110 and 111 of the glcU promoter identified as SEQ ID NO: 216, such as an insertion of a sequence identified by nucleotide 1 to 1321 of SEQ ID NO: 221, such as a mutation that will comprise the entire sequence identified by SEQ ID NO:220 or a variant sequence being at least about 90%, such as at least about 95% identical herewith or SEQ ID NO: 221 or a variant sequence being at least about 90%, such as at least about 95% identical herewith (pglcU^{IS}).

9. The lactose-positive, sucrose-negative, Streptococcus thermophilus strain according to any one of claims 1-8, further comprising one or more mutations selected from:
a) a mutation in the glcK gene encoding a glucokinase, the glucokinase activity of which in said strain is significantly reduced, but not null; and/or
b) a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇); and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167.

10. The lactose-positive, sucrose-negative, Streptococcus thermophilus strain according to any one of claims 1-9, which strain is galactose-negative, such as by mutation in the glcK gene and/or in the ccpA gene optionally combined with a mutation in one of the genes encoding a protein of the man-nose-glucose-specific PTS.

11. The lactose-positive, sucrose-negative, Streptococcus thermophilus strain according to claims 9-10, which strain has a mutation in the glcK gene encoding a glucokinase, which mutation provides for the glucokinase activity in said strain being significantly reduced but not null.

12. The lactose-positive, sucrose-negative, Streptococcus thermophilus strain according to any one of claims 9-11, wherein said mutated glcK gene encodes a glucokinase selected from the group consisting of:
a) a glucokinase having an amino acid at position 275 which is not a glutamic acid, in particular which is not an acidic amino acid, in particular which is a lysine,
b) a glucokinase having an amino acid at position 275 which is not a glutamic acid, in particular which is not an acidic amino acid, in particular which is a lysine (glcK₃₇₈), and having an arginine at position 278 and/or a serine at position 279; and
c) a glucokinase having an amino acid at its position 144 which is not a glycine, in particular which is not an aliphatic amino acid, in particular which is a serine.

13. The lactose-positive, sucrose-negative, Streptococcus thermophilus strain according to any one of claims 9-12, wherein said mutated glcK gene encodes a glucokinase, the sequence of which is selected from the group consisting of:
a) a sequence as defined in SEQ ID NO: 25, wherein the amino acid at position 275 is not a glutamic acid, in particular is not an acidic amino acid, in particular is a lysine; and
b) a Glck variant sequence having at least 90% similarity or identity with SEQ ID NO:25, wherein the amino acid of said glucokinase corresponding to position 275 of SEQ ID NO:25 (or the amino acid at position 275 of said glucokinase) is not a glutamic acid, in particular is not an acidic amino acid, in particular is a lysine;
c) a sequence as defined in SEQ ID NO: 46, wherein the amino acid at position 144 is not a glycine, in particular is not an aliphatic amino acid, in particular is a serine; and
d) a Glck variant sequence having at least 90% similarity or identity with SEQ ID NO: 46, wherein the amino acid of said glucokinase corresponding to position 144 of SEQ ID NO:46 (or the amino acid at position 144 of said glucokinase) is not a glycine, in particular is not an aliphatic amino acid, in particular is a serine.

14. The lactose-positive, sucrose-negative, Streptococcus thermophilus strain according to any one of claims 1-13, further carrying a mutation in the ccpA gene.

15. The lactose-positive, sucrose-negative, Streptococcus thermophilus strain according to any one of claims 1-14, further carrying a mutation in the gene encoding a protein of the mannose-glucose-specific PTS, such as a gene selected from the group consisting of the manL gene, the manM gene and the manN gene.

16. The lactose-positive, sucrose-negative, Streptococcus thermophilus strain according to claim 14 or 15, wherein the ccpA gene carries a mutation selected from the group consisting of a non-sense mutation located between the nucleotide 1 and the nucleotide 270 of the coding sequence of the ccpA gene and a mutation, located in the first quarter of the coding sequence of the ccpA gene, leading to a frameshift of the open reading frame of the ccpA gene.

17. The lactose-positive, sucrose-negative, Streptococcus thermophilus strain according to claim 16, wherein the sequence of said mutated ccpA gene is selected from the group consisting of:
a) a sequence as defined in SEQ ID NO: 71; and
b) a ccpA variant sequence having at least 90%, such as at least 95% identity with SEQ ID NO: 71.

18. The lactose-positive, sucrose-negative, Streptococcus thermophilus strain according to any one of claims 1-17, wherein, when said strain is able to ferment milk as described in Example 5 during 24 hours of fermentation:
a) the concentration of lactose remaining in the fermented milk is less than 60 mM; and/or
b) the pH of the milk decreases to a pH at which the speed of acidification definitively becomes less than 0.1 mUpH/min (pH_{STOP}), wherein said pH_{STOP} is between 4.4 and 5.3, and optionally the slope between pH6 and pH5.5 is at least-0.008 UpH/min.

19. The lactose-positive, sucrose-negative, Streptococcus thermophilus strain according to any one of claims 1-18, wherein said strain has the ability to overconsume lactose as compared to a parental strain without said one or more mutations, such as wherein the remaining amount of lactose when fermenting sweet milk as described and assayed according to Example 6 is lower than 100 mM, such as lower than 90 mM, 80 mM, 70 mM, 60 mM, 50 mM, 40 mM, 30 mM, 20 mM, 10 mM, 8 mM, 6 mM, 4 mM, 2 mM or 1 mM.

20. The lactose-positive, sucrose-negative, Streptococcus thermophilus strain according to any one of claims 1-19, wherein said strain has the ability to release an increased amount of glucose as compared to a parental strain without said one or more mutations, such as wherein the glucose release as described and assayed according to Example 6 is higher than 10 mM, such as higher than 20 mM, 30 mM, 40 mM, 50 mM, 60 mM, 70 mM, 80 mM, or 90 mM.

21. The lactose-positive, sucrose-negative, Streptococcus thermophilus strain according to any one of claims 1-20, wherein said strain is releasing a concentration of glucose assayed as described in example 6, which is at least 50 mM, such as at least 60, 70, 80 or 90 mM or releasing a concentration of glucose which is increased of at least 150% or at least 200%, such as 300%, 400%, 500% as compared to the glucose concentration released by the parent strain, when both assayed as described in example 6.

22. A composition comprising at least one, in particular one, lactose-positive, sucrose-negative, Streptococcus thermophilus strain according to any one of claims 1-21, in particular in combination with another lactic acid bacteria, in particular with one or more strain(s) selected from the group consisting of a strain of the Lactobacillus genus, such as a Lactobacillus delbrueckii subsp bulgaricus strain, a strain of the Lactococcus genus, such as a Lactococcus lactis strain or a strain of the Bifidobacterium genus.

23. A method for manufacturing a fermented dairy product, in particular a fermented milk, comprising inoculating a milk substrate with the lactose-positive, sucrose-negative, Streptococcus thermophilus strain according to any one of claims 1-21, or a composition according to claim 19, and fermenting said inoculated milk, to obtain a fermented dairy product.

24. Use of the lactose-positive, sucrose-negative, Streptococcus thermophilus strain according to any one of claims 1-21, or a composition according to claim 22, to manufacture a fermented dairy product.

25. A fermented dairy product comprising at least one, in particular one, lactose-positive, sucrose-negative, Streptococcus thermophilus strain according to any one of claims 1-21, or as obtained by a method according to claim 23.

26. A Streptococcus thermophilus strain, such as a lactose-positive, sucrose-negative Streptococcus thermophilus strain selected from the group consisting of:
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34132;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34134;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34138;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34139;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34140;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34146;
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 18 Jan. 2022 under number DSM34147; and
a strain deposited under the Budapest Treaty in the name of DuPont Nutrition Biosciences ApS at the Leibniz-Institut DSMZ-Deutsche Sammlung von Mikroorganismen und Zellkulturen, GmbH, on 16 Feb. 2022 under number DSM34172.

27. A lactose-positive, sucrose-negative, Streptococcus thermophilus strain selected from the group consisting of:
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM34172 [herein referred to as ST1 or DSM34172 strain] into which the sequence of the open-reading-frame (ORF) of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C} pglcU^{IN_T}, pglcU^{IN_C}, pglcU^{IS}), and optionally wherein the glcK gene encoding a glucokinase, contain the sequence as defined by SEQ ID NO:25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO: 71 or a ccpA variant sequence having at least about 90%, such as at least 95% identity with SEQ ID NO: 71 at least 95%;
a strain of a Streptococcus thermophilus, such as the strain deposited under the Budapest Treaty on Jan. 14, 2014 in the name of Danisco Deutschland GmbH at Leibniz-Institut DSMZ under number DSM34172 [herein referred to as ST1 or DSM34172 strain] comprising one or more of the following features:
1) into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}),
2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}),
4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO:167;
6) having a ccpA sequence of SEQ ID NO:71 or a ccpA variant sequence having at least about 90%, such as at least 95% identity with SEQ ID NO: 71 at least 95%;
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on Jan. 14, 2014 in the name of Danisco Deutschland GmbH at Leibniz-Institut DSMZ under number DSM28255 [herein referred to as ST4 or DSM28255 strain] into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO:213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO:25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO: 71 or a ccpA variant sequence having at least about 90%, such as at least 95% identity with SEQ ID NO: 71 at least 95%;
a strain of a Streptococcus thermophilus, such as the strain deposited under the Budapest Treaty on Jan. 14, 2014 in the name of Danisco Deutschland GmbH at Leibniz-Institut DSMZ under number DSM28255 [herein referred to as ST4 or DSM28255 strain] comprising one or more of the following features:
1) into which the sequence of the open-reading-frame (ORF) of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS}),
2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
6) having a ccpA sequence of SEQ ID NO: 71 or a ccpA variant sequence having at least about 90%, such as at least 95% identity with SEQ ID NO: 71 at least 95%;
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence upstream of the start codon of scrB gene is replaced by the corresponding sequence as set forth in SEQ ID NO:213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO:213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO:25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO:71 or a ccpA variant sequence having at least about 90%, such as at least 95% identity with SEQ ID NO: 71;
a strain of a Streptococcus thermophilus, such as the strain deposited under the Budapest Treaty on 18 Jan. 2022 in the name of DuPont Nutrition Biosciences ApS at Leibniz-Institut DSMZ under number DSM34132 [herein referred to as ST2 or DSM34132 strain] comprising one or more of the following features:
1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
2) the sequence upstream of the start codon of scrB gene is replaced by the corresponding sequence as set forth in SEQ ID NO: 213 or a variant sequence being at least about 90%, such as at least about 95% identical herewith (pscrB₈₂₉),
3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
5) having a mutation creating a frameshift of the open reading frame of the gene coding for the EIIC^{Man} protein leading to a stop codon at position 180 of SEQ ID NO:222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
6) having a ccpA sequence of SEQ ID NO:71 or a ccpA variant sequence having at least about 90%, such as at least 95% identity with SEQ ID NO: 71;
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO:212 or a variant sequence being at least about 90%, such as at least about 95% identical herewith resulting in a frameshift leading to the translation in a truncated EIIABC^{Sucrose} (scrA₈₂₉^{FS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉);
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of the scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}) and the sequence upstream of the start codon of scrB gene is replaced by the corresponding sequence as set forth in SEQ ID NO: 213 or a variant sequence being at least about 90%, such as at least about 95% identical herewith (pscrB₈₂₉) and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS});
a strain corresponding to the Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D-38124 Braunschweig-Germany] into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}), and the sequence upstream of the start codon of scrB gene is replaced by the corresponding sequence as set forth in SEQ ID NO: 213 or a variant sequence being at least about 90%, such as at least about 95% identical herewith (pscrB₈₂₉), and the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO:221 or a variant sequence being at least about 90%, such as at least about 95% identical herewith (pglcU^{SNP_C}, pglcU^{IN_T}, pglcU^{IN_C}, pglcU^{IS}), and optionally wherein the glcK gene encoding a glucokinase, contain the sequence as defined by SEQ ID NO:25 or a variant sequence being at least about 90%, such as at least about 95% identical herewith, wherein the amino acid at position 275 is a lysine (glcK₃₇₈), and optionally further comprising a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167; and optionally further comprising a ccpA sequence of SEQ ID NO: 71 or a ccpA variant sequence having at least about 90%, such as at least 95% identity with SEQ ID NO: 71;
a strain of a Streptococcus thermophilus strain deposited under accession number DSM33651 (herein referred to as ST3 or DSM33651) on Sep. 29, 2020, at the DSMZ [Deutsche Sammlung von Mikroorganismen und Zellkulturen GmbH, Inhoffenstrasse 7B, D38124 Braunschweig-Germany] comprising one or more of the following features:
1) into which the sequence of scrA is replaced by the corresponding sequence as set forth in SEQ ID NO: 212 or a variant sequence being at least about 90%, such as at least about 95% identical herewith resulting in a frameshift leading to the translation in a truncated EIIABC^{Sucrose} (scrA₈₂₉^{FS}),
2) the sequence upstream of the start codon of scrB gene is replaced by the corresponding sequence as set forth in SEQ ID NO:213 or a variant sequence being at least about 90%, such as at least about 95% identical herewith (pscrB₈₂₉),
3) the genetic locus that was the promoter region upstream of the glcU gene (pglcU) is replaced by the corresponding sequence as set forth in any one of SEQ ID NO: 217-220 or a variant sequence being at least about 90%, such as at least about 95% identical herewith, or which promotor comprises a sequence of SEQ ID NO: 221 or a variant sequence being at least about 90%, such as at least about 95% identical herewith (pglcU^{SNP_C}, pglcU^{IN_T}, pglcU^{IN_C}, pglcU^{IS}),
4) the glcK gene encoding a glucokinase, contain the sequence as defined by SEQ ID NO: 25 or a variant sequence being at least about 90%, such as at least about 95% identical herewith, wherein the amino acid at position 275 is a lysine (glcK₃₇₈),
5) having a mutation creating a frameshift of the open reading frame of the gene coding for the EIIC^{Man} protein leading to a stop codon at position 180 of SEQ ID NO: 222, such as by an insertion of one nucleotide C at any one of positions 438, 439, or 440 of SEQ ID NO: 223 in the manM gene (manM₁₂₉₉₇) and/or comprising a substitution of nucleotide G to T at position 916 of SEQ ID NO: 79 of the manL gene (manL₁₃₇₄₀) leading to a stop codon at position 306 of the protein of SEQ ID: 80 (IIAB^{Man}); and/or comprising a substitution of nucleotide G at position 625 of manM by a nucleotide T (manM₁₆₄₀₄), leading to a stop codon at position 209; and/or comprising the insertion of one nucleotide A at the stretch of 5 nucleotides A at positions 37-41 creating a frameshift in manN gene (resulting in manN₁₃₇₄₁ mutated allele) leading to a truncation of the IID^{Man} protein at position 28 of SEQ ID NO: 167;
6) having a ccpA sequence of SEQ ID NO:71 or a ccpA variant sequence having at least about 90%, such as at least 95% identity with SEQ ID NO: 71.

28. A polynucleotide comprising one or more mutation in one or more gene of the sucrose regulon, which one or more mutation provides for a significant reduction in the ability to uptake and consume any sucrose in a strain selected from:
a) a strain corresponding to the Streptococcus thermophilus strain deposited under the Budapest Treaty on Jan. 14, 2014 in the name of Danisco Deutschland GmbH at Leibniz-Institut DSMZ under number DSM28255, into which strain said polynucleotide is replacing the corresponding sequence in DSM28255;
b) a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132, into which strain said polynucleotide is replacing the corresponding sequence in DSM34132;
c) a strain corresponding to the Streptococcus thermophilus strain deposited under accession number DSM33651 on Sep. 29, 2020, at the DSMZ [Deutsche Sammlung von Mikroorganismen und Zellkulturen GmbH, Inhoffenstrasse 7B, D-38124 Braunschweig-Germany], into which strain said polynucleotide is replacing the corresponding sequence in DSM33651;
d) a strain corresponding to the Streptococcus thermophilus strain deposited under the Budapest Treaty on 16 Feb. 2022 in the name of DuPont Nutrition Biosciences ApS at Leibniz-Institut DSMZ under number DSM34172, into which strain said polynucleotide is replacing the corresponding sequence in DSM34172.

29. A polynucleotide comprising one or more mutation affecting the glucose porter, which one or more mutation restores or improves glucose consumption of a strain selected from:
a) a strain corresponding to the Streptococcus thermophilus strain deposited under the Budapest Treaty on Jan. 14, 2014 in the name of Danisco Deutschland GmbH at Leibniz-Institut DSMZ under number DSM28255, into which strain said polynucleotide is replacing the corresponding sequence in DSM28255;
b) a strain corresponding to the Streptococcus thermophilus strain deposited under the Budapest Treaty on 18 Jan. 2022 in the name of DuPont Nutrition Biosciences ApS at Leibniz-Institut DSMZ under number DSM34132, into which strain said polynucleotide is replacing the corresponding sequence in DSM34132,
c) a strain corresponding to the Streptococcus thermophilus strain deposited under accession number DSM33651 on Sep. 29, 2020, at the DSMZ [Deutsche Sammlung von Mikroorganismen und Zellkulturen GmbH, Inhoffenstrasse 7B, D-38124 Braunschweig-Germany], into which strain said polynucleotide is replacing the corresponding sequence in DSM33651;
d) a strain corresponding to the Streptococcus thermophilus strain deposited under the Budapest Treaty on 16 Feb. 2022 in the name of DuPont Nutrition Biosciences ApS at Leibniz-Institut DSMZ under number DSM34172, into which strain said polynucleotide is replacing the corresponding sequence in DSM34172.

30. A polynucleotide according to any one of claim 28 or 29, which one or more mutation is as identified in any one of claims 3-8.

31. A method for the selection of a lactose-positive, sucrose-negative, Streptococcus thermophilus strain, which method includes the steps of:
1) providing a strain of Streptococcus thermophilus including
a) one or more mutation in one or more gene of the sucrose regulon; which mutation provides for a significant reduction in the ability to grow on and/or transport sucrose by said strain; and which mutation at least comprises
i) one or more of the scrA gene, and/or the scrB gene and/or the scrR gene, and
ii) optionally a further mutation in the promoter region regulating the expression of the scrA, scrB and/or scrR genes, such as a mutation that protect the strain from reversing from the sucrose negative phenotype;
2) screen for a strain of Streptococcus thermophilus from step 1), which strain has a significant reduction in the ability to grow on and transport sucrose; and
3) optionally screen prior to, simultaneously with, or after any one of steps 1 and 2 for a Streptococcus thermophilus strain which has a restored or improved glucose consumption, optionally from one or more further mutation affecting the glucose porter, such as affecting glcU and/or its expression.

32. The method according to claim 31, which strain is as defined in any one of claims 1-21.
